(19) United States
(12) Patent Application Publication
(10) Pub. No.: US 2019/0048306 A1
(43) Pub. Date: Feb. 14, 2019

(54) Redox-related context adjustments to a bioprocess monitored by learning systems and methods based on redox indicators

(71) Applicant: BioElectron Technology Corporation, Mountain View, CA (US)

(72) Inventor: Stephen J. Brown, Woodside, CA (US)

(21) Appl. No.: 15/785,415

(22) Filed: Oct. 16, 2017

Related U.S. Application Data
(63) Continuation-in-part of application No. 15/675,364, filed on Aug. 11, 2017.

(51) Int. Cl. C12M 1/34; G05D 21/02; G06N 3/08

(52) U.S. Cl. CPC C12M 41/28; G05D 21/02; C12N 9/0004; C12M 41/26; G06N 3/08

(57) ABSTRACT

The present invention concerns methods and systems for learning or discovering redox-related context adjustments to a biological process or bioprocess experienced by one or more biological entities under local conditions. The bioprocess is postulated to have hidden states associated with redox reactions. Among other, the biological entities can be embodied by plants, animals, cells, cell cultures, cell lines and human subjects. The learning system uses a reference bioprocess model for the bioprocess and has a master learner configured to establish an observable basis of redox indicators for the bioprocess. The learning system also has a local learner in communication with the master learner. The local learner deploys a learning algorithm to learn an operator matrix that represents the redox-related context adjustment.

### CROSS-REFERENCE TO RELATED APPLICATIONS

The present application is a continuation-in-part of U.S. patent application Ser. No. 15/675,364 filed on Aug. 11, 2017 under the title “Distributed systems and methods for learning about a bioprocess from redox indicators and local conditions”. The present application is also related to provisional application 62/544,749 filed on Aug. 11, 2017 under the title “Monitoring and control of electron balance in bioreactor systems”.

### FIELD OF THE INVENTION

The present invention relates to apparatus and methods for applying distributed computer learning algorithms to bioprocesses at both the level of reduction-oxidation (redox) reactions that are not directly observable and thus assigned to hidden states, and at the level of local conditions under which the bioprocesses of interest occur in biological entities of interest. Relevant biological entities cover biological systems such as bioreactors, and also living entities such as live plants, animals, cells, cell cultures and human subjects.

### BACKGROUND OF THE INVENTION

By most definitions, all entities or systems undergoing a biological process or a bioprocess are considered to be alive. Living biological entities range from biological systems, e.g., biomasses in controlled bioreactors, to living organisms. The latter include animals and plants. Often, biological entities at this level are viewed in the context of their environments or local conditions that are either conducive to their existence or not.

Living entities on planet Earth can be broken down into bacteria, archaea and eukaryotes. Their sizes, from smallest to largest, span many orders of magnitude. The bioprocesses that these biological entities undergo are extremely varied and highly complex. The study of biological entities at this level belongs to the fields of biology, ecology, zoology and botany.

Despite the truly remarkable amount of differentiation among biological entities, they do share common structures and operating principles. One such operating principle is that all biological entities depend on harvesting external energy sources to stay alive. In terms of common structures, all biological entities, except perhaps viruses, are made up of a smallest basic living component: the cell. While being the smallest units of life, cells also coincide with the smallest living biological entities of interest: bacteria.

At the cell level, life is again found to exhibit myriads of complex structures and processes. The processes of interest happen here on much shorter time scales than at the higher level of multi-cellular biological entities. A new set of common operating principles and shared structures are found at the cell level.

In particular, processes occurring at the cell level are described by molecular biology and biochemistry. They can be understood in terms of biochemical structures and reactions. The most important biochemical reactions include construction, replication, feeding, repair, energy regulation, and carrying out of primary cell functions (dependent on cell type).

Below the cell level is the realm of processes and structures operating on still shorter time scales. It is the level of physical organic chemistry and, ultimately, quantum chemistry and quantum physics. The latter govern the actions of atoms and of small molecules by rules that transcend classical logic and assumptions. Even the ability to assign probabilities to measurements in this realm is conditional. It is preceded by operations on propensities that depend on context and are unobservable even in principle. (We are referring here to entities such as electron wave functions.) Still, common structures and processes are found even at this level.

Many approaches and techniques for understanding the structures and processes of physical organic chemistry have been proposed over the past fifty years. One prominent modeling approach attempting to explain the relationship between specific structures and activities is the Quantitative Structure-Activity Relationship (QSAR) model. QSAR was introduced by Corwin Hansch et al. in 1962. An excellent text describing this contribution and the consequent approaches developed from it is provided by Hugo Kubinyi, “QSAR: Hansch Analysis and Related Approaches”, Methods and Principles in Medicinal Chemistry, New York, 1993.

More recent 3D QSAR and Comparative Molecular Field Analysis (CoMFA) models have attempted to apply quantum-chemical tools to determine chemical reactivity at the level of physical organic chemistry. These models track the formation of hydrogen bonds, proton movement/hopping, electron exchanges and/or oxidation-reduction (redox) reactions as well as steric effects. The latter affect ligand binding preferences and are also related 3D alignment effects. Although the practice of 3D QSAR is inherently limited to local models at this level of study, it can be expected to make further progress. Specifically, the expansion of published databases such as ChEMBL and PubChem along with annotations and 3D alignment protocols, may continue to provide better validated physical organic chemistry models for both screening (e.g., drug or toxic substance screening) and machine learning applications in this field. An excellent summary of the present state of the art in this realm is afforded by Cherkasov, et al., “QSAR Modeling: Where have you been? Where are you going to?”, J. Med. Chem., Volume 57, No. 12, Jun. 26, 2014, pp. 4977-5010 and the numerous references cited therein.

Systems biology examines life as it builds on top of the low level of physical organic chemistry, which is in the purview of 3D QSAR and other Field Models addressed above. Systems biology is further informed by data collected in the various -omes, and in particular the genome and the proteome. In examining the Genome-Protein-Reaction (GPR) chain, systems biology brings to bear traditional tools of applied mathematics and linear algebra. It has attempted to deploy these tools to model biology in terms of metabolic networks, elements, reactions, fluxes as they act under certain constraints to achieve local equilibria or homeostasis. The differential equations of systems biology address processes that attempt to reach the level of entire cells and even entire multi-cellular biological entities. Systems biology has advanced the understanding of structure and biological function of simple single celled biological entities. For example, a curated genome-scale metabolic network reconstruction of Escherichia coli has been achieved in the recent past. A general review of the state of the art in systems biology is found in the textbook by Bernhard O. Palsson, “Systems Biology: Constraint-based Reconstruction and Analysis”, Dept. of Bioengineering, University of California San Diego, Cambridge University Press, 2015, and in the sources recited therein.

As is likely already clear from the above, division of life into various levels of study can only take us so far. Reconstruction from the genome information of the overall cell proteins and structure is not sufficient to tell us what regulatory processes are active at shorter time scales, e.g., in the physical chemistry layer. Thus, understanding the translation of the genetic code into proteins provides only a background against which the processes of physical chemistry unfold. Specifically, regulatory mechanisms involving the available enzymes that catalyze the millions of cell reactions occurring during each second have to be included in order to understand cell regulation. Still differently put, many of the crucial effects and regulatory mechanisms are found in the interstices between levels at which the life of the biological entity and its cells is being investigated. We also observe direct inter-level effects. Activity at the physical chemistry level, i.e., below the cell level, directly affects activity and structure at the cell level and at the level of the biological entity and its local conditions or environment.

These considerations bring back into focus the physical chemistry processes that involve the transfer of electrons and proton hopping. These processes are due to underlying field effects and molecular conformations (topology). They are generally known as reduction-oxidation reactions. Their effects occur at the cell level. Indeed, within any cell there are a number of specialized enzymes and affiliated compounds that are also involved in the regulation of these reactions. They include enzymes generally categorized as oxidoreductases, as well as their co-factors and other electron carrying molecules and/or complexes. These enzymes, co-factors and complexes participate in redox reactions to provide a critical level of balance and regulation for bioprocesses. For an introductory level review of these issues the reader is referred to standard texts, such as Bruce Alberts et al., “Molecular Biology of the Cell”, Garland Science, 5^{th} Edition, New York, 2008.

In their seminal article, Bucher, T. and Klingenberg M., “Pathways of hydrogen in the living organization”, Angewandte Chemie (Applied Chemistry), 70, pp. 225-570, 1958 examined the pathways of hydrogen in a living organization of a biological system or biological entity (bio-entity). This study addressed the interactions within the network of redox reactions extending over essential functions of living cells. The crucial nature of redox systems and redox reactions in bioprocesses occurring in biological systems and entities was thus firmly established. A redox code for classifying redox reactions was developed. The redox code consists of four principles by which biological systems and entities are organized.

The first redox principle is the use of the reversible electron accepting and donating properties in NAD and NADP to provide organization of metabolism (at or near equilibrium). The second redox principle is the use of redox electron transfers to adjust protein structure through kinetically controlled redox switches (a.k.a. S-switches or Sulphur switches) in the proteome to control tertiary structure, macromolecular interactions and trafficking, activity and function. The third redox principle is redox sensing as used in activation/deactivation cycles of redox metabolism, especially involving H₂O₂, support of spatiotemporal sequencing in differentiation and life cycles of cells and biological entities, e.g., organisms. The fourth principle is that redox networks form an adaptive system to respond to local conditions including the external environment. This adaptive system extends from micro-compartments through subcellular systems to the level of the cell and still further to tissue organization. A detailed explanation of these four redox principles is found in Jones, Dean P. et al., “The Redox Code”, Review Article appearing in Antioxidants and Redox Signaling, Vol. 0, No. 0, 2015, pp. 1-14. Further background provided by the same main author on select redox couples can be found in Jones, Dean P. et al., “Cysteine/cysteine couple is a newly recognized node in the circuitry for biologic redox signaling and control”, The FASEB Journal, Vol. 18, August, 2004, pp. 1246-1248.

Certain redox reactions and the electron balances they establish have been proposed to monitor cell status (e.g., oxidative stress) in some contexts. For example, U.S. Pat. No. 9,273,343 to Cali et al. suggests the use of compounds and methods for assaying the redox state of metabolically active cells and for measuring NAD(P)NAD(P)H balance. Tracking of certain redox reactions in conjunction with genome-scale metabolic network reconstruction has also been considered in U.S. Pat. No. 8,311,790 to Senger et al. This teaching addresses the identification of incomplete metabolic pathways to allow for the completion of genome-scale metabolic network for C. acetobutylicum. The program could thus provide a potential model of a genome-scale stoichiometric matrix that could attempt to model cell growth in silico.

The use of redox reactions for detecting certain analytes has also been investigated beyond the normal cell environment, e.g., in vitro. For example, U.S. Pat. No. 7,807,402 to Horn et al. proposes a method and reagent for detecting the presence and/or the amount of a certain analyte by a redox reaction and a fluorimetric determination. The redox reaction would be monitored here by a certain redox indicator. The oxidizing or reducing system would act directly on the redox indicator or via a mediator. The presence of the analyte would result in a reduction or oxidation of the redox indicator, which would allow for a qualitative or quantitative determination. U.S. Pat. No. 9,605,295 to Yau suggests an ultrasensitive and selective system and method for detecting certain reactants of the chemical/biochemical reaction catalyzed by an oxidoreductase. The action of the electrical field is suggested to facilitate the interfacial electron transfer between oxidoreductase and the working electrode of his electrochemical system by the quantum mechanical tunneling effect. Additional teachings of Yau involving bio-reactive systems and their voltage-controlled metabolism are found in U.S. Pat. Appl. No. 2016/0333301.

U.S. Pat. Appl. No. 2016/0166830 to Avent et al. illustrates the difficulties in devising systems, devices and methods to selectively provide antioxidant or pro-oxidant effects to control free radical damage in an organism. The therapeutic electron and ion transfer via half-cell involves providing electrodes, which may include syringe needles, to establish conductive paths to or from the organism, e.g., a human patient.

In principle, a needle-type testing apparatus could be miniaturized and improved by leveraging MEMS technologies for specific analytes. Examples of such apparatus and methods proposed to measure certain chemical species in biological samples, including certain specific reduction-oxidation potentials are found in the literature. The reader is referred to Hyoung-Lee, W. et al., “Needle-type environmental microsensors: design, construction and uses of microelectrodes and multi-analyte MEMS sensor arrays”, Measurement Science and Technology, Vol. 22, March 2011 (22 pgs.) and to Lee, Jin-Hwan et al., “MEMS Needle-type Sensor Array for in Situ Measurements of Dissolved Oxygen and Redox Potential”, Environmental Science and Technology, Vol. 42, No. 22, 2007, pp. 7857-7863.

Clearly, access to observing hidden states even with highly specific targets within a functioning cell or organism remains a challenge. Thus, despite the advanced state of the art with respect to very specific redox reactions with known functions, the study of biological entities and systems in light of the redox reactions they undergo lacks in proper contextualization. Differently put, the local conditions under which the biological entities experience the bioprocesses need to be reflected in the systems that learn and produce the models. Given the multitude of processes and structures at the many levels or scales on which life transpires, it is important to use models of redox reactions and measurements obtained via appropriate redox indicators in a more complete and context-sensitive manner.

What is lacking are learning systems and methods that measure a broader set of chemicals and other redox data and identify patterns of potential redox indicators from alternative compartments and/or from otherwise imprecise sensors. It would be desirable for such learning systems and methods to learn new patterns from field or local measurements in learned local contexts, rather than only in the highly controlled lab environment.

### OBJECTS AND ADVANTAGES

In view of the shortcomings of the prior art, provided herein are learning systems and methods that deploy distributed learning algorithms in a manner that permits improved learning from redox reactions under local conditions in which the biological entity of interest is embedded.

In addition, the systems and methods described herein may reduce reliance on expensive laboratory testing equipment in lab settings and to promote less expensive field or local measurement systems. Use of less expensive equipment and sensors can still be effective in estimating redox data under generally less controlled local conditions where one or more biological entities are undergoing the bioprocess of interest. This can be addressed through the application of distributed learning.

Also provided are distributed learning algorithms that adjust for inter-level relationships between processes and structures in light of redox reactions.

Distributed learning algorithms that learn about redox indicators and appropriate observable bases of such redox indicators in light of local conditions are also provided.

These and other objects and advantages of the invention will become apparent upon reading the detailed specification and reviewing the accompanying drawing figures.

### SUMMARY OF THE INVENTION

The present invention relates to computer implemented learning methods and systems that can learn about redox-related context adjustments to a biological process or bioprocess. The bioprocess is experienced by one or more local biological entities. Each of the local biological entities experiences the bioprocess under their own local conditions and generates measured redox data for the bioprocess.

Given that the redox status is not a directly observable parameter of any typical biological system under local conditions it will be considered as indirect, inferred or otherwise derived knowledge. Correspondingly, the bioprocess is postulated to have hidden states that are not directly observable by measuring equipment or sensors deployed under local conditions. The hidden states may, and in typical embodiments of the present invention will, include unknown states beyond those of just the redox status of the bioprocess that the biological entity is experiencing.

The learning system has a reference bioprocess model configured to yield model redox data for the bioprocess. Reference bioprocess model can be obtained from curated model reference data collected from previous tests of the bioprocess. Such model redox data may be further labeled, classified or annotated by experts. Alternatively, or in addition, the reference bioprocess model can be obtained from a reference biological entity that undergoes the process under model conditions. Such reference biological entity may be used to corroborate an already existing bioprocess reference model or even as the only source of the model.

Model redox data should be such that a master learner configured to receive it is able to establish from it an observable basis of redox indicators. An observable basis excludes any hidden states or otherwise hidden or inaccessible data. Thus, any vector spaces established using the observable basis of redox indicators are real-valued and measurable. Any candidate redox indicators in such vector spaces can be assigned real values and measured. Further, master learner is also configured to establish from the model redox data a model feature vector that expresses some or all of the model redox data in the observable basis.

The learning system has a local learner typically capable of being implemented in a hardware unit with lower measuring and processing capabilities, lower-power, or lower-bandwidth requirements in comparison to the measuring and processing capabilities of the reference bioprocess model and its references. The local learner is configured to receive at least a portion of model redox data from the reference bioprocess model. This portion may contain only model redox data relevant to local conditions or otherwise limited model redox data. The model redox data may also contain an initial reference learning model and any initial weights or starting points for the local learner.

The local learner is further configured to express the measured redox data it receives from any of the local biological entities undergoing the bioprocess by a measured feature vector. The measured feature vector is expressed in the observable basis established by the master learner.

The learning system deploys a learning algorithm that is preferably distributed. The learning algorithm learns an operator matrix that will transform between the model feature vector and the measured feature vector. In other words, the learning algorithm is applied to estimating an operator matrix that, when applied to model feature vector will yield the measured feature vector. The redox-related context adjustment is then taken as being at least partly represented by the operator matrix.

The local biological entity undergoing or experiencing the bioprocess can cover many types of entities. These range from cells, cell lines, cell cultures to biomasses. Any of these may experience the bioprocess in a bioreactor. Local biological entities may also be embodied by living entities, such as plants, organisms, animals, and human subjects. These will typically experience the bioprocess under their standard local conditions, e.g., in their natural habitats.

The learning system may be further equipped with a context classifier for associating the operator matrices discovered by the learning algorithm with local conditions. In other words, the context classifier associates a specific operator matrix that transforms from model feature vector obtained under lab or model conditions to the specific local conditions in which the given local biological entity is embedded. Such context classifiers may further associate any given operator matrix with a diagnosis of the corresponding local biological entity. For convenience, the context classifier may further associate operator matrices with context labels for easier accessing, sharing and searching.

In some embodiments, a local feedback mechanism is provided between the local learner and the local biological entity. The local feedback mechanism can apply the redox related context adjustment discovered by the learning algorithm to the local biological entity. In such embodiments, any actuators or other devices may be included in the local feedback mechanism. The actuators or devices may be configured to operate on at least one control parameter that affects the local conditions and hence the conditions under which the local biological entity experiences the bioprocess. The control parameter or parameters may relate directly to the redox state. In general, the control parameter can be a redox active compound or an electron balance influencer, or still other input that can act upon the bioprocess transpiring in the local biological entity under local conditions.

Well established and commonly accepted redox indicators may also be referred to as electron balance indicators. Particularly useful and established electron balance indicators include indicators consisting of an oxidoreductase, an oxidoreductase co-factor, an electron balance influencer compound, an electron balance influencer composition, a redox-active compound, a pK value, a pH value, a threshold value, a context measure and a soft indicator.

Furthermore, it is known that useful redox indicators or electron balance indicators should be measured or acted upon on short time scales in comparison to GPR times. Hence in advantageous embodiments the at least one electron balance indicator is measured or acted upon with a frequency of at least once every hour, at least once every 30 minutes, at least once every 10 minutes, at least once every 5 minutes, at least once every minute, at least once every 30 seconds, at least once every 10 seconds, at least once every 5 seconds, at least once every second, at least twice every second, at least 5 times every second, at least 10 times every second, at least 20 times every second, at least 50 times every second, at least 100 times every second, or more.

In certain cases, the local feedback mechanism will be a secondary feedback loop established between the local learner and the local biological entity. The local feedback mechanism should be appropriately provisioned to perform any local conditions adjustment represented by the operator matrix.

The learning system can employ many general methods that extend beyond the method used by the learning algorithm. In other words, the learning algorithm that engages in learning the operator matrices and their associations with local conditions adjustments need not be implemented within any one particular learning paradigm. In fact, the learning system can employ one or more learning methods. Some particularly useful methods in the embodiments of the present invention include Artificial Intelligence (AI) methods, Hidden Markov methods and Deep Learning (multi-layered neural network) methods. Any of these methods can be implemented in the recursive feedback structure presented by the learning system of the invention.

In general, and independent of the selection of control parameters, and observable redox indicators the redox data should contain at least one known and reliable redox indicator and at least one well known electron balance influencer.

The computer implemented learning methods learn about redox-related context adjustments to the biological process or bioprocess that has hidden states. The method uses one or more local biological entities placed under their own local conditions for experiencing the bioprocess and for generating measured redox data for the bioprocess.

The learning method uses a reference bioprocess model for obtaining model redox data for the bioprocess. Model redox data is transmitted to a master learner configured to receive it and to establish from it an observable basis of redox indicators. An observable basis excludes any hidden states or otherwise hidden or inaccessible data. Thus, any vector spaces established using the observable basis of redox indicators are real-valued and measurable. Any candidate redox indicators in such vector spaces can be assigned real values and measured. Further, master learner is also configured to establish from the model redox data transmitted to it a model feature vector that expresses some or all of the model redox data in the observable basis.

The learning method uses a local learner typically capable of being implemented in a hardware unit with lower measuring and processing capabilities, lower-power, or lower-bandwidth requirements in comparison to the measuring and processing capabilities of the reference bioprocess model and its references. The local learner is configured to receive at least a portion of model redox data from the reference bioprocess model. This portion may contain only model redox data relevant to local conditions or otherwise limited model redox data. The model redox data may also contain an initial reference learning model and any initial weights or starting points for the local learner.

The local learner is further configured to express the measured redox data it receives from any of the local biological entities undergoing the bioprocess by a measured feature vector. The measured feature vector is expressed in the observable basis established by the master learner.

The learning method deploys a learning algorithm that is preferably distributed. The learning algorithm learns an operator matrix that will transform between the model feature vector and the measured feature vector. The redox-related context adjustment is then taken as being at least partly represented by the operator matrix. The method of invention may include steps of associating the operator matrices discovered or learned by the learning algorithm with context classifiers, diagnoses, context labels and the like.

The method deploys a learning algorithm that is preferably distributed. The learning algorithm learns the redox-related context adjustment to the local bioprocess based on the operator matrix established by the learning algorithm. The learning is preferably performed on time-scales consistent with changes in redox-related indicators, as indicated above. Suitable learning methods include at least an Artificial Intelligence method, a hidden Markov method, a Deep Learning method.

The present invention, including the preferred embodiment, will now be described in detail in the below detailed description with reference to the attached drawing figures.

### BRIEF DESCRIPTION OF THE DRAWING FIGURES

FIG. 1A is a high-level diagram of the main parts of a learning system in accordance with the invention in which the biological entity of interest is a bioreactor

FIG. 1B is a high-level diagram of the main parts of a learning system in accordance with the invention in which several local biological entities of interest are live subjects

FIG. 2A is a diagram illustrating an exemplary set of measured redox data

FIG. 2B is a diagram illustrating an exemplary subset of redox data and an exemplary optimal composition of measured redox data

FIG. 2C is a diagram showing the transmission of measured redox data from a subject under local conditions and model redox data from the reference bioprocess model to the master learner

FIG. 2D is a diagram showing the representation of hidden states in the model used by the learning algorithm

FIG. 2E is a diagram showing the details of transitions between hidden states, measurement probabilities and assignment of confidence levels and weightings

FIG. 3 is a diagram illustrating an embodiment using a joint feature vector and deploying a neural net in the learning model of the distributed learning algorithm

FIG. 4A is a diagram illustrating local bioprocess occurring under local conditions with adjustments to local control parameters by a primary feedback mechanism

FIG. 4B is a diagram illustrating local bioprocess occurring under local conditions with adjustments to local control parameters by a local feedback mechanism

FIG. 5 is a diagram illustrating a reference bioprocess performed in a reference bioreactor with adjustments to reference control parameters

FIG. 6 is a diagram illustrating a preliminary learning model with abstract representation of the hidden states

FIG. 7 is a diagram illustrating a learning system configured to learn a redox-related context adjustment to a bioprocess experienced under local conditions

FIG. 8A is a diagram illustrating the application of a context matrix to a joint feature vector to obtain a model feature vector in canonical form

FIG. 8B is a diagram illustrating the application of an operator matrix to transform between model feature vector and measured feature vector

FIG. 8C is a diagram showing the operation of a local feedback mechanism that apply redox-related context adjustments encoded in operator matrices

FIG. 8D is a diagram illustrating a portion of the learning system of FIG. 7 adapted to use a simple context matrix

FIG. 9 is a flow diagram illustrating an exemplary application of the learning system of FIG. 7

### DETAILED DESCRIPTION

The drawing figures and the following description relate to preferred embodiments of the present invention by way of illustration only. It should be noted that from the following discussion many alternative embodiments of the methods and systems disclosed herein will be readily recognized as viable options. These may be employed without straying from the principles of the claimed invention. Likewise, the figures depict embodiments of the present invention for purposes of illustration only.

### General Configuration of Learning System

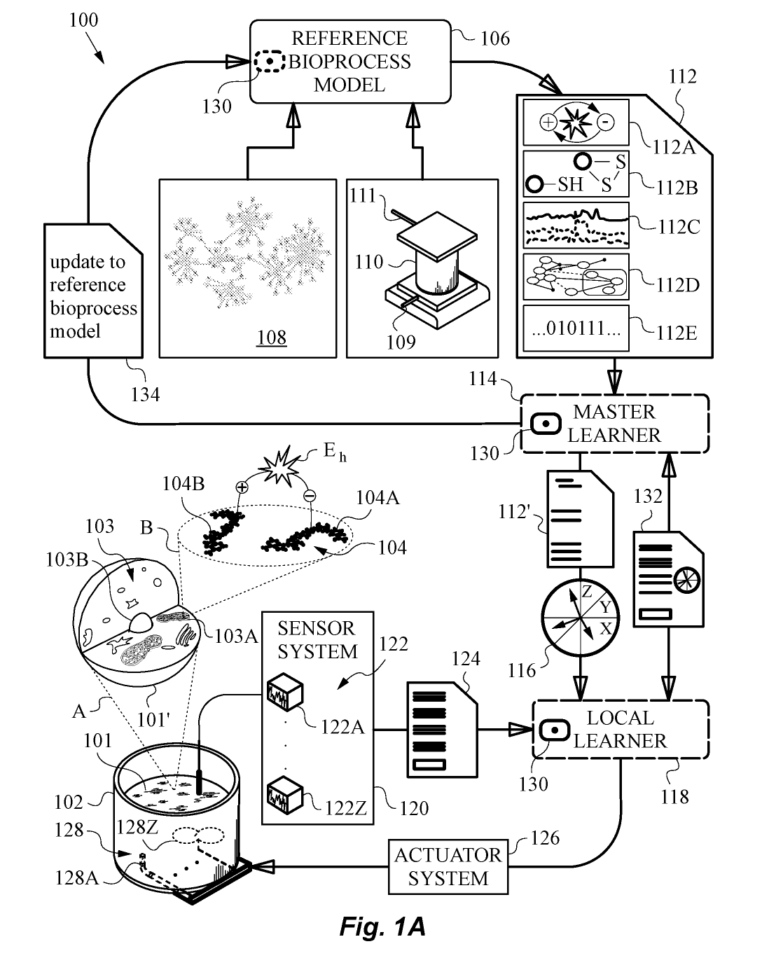

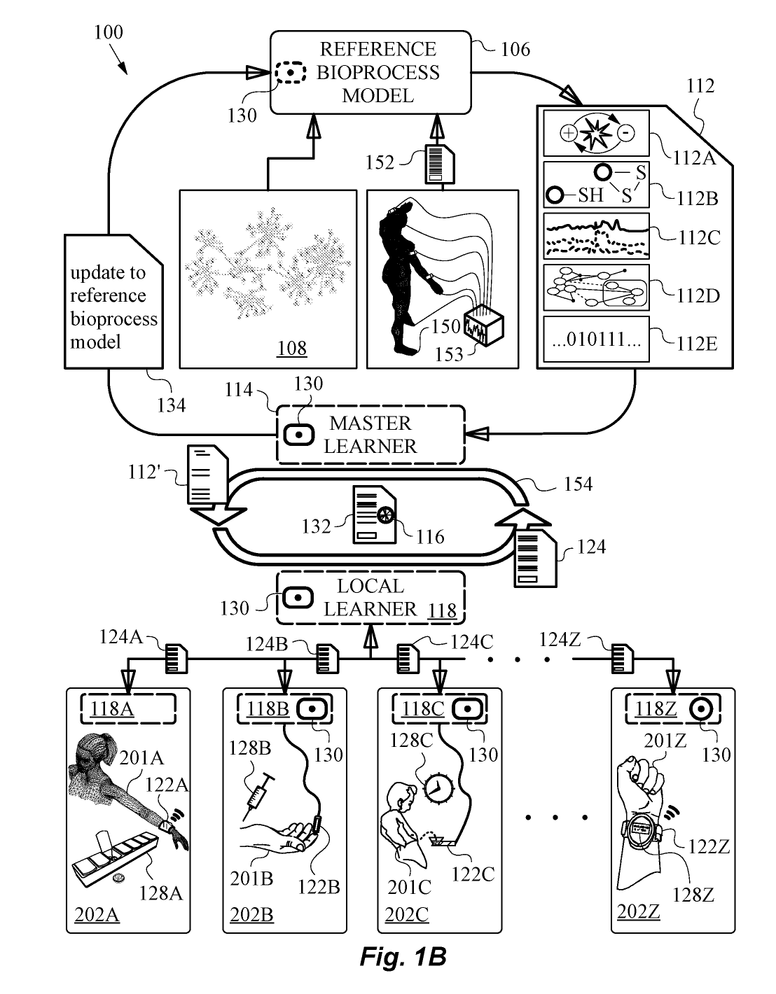

Computer implemented learning methods and systems described herein will be best appreciated by initially reviewing the high-level diagram of FIG. 1A. This diagram shows the main parts and interconnections of a learning system 100 configured to learn about a redox status of a biological process or bioprocess. The bioprocess is being experienced by a local biological entity 101. In this example, local biological entity 101 is a biomass, a cell culture, one or more organisms, a biomaterial or a biologically active substance or substances undergoing the bioprocess of interest in a bioreactor 102.

Bioreactor 102 should be understood to include dedicated reactors as well as incidental mechanisms, and even live systems. A person skilled in the art will thus appreciate that many types of in vitro and in vivo bioprocesses fall within this category. In the present exemplary embodiment, biological entity 101 is undergoing the bioprocess of interest within local bioreactor 102. Thus, local conditions experienced by biological entity 101 are those existing or sustained inside bioreactor 102.

Bioprocesses of interest in the present invention involve those that include reduction-oxidation reactions. To appreciate these types of reactions, FIG. 1A presents a first highly magnified section A of local biological entity 101 that is sufficiently enlarged to show one of its cells 101′. First section A helps to visualize the scale difference between the macroscopic level of entity 101 found inside bioreactor 102 and the microscopic level of cell 101′. At the cell level, exemplary cell 101′ is seen in a partial cut-away view to expose some common cell-level structures 103. Cell structures 103 include organelles familiar to those skilled in the art, such as mitochondria 103A and nucleus 103B surrounded by the cytosol (not expressly labeled).

FIG. 1A includes a second highly magnified section B that expands even further from section A. Section B magnifies a tiny volume within mitochondria 103A belonging to cell 101′. Second section B brings out a redox pair or redox couple 104. At the level of magnification afforded by section B, we see redox couple 104 at the physical chemistry level or layer. The molecular structures of redox pair 104 are visible at this level. Actual redox reactions occur at this level or scale. They typically involve the transfer of hydrogens or electrons and are thus often referred to as electron balance reactions.

FIG. 1A illustrates individual molecules 104A and 104B belonging to redox couple 104. For exemplary purposes only, molecule 104A is the NAD+ (Nicotinamide adenine dinucleotide) coenzyme molecule being reduced as indicated by the minus charge. Molecule 104B is the partner NADH molecule being oxidized, as indicated by the plus charge. The energy involved in the process is indicated by the voltage or potential difference ΔV, which is simply equal to the redox potential Eₕ. The exact numeric value of redox potential Eₕ will depend on departure of thermodynamic conditions from standard conditions, as described by the well-known Nernst equation Eₕ=Eₒ+RT/nF·ln([A]/[B]). Here Eₒ is the standard potential for the redox couple, R is the ideal gas constant, T is the absolute temperature in degrees Kelvin, n is the number of electrons transferred in the redox reaction and F is Faraday's constant. We use the natural logarithm of the ratio of concentrations (indicated by square brackets) of the oxidized and reduced members of the redox couple A, B (e.g., NAD+ and NADH, glutathione couple GSH/GSSH or cysteine and cystine couple Cys/CySS). Those skilled in the art will also be aware of still other parameters and factors that need to be considered in assessing the redox potential of any particular redox couple (e.g., whether it is in cell, in vivo, in vitro, in plasma, etc.).

The reader is cautioned not to rely unduly on the visual representation of the redox reaction shown in FIG. 1A. The quantum mechanical process of charge transfer involves the overlap of wave functions or propensities that cannot even in principle be fully represented in 3-dimensional space (R³). It is the overlaps of these unobservable propensities in a higher-dimensional and complex-valued space (Hilbert space) that “cause” the charge transfer. Specifically, they permit new topologies (i.e., field effects not supported in R³) that in turn dictate the probabilities for any particular type of electron or ion transfer process(es). Only the final charge transfer becomes a measurable, an observable or otherwise “classical quantity” associated with molecules, e.g., redox partners 104A and 104B. Due to these fundamental limitations and the complex environment inside cell 101′, the redox status of any particular reaction partners may not be directly observable.

In contrast, the redox status of a comparatively large number (e.g., hundreds or thousands) of redox couples or of more complex systems becomes measurable, especially under lab conditions. On large scales, electron balance induces changes in well-known parameters, e.g., the pH value (which is a common measure of H⁺ ion concentration in moles per liter of solution expressed on a logarithmic scale). Persons skilled in the art will be very familiar with measurements of redox status using such parameters. These parameters are commonly referred to as electron balance indicators or redox indicators. Depending on conditions and available equipment, the most useful group of redox indicators can include certain oxidoreductases, oxidoreductase co-factors, electron balance influencer compounds, electron balance influencer compositions, redox-active compounds, pK values, pH values, threshold values, context measures and soft or derived indicators (usually derived with reference to a mathematical model).

Unfortunately, under local conditions within bioreactor 102 where bioprocess transpires in biological entity 101, lab equipment is generally not available. Correspondingly, the bioprocess and specifically its model is postulated to have hidden states that are not directly observable by measuring equipment or sensors deployed under local conditions. The hidden states may, and in many cases indeed will, include unknown states beyond those of just the redox status of the bioprocess that local biological entity 101 is experiencing.

The high-level diagram in FIG. 1A lays out a generalized representation of learning system 100. It also shows a general apparatus used by learning system 100 to learn, measure and control or adjust the redox status of the bioprocess that local biological entity 101 is undergoing. The bioprocess from which learning system 100 learns or on which it trains is a reference bioprocess model 106. Reference bioprocess model 106 typically includes an initial or reference learning model. Reference bioprocess model 106 is derived from curated reference model redox data 108 collected from previous runs and tests of the bioprocess. Such curated model redox data 108 may further be labeled, classified or annotated by experts, as is common in this field and known to those skilled in the art.

In some cases, as seen in the present exemplary embodiment, reference bioprocess model 106 is further corroborated. Here, the corroboration is obtained from redox data collected from a reference bioreactor 110 that is undergoing the bioprocess of interest. Reference bioreactor 110 is preferably located in a controlled facility.

It should be noted that in cases where curated model redox data 108 is unavailable, model 106 can be derived from just the redox data collected from reference bioreactor 110. In other words, reference bioprocess model 106 can be derived empirically from a reference run of the same bioprocess as the one being performed or experienced by biological entity 101 in local bioreactor 102. It is desirable to combine empirical data from reference bioreactor 110 with curated model redox data 108 to obtain as complete a reference bioprocess model 106 as is practicable under the specific conditions that are likely to correspond to local conditions.

An input 109 to reference bioreactor 110 is provided for adjusting or altering the bioprocess occurring inside it. Input 109 is to be understood generally as any mechanism, actuator, inlet or other type of mechanical or non-mechanical apparatus capable of acting on the bioprocess. Likewise, an output 111 is provided for drawing outputs or samples from the bioprocess unfolding inside reference bioreactor 110. Actuator systems or mechanisms interfacing with input 109 and sensing or measuring apparatus interfacing with output 111 will be discussed in conjunction with specific embodiments and are therefore not shown in the present high-level diagram of FIG. 1A.

Reference bioprocess model 106 typically runs on a dedicated computer, computer system or even a computer cluster that is collocated or geographically distributed (not shown). Specific computer infrastructure and interfaces will depend on whether reference bioprocess model 106 relies on just curated model redox data 108, or empirical data obtained from reference bioreactor 110, or both. A person skilled in the art will appreciate, that many types of resources and architectures can support the running of reference bioprocess model 106. Herein, when referring to any inputs or outputs of reference bioprocess model 106 we mean the inputs and outputs of the computer or computer system(s) that actually implement(s) or run(s) reference bioprocess 106.

Reference bioprocess model 106 is designed to provide, output or yield model redox data 112 along with a preliminary, initial or reference learning model. Given that redox status is not a directly observable parameter of the bioprocess, knowledge about it will be considered herein as indirect, inferred or otherwise derived knowledge. Correspondingly, the bioprocess is postulated to have hidden states. These will typically be reflected in the reference learning model. The hidden states are ones that include redox status micro-states as well as states that are due to redox reactions, are affected by or related to redox reactions, or are otherwise dependent on electron transfer and/or balance during the bioprocess. As already indicated above, the extremely rapid and typically inaccessible nature of individual redox reactions renders them as prime candidates for hidden state representation. The hidden states may, and in typical embodiments of the present invention do include unknowable states. The unknowable states can extend beyond just those that are related to redox status of the bioprocess of interest. Model redox data 112, also frequently referred to herein just as model data or redox data 112, can be subdivided into several broad categories based on the redox code. The redox code includes the four principles by which biological systems are organized. The first category contains bio-energetics redox data 112A. These are data pertaining to catabolism and anabolism typically organized through high-flux NAD and NADP systems. The second category contains macromolecular structure and activities that are linked to bio-energetic systems through kinetically controlled sulfur switches. This category will be referred to herein as switching redox data 112B. The third category contains signaling redox data 112C. This category relates to activation and deactivation cycles, e.g., of H₂O₂ production (usually linked to NAD and NADP systems to support redox signaling and spatiotemporal sequencing for differentiation and multicellular development). The fourth category contains network redox data 112D. This type of data relates to redox networks, from micro-compartments to subcellular and cellular organization and includes adaptive responses to the environment.

In addition to the four redox code categories, model redox data 112 also contains a fifth category. This fifth category includes contingent redox data 112E. Contingent redox data 112E includes candidates (e.g., candidate redox indicators that are speculative) for any of the first four categories, as well as contextual information having to do with local conditions or environment in which reference bioprocess transpires. Contingent data 112E can also include other types of information that may be relevant directly or indirectly to oxidation-reduction activity or charge balance. It is possible for contingent redox data 112E to encompass contextual information that can only be inferred from factors not specifically related in any known way to charge balance. Contingent redox data 112E can also include common annotations, labels and other information that curators or experts typically add to ensure proper understanding of the data. Reference bioprocess model 106 is set up to yield each type of redox data 112A-E. In other words, all or some of bio-energetics redox data 112A, switching redox data 112B, signaling redox data 112C, network redox data 112D and contingent redox data 112E are output by reference bioprocess model 106 for the given local conditions. What is important is that bioprocess model 106 be configured to yield model redox data 112 about the bioprocess that will be useful. This is required despite the fact that the redox status is not a directly observable aspect of either reference bioprocess model 106 based on the bioprocess taking place in reference bioreactor 110, or of the bioprocess occurring in biological entity 101 in local bioreactor 102. In other words, a judicious choice of what to include in model redox data 112 is required to operate learning system 100. This choice involves selecting the appropriate candidates in all or some of the five categories 112A-E that constitute model redox data 112, as discussed in more detail below.

Reference bioprocess model 106, or more specifically the computer or computer system on which it is running, is in communication with a master learner 114. Master learner 114 can operate on the same computer or computer system(s) or another computer or computer system(s). In any event, master learner 114 is configured to receive model redox data 112 from reference bioprocess model 106. In the event biological entity 101 undergoing the bioprocess in local bioreactor 102 requires frequent or even continuous monitoring, the delay in the communication of model redox data 112 to master learner 114 should be kept as short as practicable. In such cases, geographic collocation of the computers or even operating both reference bioprocess model 106 and master learner 114 on the same computer is preferred. A person skilled in the art will be able to make the appropriate decision about the distribution and assignment of the correspondent computational tasks.

In accordance with the invention, master learner 114 is capable of establishing from model redox data 112 an observable basis of redox indicators 116. More specifically, master learner 114 is capable of establishing from knowledge of one or more or a combination of features from one or more of the five categories of redox data 112A-E observable basis of redox indicators 116. In the context of the systems and methods described herein, observable basis 116 has a mathematical meaning. It is a basis for a vector space that is postulated to be real-valued, or real. That is because observable basis 116 established by master learner 114 excludes any hidden states or otherwise hidden or inaccessible information.

Although FIG. 1A illustrates observable basis 116 to include only three vectors in a three-dimensional vector space established by generally known orthonormal basis vectors X, Y and Z it is understood that the vector space is typically of a much higher dimension than three. Any vector space or spaces established using observable basis of redox indicators 116, which we will frequently simply refer to as observable basis 116, are necessarily real-valued and measurable. A consequence of this choice is that any candidate for observable basis 116 in such vector spaces can be measured and assigned real values.

In establishing observable basis 116 of redox indicators master learner 114 should take into account the control and measuring affordances available to entire learning system 100, and especially to local bioreactor 102. These include any constraints of the local measurement system such as availability or accuracy of measurements under local conditions. These will be typically parts of the feedback mechanisms including, in particular, the local and the reference feedback mechanisms, as discussed in more detail below.

Learning system 100 is also equipped with a local learner 118. In most embodiments, local learner 118 is implemented in a low-power and low-bandwidth unit. Such unit is not expressly shown in FIG. 1A. Local learner 118 may possess the processing capabilities of a personal computer, a smart phone or a smaller embedded system. Furthermore, it may be implemented in a mobile unit with limited on-board resources and data access. It may be implemented on a local unit that accesses remote or cloud computing capabilities as needed for specific computations or requirements. Normally, however, local processing may be constrained by local processing power, latency, bandwidth or time requirements. The precise local conditions or field conditions under which local learner 118 is deployed may vary. Several examples will be discussed in conjunction with specific embodiments that will be discussed below. In any event, local learner 118 will typically use all the data that it does receive in an efficient manner.

Local learner 118, or more specifically the unit on which local learner 118 is implemented, is connected to a test or sensor system 120. In turn, sensor system 120 interfaces with local bioreactor 102. Sensor system 120 deploys one or more individual sensors or measurement devices 122 to collect measured redox data 124 from the bioprocess running in local bioreactor 102. In the exemplary embodiment of FIG. 1A a number of measurement devices 122 are deployed to collect measured redox data 124 from local bioreactor 102. Only measurement devices 122A and 122Z are expressly called out for reasons of clarity. It is noted that in some embodiments sensor system 120 may only utilize a single sensor or measurement device, e.g., just device 122A. It is understood that sensor system 120 may be connected to measurement devices 122 indirectly or by means of a data output or file export and data input or file import that includes a manual or hybrid process.

Biological system 101 experiences the bioprocess within local bioreactor 102 for which reference bioprocess model 106 has been prepared, configured or calibrated under lab conditions. Rather than starting without guidance, local learner 118 can be initialized with reference learning model obtained from reference bioprocess model 106 passed on by master leaner 114. Thus, local learner 118 can immediately look for structure in the redox data being collected from local bioreactor 102.

As in the case of local learner 118, local bioreactor 102 is usually a reactor with a significantly down-scaled measurement or sensor system 120. More precisely, it is considered down-scaled in comparison with reference bioreactor 110 that learning system 100 may use to obtain a large number of measurements of various types of redox data. Local bioreactor 102 can be implemented under known or previously tested local conditions. These known local conditions may correspond to just a small subset of model conditions under which reference bioreactor 110 has been or is being operated. The known local conditions may also correspond to just a small subset of model conditions under which curated model redox data 108 has been collected and on which reference bioprocess model 106 and its reference learning model are built.

It is also possible that local bioreactor 104 is implemented under unknown local conditions. Conditions are unknown when neither curated model redox data 108 nor reference bioreactor 110 have undergone the bioprocess of interest under model conditions that replicate local conditions or allow to reliably extrapolate to local conditions. Thus, reference bioprocess model 106 with its reference learning model and model redox data 112 may not properly reflect how bioprocess in local bioreactor 102 may progress under local condition. Under these circumstances, local bioreactor 102 and measured redox data it collects from biological system 101 can be used by learning system 100 to refine reference bioprocess model 106. This mode of operation and on-the-fly learning will be discussed in more detail below.

Sensor system 120 is configured to collect a set of measured redox data 124 from biological entity 101 undergoing the bioprocess of interest inside local bioreactor 102. Measured redox data 124 can contain any of the four redox code categories 112A-D as well as the fifth category of contingent redox data 112E that includes candidates and accounts for local conditions and any other contextual factors. In the embodiment shown in FIG. 1A, measured redox data 124 contains all five categories of redox data.

Measured redox data 124 can include information that is not directly measurable, also known herein as “soft data”. Such “soft data” is inferred on a model applied to a collection of surrogate measures that are weighted to estimate or infer a measure of interest. For more information about soft sensors and soft data the reader is referred to Paulsson D., et al., “A Sensor for Bioprocess Control Based on Sequential Filtering of Metabolic Heat Signals”, Vol. 14, Sensors, 26 Sep. 2014, pp. 17864-17882.

Due to local limitations, sensor system 120 may not be able to recover anywhere near the amount of curated model data 108 or anywhere near the amount of empirical data obtained from reference bioreactor 110. In other words, local conditions may not yield the amounts of measurable data that is available to and deployed in the construction of reference bioprocess model 106. These limitations are understood to include those that are due to the intrinsically lower performance of measuring devices 122 of sensor system 120.

In light of the above, the bioprocess inside local bioreactor 102 is expected to yield measured redox data 124 that correspond to only a subset of model redox data 112. In many practical embodiments, measured redox data 124 may be significantly smaller than a full set of model redox data 112 yielded by reference bioprocess model 106. In some embodiments, the amount of measured redox data 124 is vastly smaller than the full set of model redox data 112.

Local learner 118 (or the unit on which local learner 118 is implemented) can be connected to an actuator system 126. Actuator system 126 interfaces with local bioreactor 102. Actuator system 126 deploys one or more individual actuators or input mechanisms 128 to control, provide inputs or, in any other way, alter or adjust the bioprocess transpiring in local biological entity 101 housed in local bioreactor 102.

In the exemplary embodiment of FIG. 1A a number of actuators 128 are deployed to adjust the bioprocess. Only actuators 128A and 128Z, here an input or inlet pipe and a stirrer, are expressly called out for reasons of clarity. It is noted that in some embodiments actuator system 126 may only utilize a single actuator or input mechanism, e.g., just inlet pipe 128A (or multiple inputs or inlet pipes, coupled to multiple sources of inputs—not shown) to supply additional quantities of biological entity 101 or other inputs. These other inputs could include other feed stock or materials, including, e.g., redox influencers. Alternatively, actuator system 126 can recommend an operation to a local operator (not shown).

Local learner 118, as shown, is connected to master learner 114 and configured to receive at least a portion of model redox data 112 from reference bioprocess model 106. For visualization purposes, a portion of model redox data 112 may be referred to as just a portion and will be designated by reference 112′. It is understood that in some embodiments, portion 112′ may include the full set of redox data 112. For example, portion 112′ could include the full or almost full set of model redox data 112 when local learner 118 is deployed with ample computing resources and disposes of significant communication bandwidth for receiving data.

Local learner 118 also receives the full set of measured redox data 124 obtained from local bioreactor 102 in which biological entity 101 is undergoing the bioprocess of interest. In other words, all measured data collected by measuring devices 122A-Z of measurement or sensor system 120 are supplied to local learner 118.

Meanwhile, portion 112′ of model redox data 112 supplied to local learner 118 from master learner 114 is accompanied by observable basis of redox indicators 116. This means that local learner 118 not only receives portion 112′, but also a mathematical basis in which to review both portion of model redox data 112′ as well as measured redox data 124. This is an advantageous aspect of the invention, since observable basis 116 allows learning system 100 to use a common evaluation measure or metric. Specifically, basis 116 is important for learning from portion 112′ provided for the bioprocess from reference bioprocess model 106 and measured redox data 124 collected from local bioreactor 102 in which biological entity 101 is undergoing the bioprocess.

In the embodiment of FIG. 1A, learning system 100 deploys a distributed learning algorithm 130 to learn. In the illustrated embodiment, distributed learning algorithm 130 resides in master learner 114 and in local learner 118. A person skilled in the art will realize that algorithm 130 can be further distributed among the resources of learning system 100. In fact, a module or part of distributed learning algorithm 130 can also reside within reference bioprocess model 106, as indicated in dashed lines in FIG. 1A. Such distribution can improve the efficiency of the learning process.

In any event, it is important that distributed learning algorithm 130 have access to model redox data 112 and measured redox data 124. By virtue of its distribution between at least master learner 114 and local learner 118 this condition is facilitated. Distributed learning algorithm 130 also has access to observable basis of redox indicators 116 picked or established by master learner 114 from model redox data 112 yielded by reference bioprocess model 106. Supplied with these, distributed learning algorithm 130 of learning system 100 can fulfill its main task. That task is to learn an optimal composition of redox data that should be measured under local conditions. In other words, the objective is to choose what measured redox data 124 is to be collected from the local bioprocess that biological entity 101 is experiencing in local bioreactor 102.

The ability to jointly evaluate locally collected redox data and model redox data, i.e., measured redox data 124 and model redox data 112 or just portion of model redox data 112′ in a common observable basis 116 is important. This joint evaluation enables distributed learning algorithm 130 to learn the optimal composition of measured redox data 132 that should be measured by sensor system 120 according to the method of the present invention. To illustrate this point, an optimal composition of measured redox data 132 described in basis 116 is indicated in FIG. 1A.

Optimal measured redox data 132 is shared between master learner 114 and local learner 118. A person skilled in the art will realize that any distribution and updating to optimal measured redox data 132 can be implemented by learning algorithm 130 anywhere in learning system 100. Indeed, once the learning is complete, local learner 118 could request from sensor system 120 to not measure all possible measured redox data 124 but only the redox data that are optimal 132 and expressed in basis 116. This approach helps to reduce the load on constrained local resources available to local learner 118.

Of course, even prior to discovering optimal measured redox data 132, master learner 114 preferably provides the reference learning model included in reference bioprocess model 106 to local learner 118. The model preferably contains a preliminary indication of optimal measured redox data 132 given context and local conditions. Supplying this information directly to local learner 118 at the very start or in an initialization step allows local learner 118 to train faster with less processing power or time. Meanwhile, learning algorithm 130 will converge on optimal measured redox data 132 to share between master learner 114 and local learner 118.

Once optimal measured redox data 132 are known, reference bioprocess model 106 can be updated. This is illustrated in FIG. 1A by an update protocol 134 that is sent from master learner 114 to reference bioprocess model 106. It should be understood that the update to reference bioprocess model 106 can also result in adjustments to curated model redox data 108. Such update could also lead to adjustments in reference bioprocess being run in reference bioreactor 110. This would be done in practice by changing the input(s) supplied through input 109 and sampling different output(s) drawn through output 111.

Before turning to the operation of learning system 100 it is important to appreciate the many types of local conditions and contexts in which it can be deployed. Most importantly, learning system 100 is not limited to bioprocesses transpiring in bioreactors. It is also not limited to one or just a few local biological entities. Learning system 100 is actually very well configured to applications in which many different biological entities in different contexts or under different local conditions are undergoing the bioprocess of interest. To better appreciate that applicability of the method and learning system 100 of the invention under these conditions we now turn to FIG. 1B.

FIG. 1B shows how learning system 100 is deployed when there are several local biological entities represented by living organisms. Local biological entities are live human subjects 201. Only some important body parts of four subjects 201A, 201B, 201C, . . . 201Z are shown for reasons of clarity. The reference numbers from FIG. 1A are retained in FIG. 1B to designate corresponding and/or analogous parts. Once again, the bioprocesses of interest involve reduction-oxidation reactions. The basics of redox reactions have already been discussed above in conjunction with the diagram of FIG. 1A.

In the configuration of learning system 100 shown in FIG. 1B, system 100 learns from reference bioprocess model 106 that is constructed form model redox data 108 and from model redox data 152 obtained from a reference biological entity 150. Again, reference bioprocess model 106 is understood to include an initial or reference learning model. Reference biological entity is a live human reference subject 150 undergoing the bioprocess of interest in a controlled environment; here under lab conditions. In the lab, model redox data 152 and other relevant parameters are easy to measure by the available measurement apparatus and systems 153. Thus, the bioprocess of interest can be treated as an empirical bioprocess under model conditions.

Alternatively, human reference subject 150 can be placed under model conditions that specifically correspond to local conditions. This is advisable whenever local conditions are expected to have a large influence on redox data or deviate substantially from lab conditions.

Model redox data 152 collected from reference subject 150 is used in generating the full set of model redox data 112. Model redox data 152 from reference subject 150 are further corroborated by curated model redox data 108. Both curated and model redox data 108, 152 are thus used in deriving full set of model redox data 112 for reference bioprocess model 106 and its reference learning model. Curated model redox data 108 can take into account mass spectrometer results resolving as many as 20,000 or even 50,000 potential peaks to locate known redox indicators for the bioprocess of interest. This can be accomplished by using a high-resolution mass spectrometer in which m/z for each ion is measured to several decimal places to differentiate between molecular formulas having similar masses. Suitable mass spectrometers include instruments supplied by commercial manufacturers such as Bruker, Sciex and others. Thus, in most cases, model redox data 108, 152 will far exceed the any measured redox data that can be collected under local conditions.

From reference bioprocess model 106 the full set of model redox data 112 is sent to mater learner 114. Master learner 114 is again shown connected with local learner 118. However, unlike in the embodiment of FIG. 1A, in the embodiment of FIG. 1B the individual connections between master and local learners 114, 118 are replaced by a primary feedback loop 154. Primary feedback loop 154 contains all of the connections required for master learner 114 and local learner 118 to communicate and for distributed learning algorithm 130 to learn efficiently. A person skilled in the art will realize that the connections in FIG. 1A can also be adapted to enforce the conditions of primary feedback loop 154, if desired.

Primary feedback loop 154 is used to communicate the relevant portion of model redox data 112′ from master learner 114 to local learner 118. Loop 154 is also used to communicate measured redox data 124 from local learner 118 to master learner 114. More importantly still, loop 154 is used to communicate changes or adjustments to the content or type of measured redox data 124 between learners 114, 118 under the direction of distributed learning algorithm 130. In other words, determination of optimal measured redox data 132 and its expression in basis 116 are arrived at by the use of primary feedback loop 154. The details of these adjustments will be discussed further below.

In embodiments where measured redox data 124 contains only observable redox indicators and/or candidates for such observable redox indicators, primary feedback loop 154 can interface directly with local measurement and control instruments. Thus, primary feedback loop 154 can be advantageously configured in some embodiments for adjusting the redox indicators in observable basis 116.

We now turn to local biological entities embodied this time by live human subjects 201. Only four particular subjects 201A, 201B, 201C and 201Z are shown experiencing the bioprocess of interest under their own local conditions 202. Once again, only local conditions 202A, 202B, 202C and 202Z of corresponding subjects 201A, 201B, 201C and 201Z are explicitly shown for reasons of clarity. Preferably, local conditions 202A, 202B, 202C and 202Z are simply the conditions under which subjects 201A, 201B, 201C and 201Z live day to day. In other words, local conditions 202A, 202B, 202C and 202Z are field conditions that match those of natural environments or habitats of subjects 201A, 201B, 201C and 201Z, respectively.

Local learner 118 may be implemented in a lower-power and/or lower bandwidth hardware unit such as a low-cost computer or tablet (not shown). The bandwidth and power comparison of the low-cost computer is made here with that of the measuring and processing capabilities of instruments available in the laboratory where human reference subject 150 is measured to yield model redox data 152 for reference bioprocess model 106.

In addition to running on the low-cost computer or local computing device, local learner 118 can be distributed over individual local learning units or devices 118A, 118B, 118C, . . . , 118Z residing in the corresponding local contexts 202A, 202B, 202C, . . . , 202Z of subjects 201A, 201B, 201C, . . . 201Z. Units 118A, 118B, 118C, . . . , 118Z may be embodied by a local computing device or affordance that may in some cases be connected and have access to cloud computing resources (but may be still constrained in comparison to reference and master learner resources). Thus, units 118A, 118B, 118C, . . . , 118Z can range from dedicated local devices, such as health monitoring apparatus, to standard local devices such as personal computers, mobile computing platforms (e.g., smart phones) as well as smart watches and even smaller wearable or stationary devices which may or may not be connected to additional cloud computing resources. In some cases, local learning units 118A, 118B, 118C, . . . , 118Z may have sufficient on-board computing resources to run a local portion of distributed learning algorithm 130. In some embodiments, local portion of distributed learning algorithm 130 is an application (app) that is downloaded and installed on the corresponding local unit.

Under local conditions 201 experienced by subjects 202 the test or sensor system deployed may again be a significantly down-scaled version in comparison to the test or sensor systems available in a laboratory where the reference human subject 150 is tested. Still, in some cases, the ability of local sensor system to capture measurement data under local conditions may be quite high. For some specific measure or redox indicator, the local capability may even be higher.

The sensor system as a whole is not explicitly shown in FIG. 1B. Instead, we see here individual sensors or measurement devices 122 deployed in local contexts or under local conditions 202 of subjects 201. In the illustrated embodiment, all measurement devices 122 are shown as being different and are configured to collect different measurements. Of course, they could also be configured to collect measurements of the same observable redox indicator or parameter from several or all subjects 201.

As shown, distributed local learning units 118A, 118B, 118C, . . . , 118Z are assigned to their subjects 201A, 201B, 201C, . . . , 201Z and connected to corresponding specific measurement devices 122A, 122B, 122C, . . . , 122Z within local contexts 202A, 202B, 202C, . . . , 202Z. Each one of measurement devices 122A, 122B, 112C, . . . , 122Z, as shown, is configured to collect one or more types of measured redox data 124. Relevant redox data that should be measured can fall into any one or more of the five categories of redox data discussed above.

In the illustrated example, measurement device 122A is a wrist band in wireless communication with local learning unit 118A. Wrist band 122A can measure, pulse, blood oxidation level (optically) and blood pressure of human subject 201A. These types of measurements can yield measured redox data 124A that is direct and immediately available to learning algorithm 130. Other measurements that can be obtained from wrist band 122A include activity or exercise measurement from accelerometers and other sensors, respiration or other respiratory measures, heart rate and its variability, hydration or concentrations of fluids, photo or image data of the subject, such as skin or other parts, and other diet or lifestyle-related measurements.

Measurement device 122B, as shown, is a blood sampler connected directly to local learning unit 118B. Blood sampler 122B can draw blood and/or plasma for measurement of any redox indicator. Preferably, blood and/or plasma measurements are performed under local conditions 202B as soon as the blood and/or plasma are drawn. Kits containing sensors and analysis instruments that can be used as measurement device 122B are marketed by a number of commercial suppliers. Blood glucose testing devices from Roche, Abbott, Johnson & Johnson and other suppliers are widely available. Another example includes the home blood test kit from COR that measures HDL cholesterol, LDL cholesterol and total cholesterol, fasting blood glucose, inflammation markers such as fibrinogen and triglycerides. Another example includes hand-held blood test kits from CardioChek that can measure total cholesterol, HDL cholesterol, triglycerides and glucose. Still another example includes ketone testing kits with the Precision Xtra Blood Ketone Monitoring System and combined ketone and blood monitoring systems using the MultiSure GK Blood Glucose & Ketone Monitoring System from Apex Biotechnology Corp. Other examples require devices or samples to be mailed to the lab or for the subject to visit a clinical lab. These are, however, available directly to consumers and include the saliva, blood spot, serum and urine test kits from ZRT Laboratory, the food and chemical sensitivity test kits from Cell Science Systems, and the blood tests provided by clinical labs such as Quest and Labcorp through various direct-to-consumer suppliers including HealthLabs.com and Walk-In-Lab. In many cases, devices 122B that are chosen can reduce hemolysis and autoxidation of the blood (e.g., by proper collection technique(s)) and reduce collection artifacts in plasma (e.g., by using antioxidants and alkylating agents during plasma collection) of subject 201B. They are preferably also able to perform rapid local measurement(s). Thus, measured redox data 124B is made available to learning algorithm 130 with minimal delay.

Measurement device 122C, as shown, is a urine sampler that connects to local learning unit 118C. Urine sampler 122C collects urine from subject 201C for any measurement of a redox indictor that can be made thereon. Preferably, the measurements are performed under local conditions 202C as soon as the urine sample is collected. As in the case of blood and plasma testing, there are kits (home kits or field kits) containing sensors and analysis instruments that can be used as measurement device 122C. Devices in such kits have the ability to perform immediate measurement on the urine of subject 201C to make measured redox data 124C available to learning algorithm 130 with minimal delay. That is because the results can be observed visually from a test strip and entered manually by subject 201C or read automatically by a reader associated and/or coupled with measurement device 122C. Examples include reagent strips such as HealthyWiser Urinalysis Reagent Strips that test urine for glucose, protein pH, leukocytes, nitrites, ketones, bilirubin, blood, urobilinogen and specific gravity.

There are many additional home or field kits with measurement devices capable of collecting still other measurements. These include devices that can collect samples of saliva, serum, skin as well as bodily fluids including excretions and secretions. Further examples include blood spot testers and analyte tests ranging from paper chromatography to electrochemical sensors. To the extent that the measurement can provide measured redox data, i.e., data that is related to the redox status of the bioprocess of interest, such measurement devices are considered suitable in the context of the present invention. It is understood that a wide range of measurement devices 122 can be directly or indirectly connected to local learner 118. Measurement devices 122 may produce data that is transmitted to the system via an application program interface from another database or monitoring system, or connected by means of a file export from another device or system and then imported into the system accessed by the local learner, or other data output is in a format that can be optically scanned, manually entered, or a combination of methods to provide measurement data to the system accessed by local learner 118.

Measurement device 122Z in the present example is shown as a wrist-worn, integrated personal health monitor. In alternative embodiments, measurement device 122Z can be embodied by a personal health monitor in another format including a wearable patch, a wearable device on a location other than the wrist, an implantable device or a device with an implantable or subdermal component, an ingestible or insertable device, or a portable or hand-held device.

As shown, health monitor 122Z is in communication with learning unit 118Z via any suitable communication link. In the present case, the communication link is wireless, as indicated. Health monitor 122Z measures the daily activities of subject 201Z. These may include the number of steps taken, the relative rigor of exercises performed, amount of sleep, calories consumed, and the like. Persons skilled in the art will be familiar with all possible measurable quantities that can be collected with and without the assistance of subject 201Z. Note that direct input by subject 201Z in either prompted or unprompted self-reporting is also considered a measurement.

Each local subject 201 undergoing the bioprocess under their own local conditions 202 generates measured redox data 124 for the bioprocess. Specifically, local subject 201A generates measured redox data 124A. Local subject 201B generates measured redox data 124B. Local subject 201C generates measured redox data 124C. Finally, while under their local conditions 202Z, local subject 201Z generates measured redox data 124Z.

Measured redox data 124A, 124B, 124C, 124Z is passed via distributed local learners 118A, 118B, 118C, . . . , 118Z to local learner 118. There, the combined measured redox data 124 is communicated from local learner 118 to master learner 114 using primary feedback loop 154. It should be noted that even all measured redox data 124 is usually just a small subset of model redox data 112 on which reference bioprocess model 106 is based.

In addition to measurement devices 122 of local sensor system, learning system 100 is shown as including an actuator system for providing inputs, changing, altering or adjusting the bioprocess experienced by local subjects 201. In the embodiment of FIG. 1B, actuator system has individual actuation mechanisms 128 provided for each local subject 201 in their corresponding contexts 202. Mechanisms 128 can be used by subjects 201 to self-administer or receive the requisite adjustment, action or prompt. In principle, mechanisms 128 may also administer actions or adjustments without the participation or awareness of local subjects 201. For example, such situations may arise when one of the local subjects 201 is under active care and their context 202 may be a home care facility. Mechanisms 128 can also include drug delivery devices, an insulin pump, an oxygen-providing device, a device that changes a medication or food formulation automatically, a device that alerts a patient to take medication or some other input, or a device that recommends a change to medication, food, nutritional supplement or any other aspect of a subject's regimen.

FIG. 1B illustrates four exemplary mechanisms 128 belonging to the actuation system. In context 202A mechanism 128A is embodied by a vitamin and supplement pill dispenser. The dosage of vitamins and supplements from dispenser 128A can be adjusted by communicating the dosage to subject 201A upon review of their measured redox data 124A and based on the learning as described below. Alternative embodiments include automating the adjustment or recommendation to an operator to adjust the formulation of vitamins or supplements or their delivery to subject 201A.

In context 202B mechanism 128B is embodied by a syringe for drug self-administration by subject 201B. Once again, the time and dosage for subject 201B is determined upon review of their measured redox data 124B and based on the learning performed by learning system 100, as described below. Alternative embodiments include automating or recommending the adjustment to the formulation of medications, medical foods or nutritionals for self-administration by subject 201B, administration or oversight by an informal or professional caregiver, or administration by an automated delivery system or device.

In context 202C mechanism 128C is embodied by a clock. Clock 128C may be provided with appropriate alarms, chimes, reminders or other prompts that can remind subject 201C or a caregiver or proxy about important actions to take. For example, clock 128C may be set up to remind subject 201C about urine sample collection time. In addition, clock 128C can be set to provide other reminders, e.g., to conduct certain prescribed or therapeutic activities.

In the case of subject 201Z under local conditions 202Z, actuation mechanism 128Z is integrated with measurement device 122Z. Specifically, the display of health monitor 122Z is configured to visually communicate to subject 201Z an action or adjustment that should be undertaken. As above, the adjustment or action are dictated by learning from measured redox data 124Z collected from subject 201Z under local conditions 202Z. The adjustment or action may be automatically undertaken by a device, recommended to a subject or a caregiver of the subject.

### General Principles of Operation of Learning System

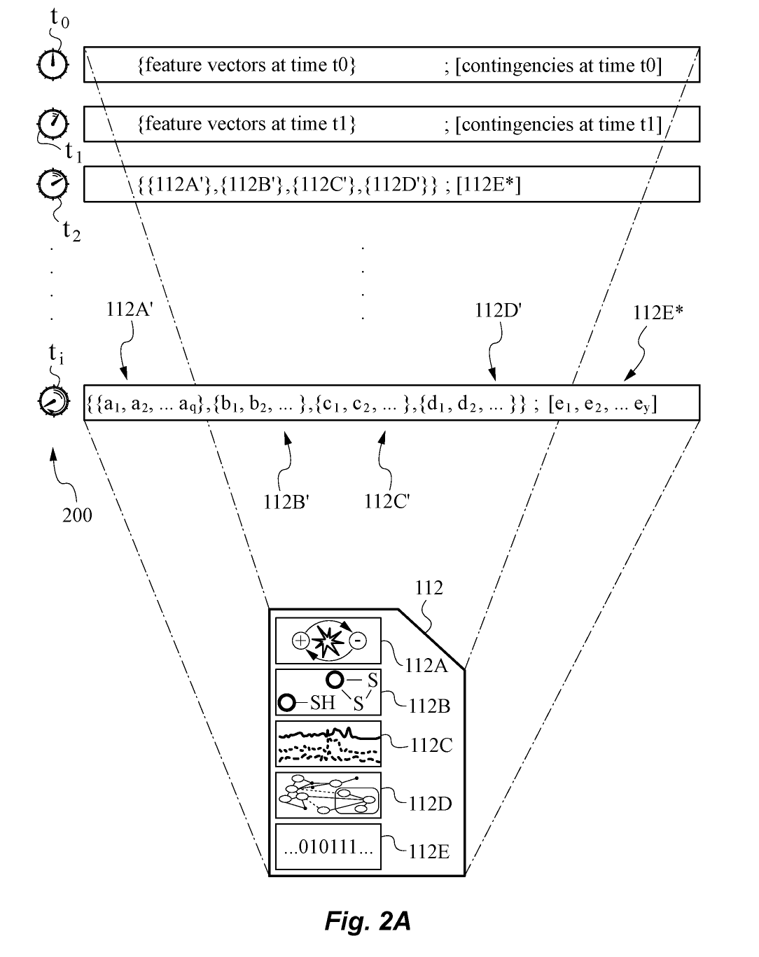

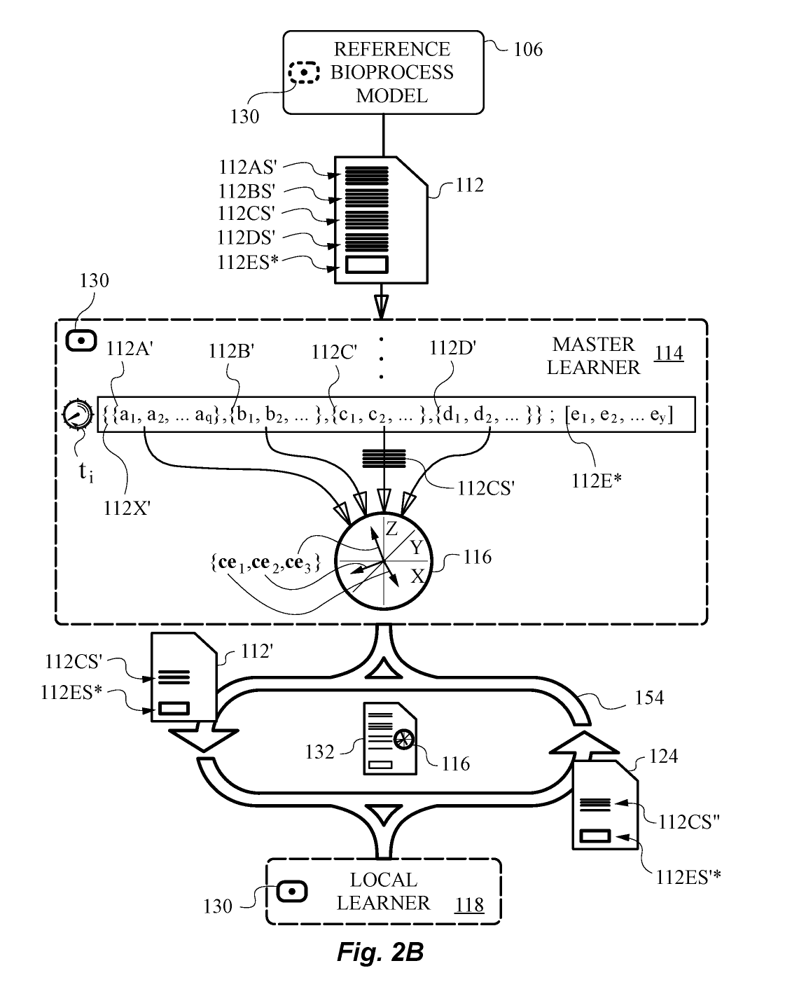

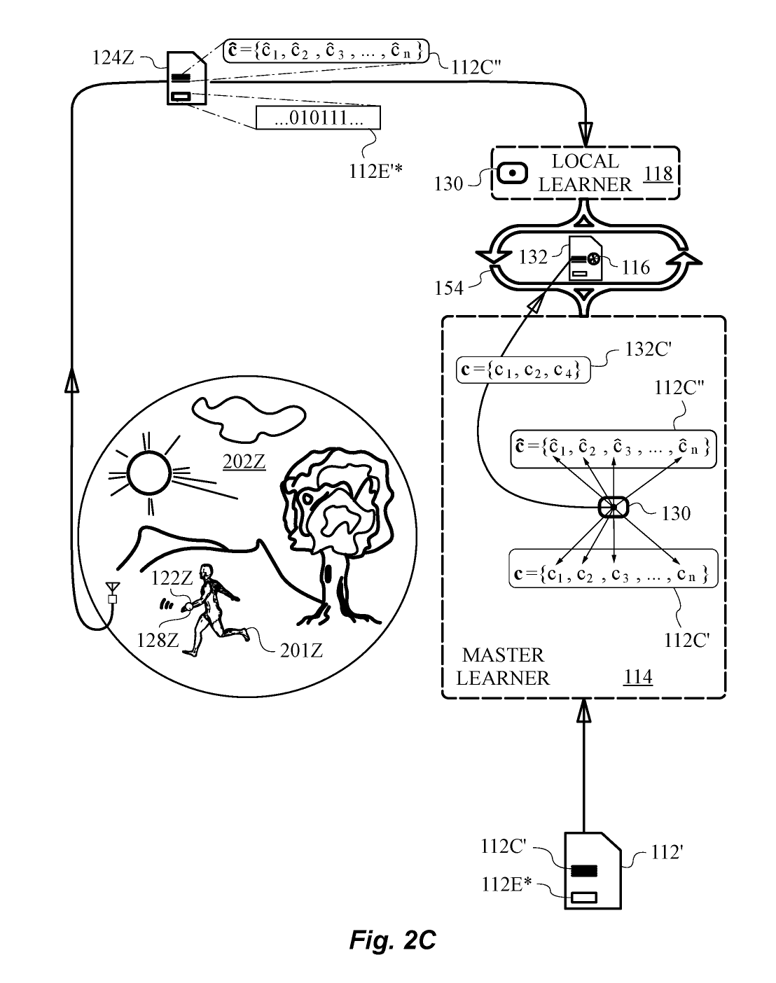

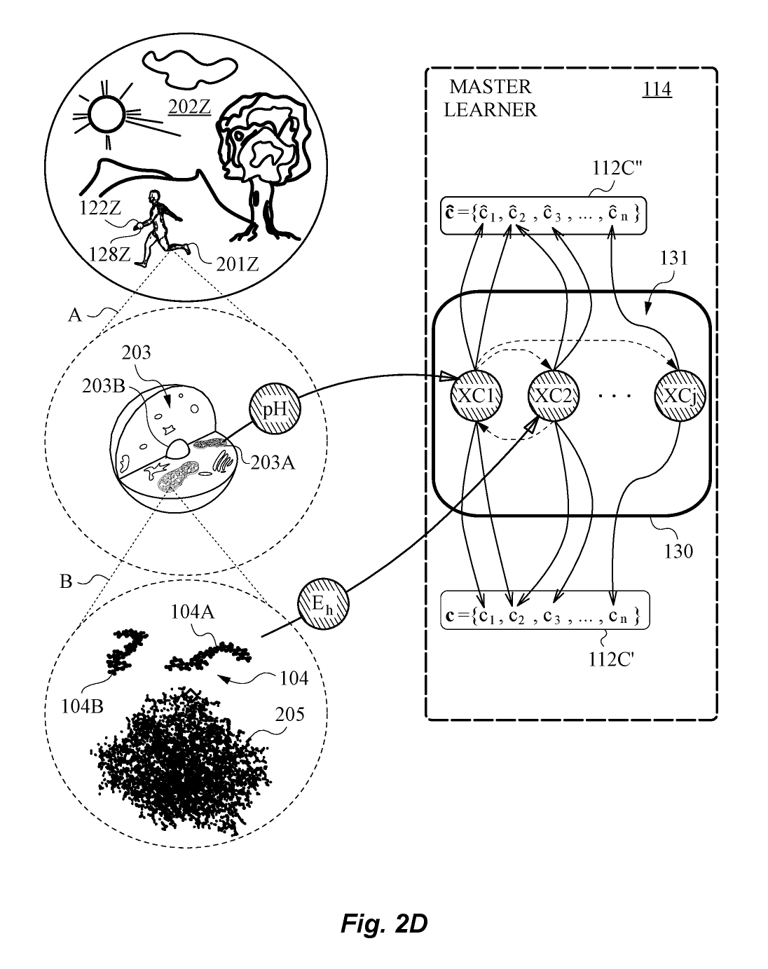

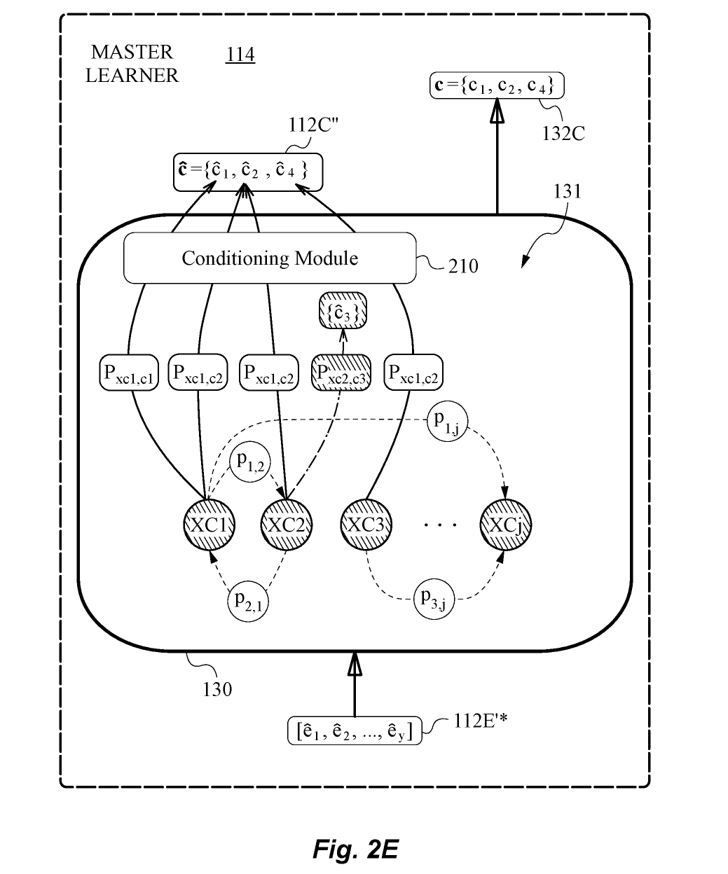

Having reviewed two high-level embodiments of learning system 100 as shown in FIGS. 1A and 1n FIG. 1B we now turn to the operation of distributed learning algorithm 130 and the format of redox data. Specifically, we turn to FIGS. 2A-B to examine an advantageous representation of model redox data 112, measured redox data 124, portion of model redox data 112′ (sent from master learner 114 to local learner 118; see FIGS. 1A-B) and optimal measured redox data 132.

FIG. 2A is a diagram illustrating model redox data 112 for the bioprocess of interest provided by reference bioprocess model 106 (see FIGS. 1A-B). As noted above, model data 112 can contain redox data fitting into any of the five different categories of redox data. Namely it can contain redox data that fits into any one or more of the four redox code categories 112A-D by which biological systems are organized. Model data 112 can further contain redox data that fits into a fifth category of contingent redox data 112E.

In many of the embodiments the most important categories may include the first, third and fifth. These include bio-energetics redox data 112A, signaling redox data 112C and contingent redox data 112E. The fifth category typically includes candidates for any of the first four categories and data about local conditions and model conditions; i.e., contextual data. Contingent information can also include data about items that are not directly measurable, i.e., “soft data”, and any other contingent data including speculatively related information. Some information that is not directly measurable can be placed in the category of candidate data for which further statistical analysis may later discover an association. Although the first, third and fifth categories of redox data 112A, 112C, 112E will be most important in most embodiments we are concerned about herein, we consider all five categories of redox data 112A-E for reasons of completeness.

FIG. 2A expands and visualizes an entire set of model redox data 112 yielded by reference bioprocess model 106. We first consider model data 112 at a particular time t₁ indicated by a running clock on the left side of the drawing figure for clarity. At time t₁ model redox data 112 is shown partitioned into generalized feature vectors 112A′-112D′ and a contingency list 112E*. The prime and star notation is used to indicate that the five categories of model redox data 112 contain structured data, here represented as vectors, in the first four categories and a list of generally unstructured data in the fifth category. Of course, candidate features for feature vectors 112A′-112D′ are technically structured data. Meanwhile, purely contextual data such as annotations and labels is typically unstructured but may affect how structured data should be treated. For example, contextual data may indicate in which contexts no data in any of the first four categories is even expected to relate to the bioprocess of interest.

Specific data entries, such as elements, features or other data falling into categories of bio-energetics redox data 112A, switching redox data 112B, signaling redox data 112C and network redox data 112D are incorporated into correspondent feature vectors 112A′, 112B′, 112C′, 112D′ representing redox data in these categories. Thus, data entries ranging from 1 to q and designated by a₁, a₂, . . . , a_{q} falling into the category of bio-energetics redox data 112A become entries in feature vector 112A′. Similarly, data entries b₁, b₂, . . . as well as c₁, c₂, . . . and d₁, d₂, . . . belonging to the other three redox data categories become entries in feature vectors 112B′, 112C′ and 112D′, respectively. Meanwhile, redox data in the fifth category 112E containing candidates, contextual and other subject-related and unstructured data is represented in list 112E*. In other words, as illustrated, no further data representation, format or structure is imparted on redox data 112 belonging to fifth category 112E.

As is made clear in FIG. 2A, model redox data 112 is not only subdivided by category but is further ordered in a time sequence 200. Particular instants in time sequence 200 are denoted by the status of the running clock drawn on the left side in the figure. Only start time, t₀, times t₁, t₂ and a certain time of interest t₁ are indicated explicitly. However, given that all bioprocesses of interest transpire in time, reference bioprocess model 106 contains the time parameter to describe the unfolding of the bioprocess and provides model data 112 within the framework of time, or in terms of time sequence 200. The formulation of model redox data 112 at times t₀, t₁, t₂ shows in a more compact manner a convenient formatting for use in learning system 100 and distributed learning algorithm 130 (see FIGS. 1A-B). For an unchanging or steady state, the redox status, and hence the corresponding model data 112, do not change with time. The time parameter can be left out when dealing with persistent or steady state redox status, or when the output of the learning process is a classification or other result that is not part of a dynamic process with a feedback loop and control.

FIG. 2B illustrates master learner 114 receiving from reference bioprocess model 106 model redox data 112 formatted as feature vectors 112A′, 112B′, 112C′, 112D′ and as list 112E*. Only model data 112 at time of interest t₁ is shown explicitly for reasons of clarity. This simplification will allow us to better understand how model data 112 is treated by master learner 114.

In accordance with the invention, master learner 114 is configured to receive model redox data 112 and establish therefrom the observable basis of redox indicators 116. List 112E* is not typically used in establishing observable basis 116. That is because in addition to potential redox indicator candidates in structured data, it also contains unstructured data about contexts, annotations and labels on redox data and any other types of data related to one or more redox categories. As with any machine learning process, list 112E* may contain data that do not associate with the state being inferred through the learning process executed by distributed algorithm 130. Such data may drop out of the regression through methods such as principal components analysis. However, time series 112ES* of lists 112E* at times t₁, t₂, . . . , t₁ is nonetheless provided to master learner 114 so that it can make the determination whether or not to drop any data from lists 112E*. Master learner 114 can also make a determination to drop other measurement data that turns out not to be a principal component with respect to the learning model. Meanwhile, data entries in each of the feature vectors 112A′, 112B′, 112C′, 112D′ are used by master learner 114 to estimate corresponding redox category vector spaces. More precisely, time series 112AS′, 112BS′, 112CS′, 112DS' of corresponding feature vectors 112A′, 112B′, 112C′, 112D′ are used for estimating the corresponding vector spaces using the standard tools of linear algebra and applied mathematics. These include testing for inner products to establish orthogonality, determining vector norms and other tests known to the skilled artisan. Among other, as illustrated, the results yield the dimensionality of the corresponding vector spaces and a measure of their stability.

Preferably, reference bioprocess model 106 provides provisional suggestion about the vector spaces of feature vectors 112A′, 112B′, 112C′, 112D′. These may be based on model data 108 and data from reference bioreactor 110 or reference subject 150, depending on the context. However, because of the limitations under local conditions, available measurement devices as well as contextual factors, master learner 114 needs to re-validate the vector spaces to ensure minimal stability and norm preservation to enable the implementation of learning algorithm 130. Persons skilled in the art will be familiar with many different methods for setting such bounds. Master learner 114 can take advantage of any of these prior art methods in ensuring the requisite stability of the vector spaces for effective machine learning.

One of the challenges of inferring the redox state in any of the four redox categories is that some compartments of biological entity 101, whether a biomass or a living subject (such as human subject 201, see FIG. 1B) are parts of highly redundant pathways with multiple uses. The redundancy of the pathways is the product of evolutionary pressures. The redundancy and many branching points may often present to a learning algorithm as cross-talk, fading, noise and other effects. These may be taken into account when estimating separate vector spaces for the four types of feature vectors 112A′, 112B′, 112C′, 112D′.

As shown in FIG. 2B, feature vectors 112A′, 112B′, 112C′, 112D′ of all four redox categories containing structured data can be collapsed into one joint feature vector 112X′. This simplification may be necessary under some local conditions and/or if the measurement device(s) are not capable of yielding information that clearly fits into the first four redox categories. This simplification may also be used if the vector spaces for feature vectors 112A′, 112B′, 112C′, 112D′ are not sufficiently stable, there is a high level of cross-talk between them and/or the environment, fading, aliasing or any other source of artifacts or noise. The rules that apply distributed machine learning algorithm 130 to joint feature vector 112X′ are the same as in the case of any one or more of the four redox categories. Note that joint feature vector 112X′ will generally be higher-dimensional than any one of feature vectors 112A′, 112B′, 112C′, 112D′.

The need for collapsing feature vectors 112A′, 112B′, 112C′, 112D′ to single joint feature vector 112X′ due to the above-mentioned limitations stems from the real-world, as this inherent noisiness of even model redox data 112 will often be present. Biological entities have evolved redundancies to enable them to survive a wide range of environmental stresses. This creates the challenge that it is therefore difficult to measure and attribute any specific redox indicator to a specific process—e.g., to any specific type of oxidative stress that is exemplified by the bioprocess of interest.

For this reason, among others, learning system 100 attempts to identify the optimal features or redox indicators that can serve as a fingerprint for redox status through distributed learning algorithm 130 and the available learning techniques. Redox status in a hidden compartment is difficult to measure, and is hence treated as hidden. In fact, any such individual measure may be too non-specific to yield meaningful results. However, the present learning algorithm 130 focuses on patterns in measurement redox data including select observable redox indicators that, when taken together with additional available context redox data in the fifth redox category, can yield useful inferences with respect to redox status.

Still in reference to FIG. 2B, we focus on redox category three of signaling redox data 112C as an example to provide a detailed explanation of the workings of learning algorithm 130. A person skilled in the art will recognize that the example of signaling redox data 112C represented in feature vectors 112C′ applies to redox data in any of the first four categories that contain structured redox data. In fact, the manner of dealing with a joint feature vector into which two or more feature vectors 112A′, 112B′, 112C′, 112D′ are collapsed if necessary, would be analogous. Thus, the following description for feature vector 112C′ applies just as well to joint feature vector 112X′.

Reference bioprocess model 106 transmits time series 112CS' of feature vectors 112C′ collected at times t₁, t₂, . . . , t₁ from reference biological entity 110 or 150 and/or validated and corroborated with curated model data 108 (see FIGS. 1A-B) to master learner 114. In some cases, times t₁, t₂, . . . , t₁ are selected in reference bioprocess model 106 to mark distinct stages, transitions, reaction periods or still other important times in the bioprocess of interest. Each feature vector 112C′ in time series 112CS' that is not steady state exhibits different values in data entries {c₁, c₂, . . . , cₙ}. The entries are taken to range from 1 to n (i.e., there are n data entries in feature vector 112C′). In order to be suited for machine learning, each one of data entries {c₁, c₂, . . . , cₙ} is preferably an accepted observable redox indicator, as mentioned above.

Redox balance is due to relative oxidation/reduction status between redox couples operating at the physical chemistry level. Some of the most suitable couples without their co-factors are listed in Tables 1A-C below.

TABLE 1A
Redox Pairs
* Isotopically Labeled Standard used

| | Analytes |
|---|---|
| Panel 1 | Cystine* |
| | Cysteine* |
| | Cysteine Persulfide* |
| | GSSG* |
| | GSH* |
| | GSH Persulfide* |
| | HomoCystine* |
| | XOMA |
| | H₂S* |
| | Thiosulfate* |
| | Tetrathionate |
| | CysGly Dipeptide* |
| | GluCys Dipeptide* |
| | Cys-GSH Disulfide |
| | Ophthalmic Acid* |
| | Cystathionine |
| | Lanthionine |
| | GSH-Sulfonic Acid |
| | Lipoic Acid |
| | Cysteamine |
| | Methionine* |
| | Adenosine* |
| | SAM* |
| | SAH |
| | Spermine* |
| | Spermidine* |
| | Citrulline* |
| | Ornithine |
| | Kynurenine |
| | Kynurenic Acid |
| | Serine |
| | Taurine* |
| | Pyroglutamic Acid |
| | α-Aminobutyric Acid* |
| | 3-NitroTyrosine* |
| | 3-ChloroTyrosine* |
| | Glutamate |
| | Homocitrilline |
| | Aspartate |

TABLE 1B
Redox Pairs
* Isotopically Labeled Standard used

| | Analytes |
|---|---|
| Panel 2 | NAD |
| | NADP |
| | AMP |
| | ADP |
| | ATP |
| | cAMP |
| | Xanthine |
| | Hypoxanthine* |
| | 2-deoxy-guanosine* |
| | Inosine |
| | Acetyl-Carnitine* |
| | Carnitine |
| | NADH |
| | NADPH |
| | Urate |
| | 8-OH-dG |
| | Pyrimido purinone |
| | Fumurate* |
| | Succinate* |
| | Lactate* |
| | Pyruvate* |
| | Acetoacetate |
| | 3-Hydroxybutyric |
| | Acid |
| | 743-OH |
| | 743* |
| | 886 |
| | A0001-OH |
| | A0001* |
| | α-TOC |
| | α-CEHC |
| | δ-CEHC |
| | 743-OH-Sulfate |
| | 743-OH-Gluc |
| | A0001-OH-Sulfate |
| | A0001-OH-Gluc |
| | 589* |
| | 589-OH |
| | 589-Sulfate |
| | 589-Gluc |

TABLE 1C
Redox Pairs
* Isotopically Labeled Standard used

| | Analytes |
|---|---|
| Panel 3 | CoQ10 |
| | Ubiquinol (CoQ10-OH) |
| | Docosahexaenoic Acid (DHA)* |
| | Arachidonic Acid (AA)* |
| | Linoleic Acid |
| | Palmitoyl Carnitine |
| | Prostaglandin E2* |
| | tetranor PGE-M* |
| | tetranor PGA-M |
| | 15-Deoxy-PGJ2 |
| | 15-Deoxy-PGJ2-GSH |
| | Leukotriene E4* |
| | Leukotriene C4 |
| | 8-iso-PGF2a* |
| | Creatinine (urine) |
| | 2,3-DPG (RBC contamination of |
| | plasma) |

As discussed above, measures of actual redox balance between individual redox may be inaccessible in many contexts. Even if possible in principle, due to local conditions such measurements may not be feasible in many applications for which the presently described systems may be used. In other words, in some cases, measures of redox reactions at the level of physical chemistry may not be considered as candidates for observable redox indicators.

Of course, even though they may not be accessible, such redox reactions clearly do occur and would advantageously be accounted for in some manner. For this reason, any unobservable redox reactions or their consequences at the level of physical chemistry or higher are tracked herein as hidden states. Even though the real and observable basis of redox indicators will not include any hidden states or otherwise hidden or inaccessible data, their presence is expressly included in the learning model, as discussed below.

Particularly useful and established electron balance indicators that classify as observable redox indicators include the presence or concentration of an oxidoreductase or of an oxidoreductase co-factor. Other observable redox indicators include the presence or concentration of balance influencer compounds, electron balance influencer compositions or still other redox-active compounds. The reader is again referred to Tables A-C above for a partial list.

Still other observable redox indicators include, e.g., pK values, pH values, threshold values, context measures and soft indicators. Note that soft indicators will typically be placed among the contextual and other unstructured data in list 112E*. Data entries {c₁, c₂, . . . , cₙ} of each feature vector 112C′ contain one of the candidate observable redox indicators. Hence, each vector 112C′ can be written as:

112C′=c={c₁,c₂,c₃ . . . cₙ} [Eq. 1]

where boldface lower-case lettering is used to designate a vector quantity. The series 112CS' can then be described as a series of vectors c composed of observable redox indicators {c₁, c₂, . . . , cₙ} as set forth in Eq. 1.

The underlying rules of redox reactions in the corresponding redox category, here the redox signaling category, may dictate that as time progresses the selection of observable redox indicators {c₁, c₂, . . . , cₙ} exhibit a certain conservation pattern. For example, if observable redox indicators {c₁, c₂, . . . , cₙ} encode all participating elements or molecules in a relatively isolated redox signaling pathway, then their number should be conserved. Therefore, series 112CS' is expected to obey a certain conservation criterion. An example appreciated by the skilled artisan is the conservation of reagents irrespective of the individual fluxes (reactions) in stoichiometry. In other words, the total of entities at the start and at the end cannot change (also referred to as conservation of elements or constituents). This conservation law allows one to set up and deploy the well-known stoichiometric matrix S.

From a conservation criterion or other known rule a matrix equation, possibly involving stoichiometric matrix S or a transition matrix, can be set up. Once the matrix equation is set up, the vector space of vectors c can be parameterized and a set of linearly independent vectors that span that vector space can be established. When normalized, such vectors represent observable basis 116 for vectors c composed of observable redox indicators. In other words, any vector c can be obtained or decomposed in a linear combination of the vectors in basis 116.

In a preferred embodiment master learner 114 can receive initial guidance on a suitable basis 116 from reference bioprocess model 106. For example, the module of distributed learner 130 residing in reference bioprocess model 106 can be in charge of providing such initial suitable basis 116 as part of the reference learning model (described in more detail below). However, in many cases, this suggestion will be adjusted based on local conditions and what can be measured. For example, if only a small subset of redox indicators that model 106 is based on can be measured, then master learner 114 will have to reduce the dimensionality of basis 116. In applying the tools of linear algebra care needs to be taken to ensure a reasonable level of completeness, orthogonality and other requirements for applying the desired learning algorithm, as discussed below. It is duly noted that some of the observable redox indicators may be present in more than one redox category. In other words, observable redox indicators in feature vectors 112A′, 112B′, 112C′ and 112D′ may be the shared.

In some situations, overlap in observable redox indicators between redox categories leads to unacceptable levels of cross-talk for machine learning. In those cases, joint feature vector 112X′ should be used. As already stated, joint vector 112X′ simply combines available redox indicators into a single feature vector in a single or joint vector space. In situations where the cross-talk is acceptably low, the same process as in the case of feature vector 112C′ is followed for establishing bases in the vector spaces containing feature vectors 112A′, 112B′ and 112D′. In any case, master learner 114 can receive initial guidance from distributed learning algorithm 130 resident in reference bioprocess model 106 about the level of cross-talk to expect and whether combining the vector spaces is advisable.

FIG. 2B shows observable basis 116 for feature vectors 112C′ in third redox category consisting of basis vectors {ce₁, ce₂, ce₃}. Only three basis vectors are shown in this case for reasons of clarity. The vector space containing feature vectors 112C′ could and typically will have a higher dimensionality than 3. The vector spaces containing feature vectors 112A′, 112B′ and 112D′ also have basis vectors {ae₁, ae₂, . . . , ae_{q}}, {be₁, be₂, . . . , beₘ} and {ae₁, ae₂, . . . , aeₙ}, respectively. The dimensionalities of their vector spaces are equal to the numbers of entries or observable redox indicators, i.e., q, m and n. Basis vectors {ae₁, ae₂, . . . , ae_{q}}, {be₁, be₂, . . . , beₘ} and {ae₁, ae₂, . . . , aeₙ} are not shown explicitly in FIG. 2B for reasons of clarity.

When referring to observable basis 116 herein, we mean all basis vectors {ae₁, ae₂, . . . , ae_{q}}, {be₁, be₂, . . . , beₘ}, {ce₁, ce₂, ce₃} and {ae₁, ae₂, . . . , aeₙ} or any joint observable basis. Of course, observable basis 116 can be reduced to just one or a select few of the redox categories in applications where redox status corresponding to just the one or just the select few of the redox categories is being measured.

In addition to providing observable basis 116, master learner 114 also reduces the amount of model redox data 112 communicated to local learner 118 to just portion 112′ based on specific context and local conditions. In the simple case of only concentrating on redox data in the third category, master learner 114 can remove from the portion of model redox data 112′ all redox information in the first, second and fourth categories. In other words, feature vectors 112A′, 112B′ and 112D′ can be dropped by master learner 114 from portion 112′ that is sent to local leaner via primary feedback loop 154. Only time series 112CS' would thus be included in portion 112′. Furthermore, if the temporal resolution of measurement at the local end is low, then master learner 114 may further reduce the amount of data by sending only a sub-sample of time series 112CS′. Exactly this situation is illustrated in FIG. 2B, wherein portion 112′ contains only a sub-sample of time series 112CS' and does not contain any redox data in categories one, two and four.

In any particular embodiment, local learner 118 receives at least portion 112′ of model redox data 112 from reference bioprocess model 106. In addition to limiting portion 112′ based on relevancy, i.e., where portion 112′ contains only model redox data relevant to local conditions or is otherwise a limited portion of model redox data 112, master learner 114 can also limit it for other reasons. Such other reasons or considerations can include the bandwidth of primary feedback loop 154 and technical considerations, capabilities and throughput of sensors or measuring devices as well as other aspects of local conditions.

On the other hand, local learner 118 can receive all measured redox data 124 from local biological entity undergoing the bioprocess. Local learner 118 preferably shares all measured redox data 124 with master learner 114 via primary feedback loop 154. This situation is shown in FIG. 2B, where measured redox data 124 contains all measured redox data 124. The number of measured feature vectors 112CS″ (where double prime notation is used here and below to distinguish model from measured quantities) in measured redox data 124 is larger than in portion 112′ that is sub-sampled. It is preferable not to discard extra data if measurement devices or sensors under local conditions are capable of capturing it. A person skilled in the art of signal processing will appreciate how to best take advantage of additional information and headroom in sensor performance.

FIG. 2C is a diagram that focuses on measured redox data 124Z from subject 201Z as introduced in FIG. 1B. Of special interest is measured redox data in third redox category 112C. This redox data is structured and formatted as feature vector 112C″. The entries in measured feature vector 112C″ conform with the requirements of forming a proper vector in the vector space spanned by basis vectors {ce₁, ce₂, ce₃} (see FIG. 2B). The data entries in feature vector 112C″ correspond to the definition provided in Eq. 1 above. However, because each of the data values is obtained from a measurement, a “hat” is placed above it to denote that fact. This is standard notation for measured quantities frequently deployed by those skilled in the art. Measured feature vector 112C″ is thus written as:

112C″=ĉ={ĉ₁,ĉ₂,ĉ₃ . . . ĉₙ}.

The measured redox data series 112CS″ can then be described as a series of vectors ĉ, exactly as the series of model vectors c set forth in Eq. 1. Another way to express the temporal dependence of model and measured feature vectors is to introduce time explicitly—i.e., c=c(t) and ĉ=ĉ(t).

FIG. 2C also shows in more detail the local conditions 202Z under which human subject 201Z can be measured. Integrated measurement device 122Z and actuation device or mechanism 128Z are shown in the same wrist-worn health monitoring device that subject 201Z is wearing during their exercise routine. Local conditions 202Z at the level of subject 201Z are outdoors. The contextual information includes list data such as running, weather, elevation, prior subject data and any other information that is relevant to redox status. All the contextual information may then be provided in the fifth category of measured redox data 112E′*. List redox data 112E′* is part of measured redox data 124Z for subject 201Z.

Measurement device 122Z in health monitoring unit is shown using the wireless channel to transmit measured redox data 124Z to local learner 118. More specifically, it is the distributed portion of local learner 118A (see FIG. 1B) running as an application on health monitoring device that effectuates the wireless transmission. In this case local learner may be running on a dedicated computing device at the home of subject 201Z. Alternatively, local learner 118 can run on a computing device assigned to a group of subjects to which subject 201Z belongs. In that case local learner 118 can run on a computer at a health and fitness facility or a health monitoring establishment, including health care facilities. Again, in each case, local computing device could be a combination of a local device or local interface and cloud computing resources. A person skilled in the art will recognize that suitable options and communication architectures for transmitting measured redox data 124Z to local learner 118 are vast and should be chosen in accordance with standard protocols known to the skilled artisan.

FIG. 2C also shows master learner 114 and local learner 118 with learning algorithm 130 distributed between them. This distribution ensures that learning algorithm 130 has access to model redox data 112 arriving through master learner 114 and to measured redox data 124Z arriving through local learner 118. All the necessary communications between master and local learners 114, 118 are supported by primary feedback loop 154.

As illustrated, learning algorithm 130 has access to observable basis of redox indicators 116 for the third redox category, i.e., {ce₁, ce₂, ce₃, . . . , ceₚ}. Basis 116 is picked by master learner 114 from model redox data 112 yielded by reference bioprocess model 106 (see FIG. 1B). Knowledge of this useful basis 116 and model data 112 enables algorithm 130 to organize measured redox data 124Z in a useful way. Namely, algorithm 130 expresses the portion of measured redox data 124Z that is structured in vector form to be decomposed or expressed in basis 116. This applies to feature vector 112C″ but not to list 112E′*.

A purpose of distributed learning algorithm 130 of learning system 100 (see FIGS. 1A-B) is to determine, discover or learn an optimal composition of measured redox data 132. Optimal redox data 132 are those that should be chosen or included in the set of measured redox data 124Z that is collected under local conditions 202Z from subject 201Z undergoing the bioprocess. In cases where algorithm 130 has already determined optimal redox data 132 and local learner 118 is collecting measured redox data 124Z according to this optimal selection, measured redox data 124Z correspond to optimal measured redox data 132 and are expressed in basis 116.

The establishment of basis 116 by master learner 114 is used in determining optimal measured redox data 132. Expressing the structured portion of redox data, whether from the model (i.e., model redox data 112) or measured (i.e., measured redox data 124) in terms of feature vectors in common basis 116 allows the necessary comparisons and learning to take place. In other words, common basis 116 for the model and measured data permits evaluation in a common context (otherwise, the data may not be commensurate). Thus, a useful comparison between structured model and measured data could not be made for the purposes of machine learning.

In the present exemplary case, learning algorithm 130 deploys basis 116 and then corroborates it by studying the differences between series of measured feature vectors 112CS″ from measured redox data 124Z amongst each other and with model feature vectors 112C′ found in model redox data 112. In other words, learning algorithm 130 deploys learning approaches to evaluate measured feature vectors ĉ and ideal or model feature vectors c. Algorithm 130 can then determine whether measured feature vectors ĉ exhibit behavior expected from bioprocess reference model 106.

The first step in this process relies on proper decomposition of model feature vector 112C′ and measured feature vector 112C″ over the vectors in basis 116. The decomposition can be performed in any suitable manner known to those skilled in the art. If possible, however, the decomposition attempts to maximize independence between the redox indicators. This means that, learning algorithm 130 picks the best basis vectors {ce₁, ce₂, ce₃, . . . , ceₙ} such that the decompositions take on the following form:

112C′=c={c₁,c₂, . . . ,cₙ}=(c₁·ce₁)+(c₂·ce₂) . . . +(c·ceₙ); [Eq. 2A]

112C″=ĉ={ĉ₁,ĉ₂, . . . ,ĉₙ}=(ĉ₁·ce₁)+(ĉ₂·ce₂) . . . +(ĉₙ·ceₙ). [Eq. 2B]

Clearly, the above decomposition is sensitive to deviations in behavior between model and measured redox indicators. It allows algorithm 130 to determine whether the time series of measured feature vectors ĉ(t) agree with expectations set by model feature vectors c(t). This means that algorithm can monitor the unfolding of the bioprocess occurring in subject 201A against the model.

FIG. 2C illustrates learning algorithm 130 comparing a specific measured features vector ĉ with its model counterpart feature vector c. All redox indicators making up the data entries of the feature vectors are compared as shown. If correspondences are not found then the measurement of the particular redox indicator can be dropped. In fact, exactly such an adjustment is shown in FIG. 2C, where only data entries or redox indicators (ĉ₁,ĉ₂,ĉ₄ of measured feature vector 112C″ behaving in predictable ways are retained in optimal feature vector 132C. In other words, measured redox data 124Z part represented by measured feature vector 112C″ is reduced to just the few redox indicators {ĉ₁,ĉ₂,ĉ₄} that are also found to decompose over observable basis 116 established by master learner 114.

Per Eq. 2B, decomposition of measured feature vector 112C″ over the vectors in basis 116 is preferably as follows:

112C″=ĉ={ĉ₁,ĉ₂,ĉ₄}=(ĉ₁·ce₁)+(ĉ₂·ce₂)+(ĉ₄·ce₄).

In other words, in the preferred deployment of learning algorithm 130, measured feature vector 112C″ not only includes the redox indicators that are in the observable basis 116, but each redox indicator is the coefficient associated with one of the basis vectors. Under these conditions the measures of the local bioprocess can effectively focus on just the observable measures, i.e., observable redox indicators in the real vector space spanned by basis 116.

Of course, measured redox data 124Z also contains a contextual part. This part is in the list captured by measured redox data 112E′* in the fifth category. This category may contain data that does not directly pertain to or represent redox indicators {(ĉ₁,ĉ₂,ĉ₄} in observable basis 116. For example, measured redox data 112E′* may contain contextual data or data with as yet unknown relationship to redox indicators {ĉ₁,ĉ₂,ĉ₄}. The measured redox data is also understood to optionally include data about probabilities, statistical relationships and/or any or other information that appears to pertain or may be found through learning by distributed learning algorithm 130 to pertain to one or more redox indicators {ĉ₁,ĉ₂,ĉ₄}.

In some cases, measured redox indicators {ĉ₁,ĉ₂,ĉ₄} contain at least one commonly accepted redox indicator. In other words, in such cases at least one of the measured redox indicators should not be an untested quantity. Particularly useful and established electron balance indicators include indicators consisting of an oxidoreductase, an oxidoreductase co-factor, an electron balance influencer compound, an electron balance influencer composition, a redox-active compound, a pK value, a pH value, a threshold value, a context measure and a soft indicator.

Furthermore, in many cases, the useful redox indicators will optimally be measured on short time scales in comparison to GPR times, as already indicated above. Hence in advantageous embodiments the at least one electron balance indicator is measured with a frequency of at least once every hour, at least once every 30 minutes, at least once every 10 minutes, at least once every 5 minutes, at least once every minute, at least once every 30 seconds, at least once every 10 seconds, at least once every 5 seconds, at least once every second, at least twice every second, at least 5 times every second, at least 10 times every second, at least 20 times every second, at least 50 times every second, at least 100 times every second, or more.

FIG. 2D is a diagram showing the representation of hidden states in a reference learning model 131 used by learning algorithm 130. Hidden states XC1, XC2, . . . , XCj are placed in reference learning model 131 and connect to observable redox indicators in both model and measured feature vectors 112C′, 112C″. They are inaccessible or not measurable parameters that include individual redox states, redox-related parameters or other inaccessible aspects of the bioprocess of interest transpiring in subject 201Z.

For purposes of illustration, the diagram of FIG. 2D expands in the first highly magnified section A to the cell level. Here we see a cell 203 of subject 201Z. Shown in detail are mitochondria 203A and cell nucleus 203B. A second highly magnified section B enlarges a portion of mitochondria 203A to the physical chemistry level. At this level, we find redox couple 104 including redox couple members 104A, 104B and an oxidoreductase or a co-factor 205.

Many aspects of redox status inside mitochondria 203A may not be accessible to measurement. In particular, internal parameters, such as, e.g., internal pH or pH may not be obtained by measurement device 122Z. Thus, internal pH of mitochondria 203A would not qualify as an observable redox indicator for inclusion in feature vector 112C′. However, internal pH of mitochondria 203A clearly influences the redox status in the bioprocess of interest. In fact, the Nernst equation would have to be used to determine just how much the redox potential is affected by internal pH of mitochondria 203A.

In this context, therefore, internal pH of mitochondria 203A would be taken to correspond to a hidden state. Of course, in most cases described herein the hidden state is understood to be the cumulative state over many hundreds, thousands or even larger numbers of reacting entities in the system or sub-system of interest; i.e., many mitochondria 203A. In the present situation, internal pH is represented in reference learning model 131 of distributed learning algorithm 130 by hidden state XC1. Hidden state XC1 is shown to affect measurable redox indicators c₁ and c₂ in accordance with well-known hidden state models, e.g., the Hidden Markov Model.

Redox reactions between redox couple members 104A, 104B aided by oxidoreductase or co-factor 205 at the physical chemistry level, as visualized in highly magnified section B of mitochondria 203A, may likewise be inaccessible to measurement. Therefore, redox reactions between redox couple members 104A, 104B would also be taken to correspond to a hidden state of reference learning model 131. In this case they correspond to hidden state XC2 that stands for the redox potential Eₕ of redox pair 104 in reference model 131 being run by distributed learning algorithm 130. Hidden state XC2 is shown to affect measurable redox indicators c₂ and c₃.

Hidden states XC1, XC2, . . . , XCj are interconnected. Interconnections are associated with transitions and transition probabilities in accordance with standard hidden state models, e.g., the Hidden Markov Model. In FIG. 2D the transitions are indicated with dashed arrows. Such transitions are probabilistic and are part of the bioprocess reference model 106 and more specifically still of reference learning model 131. That is because model 106 is based on curated reference model redox data 108 collected from previous runs and tests of the bioprocess. These include, whenever possible, actual measures of hidden states XC1, XC2, . . . , XCj and transitions between them. Of course, these hidden states are not accessible under local conditions.

The curated model redox data 108 that contains information about transitions between hidden states XC1, XC2, . . . , XCj is preferably further corroborated or validated by model redox data 152 obtained from reference biological entity or live subject 150 undergoing the bioprocess of interest in the lab (see FIG. 1B). In addition, transition probabilities are preferably further tuned during the learning process in accordance with standard rules for computing a transition matrix, as is known to those skilled in the art.

FIG. 2E affords a more detailed look at transition probabilities p_{1,2}, p_{2,1}, p_{3,j}, p_{j,3} between hidden states XC1, XC2, XC3 and XCj. The first subscript on p_{i,j} refers to the initial hidden state before the transition. The second subscript refers to the final hidden state after transition. We use lower case letters p_{i,j} (rather than the traditional upper case) to denote transition probabilities between hidden states XC1, XC2, XC3 and XCj because they are inaccessible. Still, hidden states XC1, XC2, XC3 and XCj directly affect data entries or measured redox indicators {ĉ₁,ĉ₂,ĉ₄} in measured feature vector 112C″. (Note that these same redox indicators have been selected as optimal redox indicators for optimal feature vector 132C by algorithm 130.) A transition matrix p is used by algorithm 130 to keep track of transition probabilities p_{1,2}, p_{2,1}, p_{3,j}, p_{j,3}. Transitions between all hidden states XC1, XC2, . . . , XCj are accounted for by transition matrix p as follows:

$$p = [ \begin{matrix}{p}_{1 , 1} & ⋯ & {p}_{1 , j} \\ ⋮ & ⋱ & ⋮ \\ {p}_{j , 1} & ⋯ & {p}_{j , j}\end{matrix} ] . \qquad [ Eq . 3 ]$$

As illustrated in FIG. 2E, hidden states XC1, XC2 and XC3 are the only ones from which the bioprocess of interest is expected to yield measured redox indicators {ĉ₁,ĉ₂,ĉ₄}. Hidden state XCj is specifically not expected to correspond to a state of the bioprocess that is capable of yielding any locally measurable redox indicator. Still, because of transition probabilities p_{3,j}, p_{j,3} the full transition matrix p has to be used to ensure probability conservation by learning algorithm 130.

Learning algorithm 130 trains or learns on sets of measured redox data 124Z from subject 201Z (see FIG. 2C) and other similar subjects. In accordance with standard learning methods, algorithm 130 iteratively reviews relevant transition probabilities p_{1,2}, p_{2,1}, p_{3,j}, p_{j,3} originally obtained from reference learning model 131 to adjust them as needed. Preferably, measured redox data 112E′* contains measured list entries [ê₁,ê₂, . . . ,ê_{y}] of both redox indicator candidates and unstructured data to aid in this process. Furthermore, the transition matrix and the condition for conservation of total probability are used by algorithm 130 to ensure that any adjustments to transition matrix p obey the rule of conservation of probability.

In addition to transitions between hidden states XC1, XC2, XC3, . . . , XCj reference learning model 131 deployed by learning algorithm 130 assigns probabilities to measurement outcomes. These are measurement probabilities leading to observable redox indicators. They are hence denoted by the traditional upper case P_{i,j}. Specifically, if the bioprocess of interest is in hidden state XC1 it has a measurement probability P_{xc1,c1} of yielding observable redox indicator c₁. From the same hidden state XC1, it has a measurement probability P_{xc1,c2} of yielding observable redox indicator c₂.

Outcomes or measurement transition probabilities from hidden states are part of the bioprocess reference model 106 and its reference learning model 131. Model 106 is based on curated reference model redox data 108 collected from previous runs and tests of the bioprocess that includes measurement probabilities. As in the case of transition probabilities, the curated model redox data 108 that contains information about measurement transition probabilities between hidden states XC1, XC2, XC3 and measured redox indicators {ĉ₁,ĉ₂,ĉ₄} is preferably further corroborated or validated by model redox data 152 obtained from reference biological entity or live subject 150 undergoing the bioprocess of interest in the lab (see FIG. 1B). Measurement probabilities are preferably further tuned during the learning process in accordance with standard rules known to those skilled in the art.

In the case shown in FIG. 2E, learning algorithm 130 obtains relevant measurement probabilities P_{xc1,c1}, P_{xc1,c2}, P_{xc2,c2}, P_{xc2,c3}, P_{xc3,c4} from reference learning model 131 that is part of model 106 and tunes them during learning. Note that conservation of probability can be used in order to properly account for all outcomes. This is analogous to tracking transition probabilities between hidden states. Specifically, measurement probability P_{xc2,c3} is still present, but measured redox indicators {ĉ₁,ĉ₂,ĉ₄} in measured feature vector 112C″ do not include observable redox indicator c₃. Thus, the corresponding measurement probability becomes hidden. For this reason, measurement probability P_{xc2,c3} and measurable but not actually measured redox indicator {ĉ₃} are indicated in hatched boxes.

Preferably, list of model redox data 112E* contains information about candidates for measurable redox indicators under local conditions and in changing contexts. Specifically, list 112E* preferably indicates that measurements from hidden state XC2 will not be fully reflected when redox indicator c₃ is dropped from optimal feature vector 132C. In fact, reference bioprocess model 106 preferably provides distributed learning algorithm 130 with a preliminary set of expected hidden states, transition probabilities and measurement probabilities for reference learning model 131 in list 112E. Thus, algorithm 130 running on master learner 114 does not have to start learning these parameters without guidance. Instead, algorithm 130 tunes these parameters based on learning from measured redox data 124Z. When a major deviation or correction is discovered by algorithm 130, then it can send this data to reference bioprocess model 106 in update 134, as shown in FIGS. 1A-B. In other words, master learner 114 may initialize local learner 118 with an initial set of weights or initial conditions from reference bioprocess model 106 to increase the chance that local learner 118 will be able to converge more rapidly given the computational resources.

Information captured by measured redox data 112E′* in the fifth category can also contain data that does not directly pertain to redox indicators {ĉ₁,ĉ₂,ĉ₄} in observable basis 116. For example, measured redox data 112E′* may contain contextual data or data with as yet unknown relationships to redox indicators {ĉ₁,ĉ₂,ĉ₄}. Such relationship may then be found through learning by distributed learning algorithm 130.

As also indicated in FIG. 2E, learning algorithm 130 can further condition observable redox indicators {c₁, c₂, c₄} by assigning a weighting or a confidence level to one or more of them using a conditioning module 210. Such assignment allows for local tuning beyond adjusting measurement probabilities or transition probabilities. For example, confidence levels and weightings can represent relative confidence in the local measurement process, or can be used to factor in the availability, practicality or cost of certain local measurement parameters. Furthermore, since the reactions of interest concern electron balance, learning algorithm 130 can focus on just observable redox indicators that are measured on time scales shorter than Gene-Protein-Reaction (GPR) time.

Upon learning from both reference bioprocess model 106 and the local bioprocess learning algorithm 130 can keep changing or adjusting redox indicators {c, c₂, c₄} decomposed over observable basis 116. Of course, any material learned adjustment in observable basis 116 of redox indicators should be communicated to master learner 114. Also, reference bioprocess model 106 can be configured to receive a reference model adjustment from learning algorithm 130 based on what it has learned. Reference model adjustment 134 can involve an alteration in model redox data 112, an alteration in the model conditions or an alteration in the hidden states postulated to exist in reference learning model 131.

Learning system 100 can employ many general methods that extend beyond working from just reference learning model 131 initially used by learning algorithm 130. In other words, learning algorithm 130 that engages in learning the optimal composition of measured redox data 132 or of observable redox indicators {c₁, c₂, c₄}, say by choosing them from a general set of redox indicators need not be implemented within any one particular learning paradigm. In fact, learning system 100 can employ one or more learning methods. Some particularly useful methods in the embodiments of the present invention include Artificial Intelligence (AI) methods, Hidden Markov methods and Deep Learning (multi-layered neural network) methods. Any of these methods can be implemented in the recursive feedback structure presented by learning system 100 of the invention.

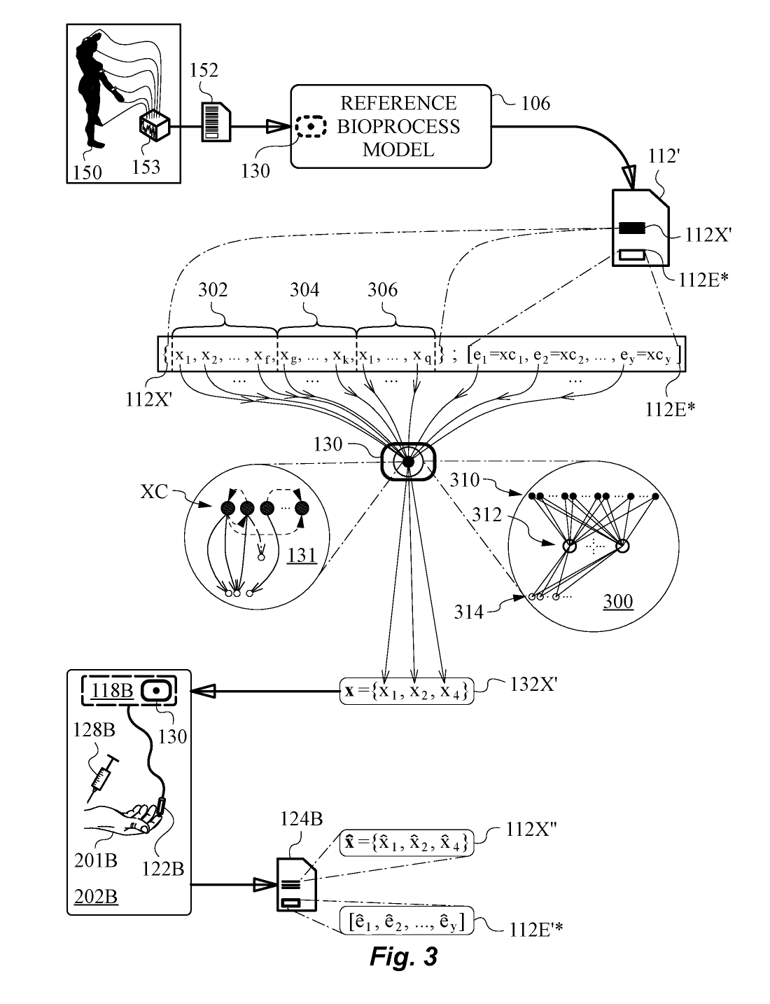

FIG. 3 is a diagram illustrating in more detail a specific learning method. This learning method is embodied by a neural network learning model 300 deployed by learning algorithm 130. In this embodiment, reference bioprocess model 106 is constructed from model redox data 152 obtained from reference biological entity 150 as shown in FIG. 1B. As in the previous embodiment, distributed learning algorithm 130 starts from reference learning model 131.

In this example reference bioprocess model 106 collapses the four redox categories into a single joint model feature vector 112X′. It also provides model redox data 112E* enumerating possible alternative candidate redox indicators xc₁, xc₂, . . . , xc_{y}. These candidates could be used in joint model feature vector 112X′. Thus, model redox data 112′ contains just joint model feature vector 112X′ and list 112E*.

The exploded view of joint model feature vector 112X′ at a specific time (not expressly indicated in the present drawing) shows a further subdivision in the vector's data entries. Specifically, as shown, model redox indicators x₁, x₂, . . . , x_{f} belong to a first panel 302 corresponding to the second redox principle or category (the of redox electron transfers to adjust protein structure through kinetically controlled redox switches, a.k.a. as S-switches or Sulphur switches). Model redox indicators x_{g}, . . . , xₖ belong to a second panel 304 of redox indicators that are likely in the first redox category or in the fourth redox category. Model redox indicators x₁, . . . , x_{q} are redox indicators that cannot be clearly identified with any category. These unassignable redox indicators are put in a third panel 306.

In the present example, neural network learning model 300 receives joint model feature vector 112X′ at its inputs 310. Hidden layer 312 of model 300 deploys neural learning to determine a series of outputs 314 that best satisfy a learning criterion. In the present case, the learning criterion is the selection of optimal composition of measured redox data 132. More specifically, the optimal composition of redox indicators to be used in joint feature vector 112X′—i.e., optimal joint feature vector 132X′

Preferably, model 300 runs alongside reference learning model 131 based on hidden states XC that are merely inaccessible, but physically real, as described above. At the onset, outputs of reference learning model 131 suggest that optimal joint feature vector 132X′ to be measured in measured redox data 124B collected from subject 201B under local conditions 202B should be {x₁, x₂, x₄}. This is indeed measured joint feature vector 112X″.

Over time, however, deep learning model 300 is expected to diverge from reference learning model 131 in its suggestion of optimal joint feature vector 132X′. This is expected because deep-learning model 300 which will introduce by its very nature non-physical hidden layers and states without any direct correspondence to hidden states XC of reference learning model 131. As long as such states have a material effect on redox status they should be postulated in learning model 300 as a part of the deep-learning process. Distributed learning algorithm 130 should start using the recommendation of learning model 300 as soon as the latter starts performing better than reference learning model 131 on which distributed learning algorithm 130 started.

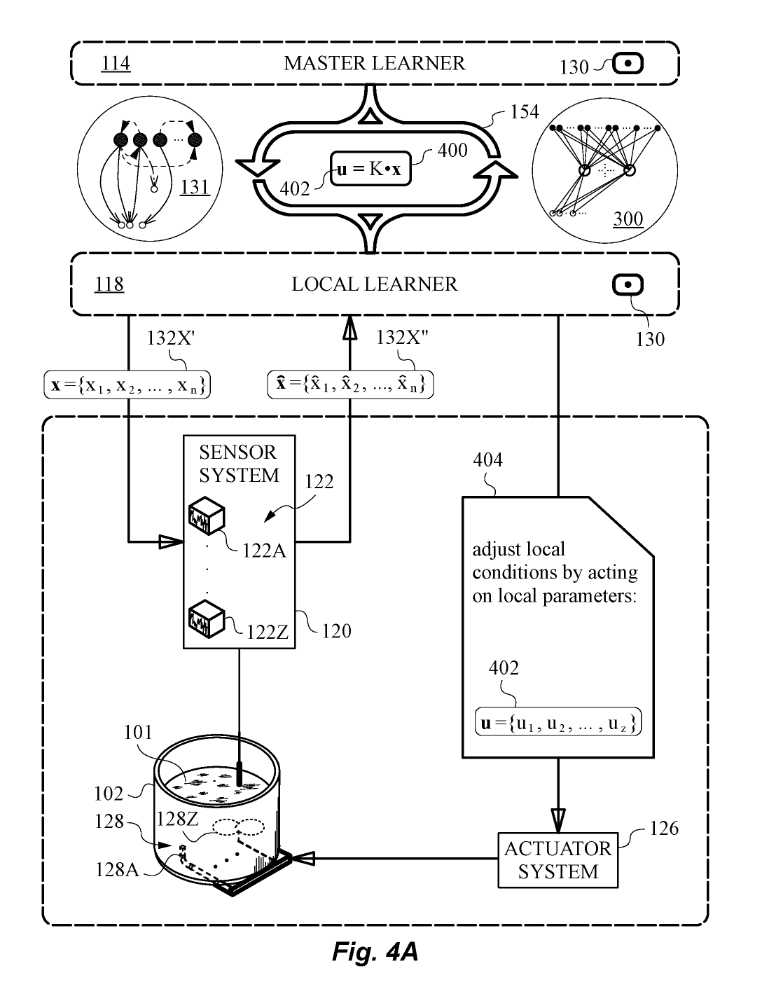

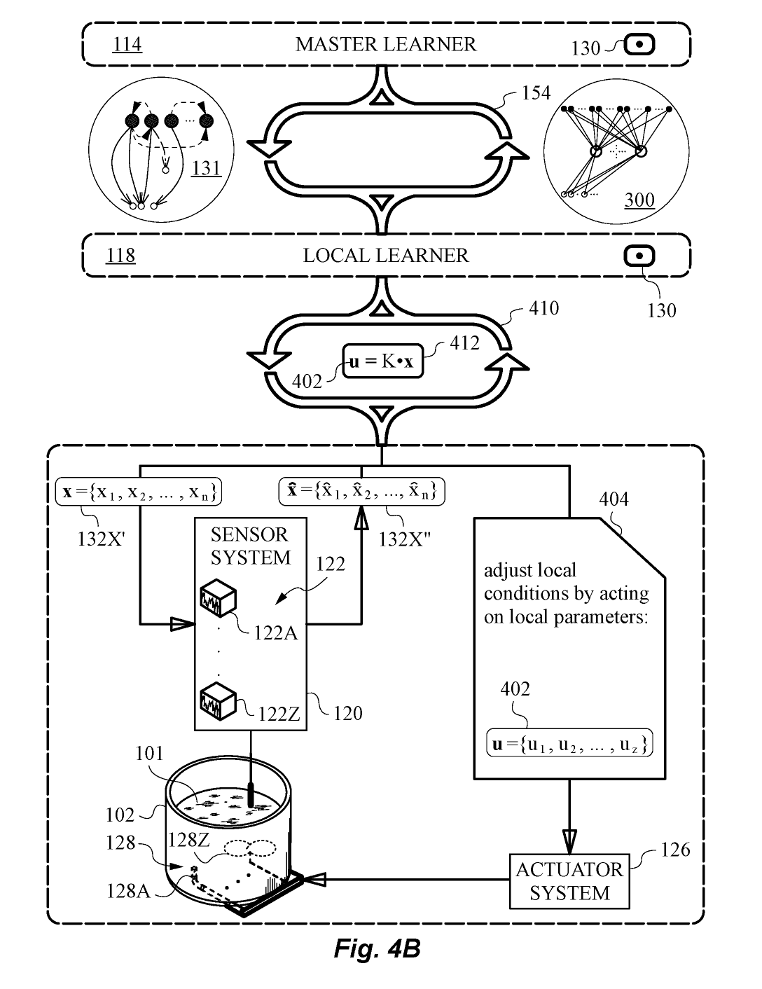

FIG. 4A shows an embodiment in which learning algorithm 130 can learn how to adjust local conditions by making adjustments to local control parameters. For this reason, the at least one local entity that is undergoing the bioprocess is preferably configured to receive a local control parameter adjustment from the learning algorithm via whatever local affordances are available. For exemplary purposes, we review the adjustment of local conditions for an embodiment in which the bioprocess of interest is transpiring in bioreactor 102 of learning system 100 as shown in FIG. 1A. Only the relevant parts of system 100 from FIG. 1A are shown in FIG. 4A for reasons of clarity.

FIG. 4A illustrates aster learner 114 and local learner 118 cooperatively learning about the bioprocess of interest in bioreactor 102 with the aid of distributed learning algorithm 130. Primary feedback loop 154 is sharing the results of tuning and adjustments to reference learning model 131 and the learning achieved by deep learning model 300 between learners 114, 118.

The results of learning by learning algorithm 130 produce optimal feature vector 132′. More precisely, distributed learning algorithm 130 started with reference learning model 131 and its suggesting for redox indicators given conditions in bioreactor 102 and contextual information. Reference learning model 131 was then run alongside deep learning model 300 to corroborate the choice of redox indicators for optimal feature vector 132′. The distributed learning yielded optimal feature vector 132′ after a number of iterations (potentially in corroboration with other instances of the bioprocess of interest being run at other locations under correspondent local conditions). It is this optimal feature vector 132′ that local learner 118 requests to be measured by local sensor system 120.

Optimal feature vector 132′ contains a number n of redox indicators in all four redox principles. The optimal redox indicators are thus contained in the first four redox categories 112A, 112B, 112C and 112D (see, e.g., FIG. 2A and the corresponding teachings). However, because of local inability to distinguish between redox principles, optimal feature vector 132′ is a joint optimal feature vector 132X′. In vector 132X′ all redox categories have been collapsed or combined into a single vector. The number n of entries of optimal feature vector 132X′ are expressed in joint basis 116 as {x₁,x₂, . . . ,xₙ} according to the notation convention introduced above. Following the same convention, measured optimal feature vector 132X″ expressed in basis 116 is {\hat{x}₁,\hat{x}₂ . . . ,\hat{x}ₙ}.

Local learner 118 requests that sensor system 120 use appropriate measuring devices 122 to collect from bioreactor 120 redox indicators in optimal feature vector 132X′. Correspondingly, sensor system 120 deploys specific measurement devices 122A-Z to collect a time series of optimal measured feature vectors 132XS″ with the desired redox indicators. Only one optimal measured feature vector 132X″ of the series is shown in the diagram of FIG. 4A for reasons of clarity. Local conditions inside bioreactor 102 can be adjusted with the aid of actuator system 126. Actuator system 126 has at its disposal a number of specific actuators 128 to act on local control parameters in bioreactor 102. In the present embodiment, the adjustments to local control parameters are issued in conjunction with the learning achieved by distributed learning algorithm 130. Since algorithm 130 is distributed, adjustments can be computed and issued from master learner 114 or local learner 118.

When the communication link between learners 114, 118 has a large bandwidth and is reliable, it is advantageous to provide primary feedback loop 154 with a primary feedback mechanism 400. In FIG. 4A primary feedback mechanism 400 is shown to compute an adjustment vector 402 expressed here by u (bold face denotes a vector quantity). Primary feedback mechanism 400 uses its knowledge of the bioprocess of interest and of optimal feature vector 132′, also expressed here as vector x.

Adjustment vector u is arrived by applying matrix K to optimal feature vector x (and/or measured optimal feature vector 2). Derivation of the K matrix is a standard problem in control theory. In the present case, the computation of K should reflect local conditions in bioreactor 102, context and local constraints and measurement capabilities, including the various sources of measurement noise. Persons skilled in the art of control theory and feedback will recognize various approaches for computing the most effective K matrix.

Primary feedback mechanism 400 is configured to issue a local conditions adjustment 404 that will include any general operating instructions (e.g., to the operator of bioreactor 102) as well as specific adjustments. The specific adjustments correspond to entries in adjustment vector 402. They are part of file of local conditions adjustment 404 sent to actuator system 126. In the present case, a number r of control parameters u₁, u₂, . . . , uᵣ make up adjustment vector u sent to actuator system 126. Advantageously, control parameters u₁, u₂, . . . , uᵣ can be adjusted by actions that can be performed by specific actuators 128A-Z (or combinations of their actions) deployed by actuator system 126.

Many if not most control parameters u₁, u₂, . . . , uᵣ will be redox indicators or redox influencers. These can be selected from the same group of candidates as those for feature vectors 112A-D. However, the best candidates for this purpose are redox indicators that can be acted upon directly by actuator system 126. In other words, control parameters should correspond to redox indicators that can be affected in known ways by any one actuator 128 or by any combination of specific actuators 128A-Z. Thus, control parameters u₁, u₂, . . . , uᵣ can include a redox active compound or an electron balance influencer, or still other inputs that can act upon the bioprocess transpiring in local bioreactor 102.

FIG. 4B illustrates an implementation of feedback control to provide local conditions adjustment 404 when communications between local and maters learners 118, 114 are not robust. Not robust can mean low bandwidth, noisy and/or subject to frequent or unacceptable interruptions. Under such conditions it is preferable to rely on a secondary feedback loop 410 established between local learner 118 and the biological entity of interest. In this example, the biological entity of interest is again biomass 101 in bioreactor 102, as also shown in FIG. 4A. It is noted, that biological entities of interest can be organisms including live subjects 201.

Secondary feedback loop 410 is set up between local learner 118 and local resources that run sensor system 120 and actuator system 126. Thus, feedback loop 410 channels the local connections that were previously sent to local learner 118 (see FIG. 4A). These connections include the ones for transmitting optimal feature vector 132X′ and measured optimal feature vector 132X″ to and from sensor system 120.

Secondary feedback loop 410 has a local feedback mechanism 412. In operational respects, local feedback mechanism 412 performs the work of primary feedback mechanism 400 (see FIG. 4A). Thus, local feedback mechanism 400 determines the K matrix and also adjustment vector 402 also represented by u. Local feedback mechanism 400 also issues local conditions adjustment 404 that will include any general operating instructions (e.g., to the operator of bioreactor 102) as well as specific adjustments. As before, specific adjustments correspond to entries in adjustment vector 402. They are part of file of local conditions adjustment 404 sent to actuator system 126.

In the embodiments of FIGS. 4A-B and in general, local conditions adjustment can involve an alteration in the optimal composition of measured redox data, redox candidate data, contextual data and any additional data related to the subject. In other words, the adjustments can extend beyond those that can be expressed in adjustment vector 402 and applied directly. Of those that can be acted on by actuator system 126 with its specific actuators 128, the most commonly are parameters affecting: off-gas, air, O₂, CO₂, pressure, viscosity, stirrer speed, temperature, pO₂, pH, photometrics, calorespirometric measures and other biomeasurables. Of course, there may be cases in which control of the local bioprocess is impossible or impractical. This could occur in rapidly transpiring reactions or reactions that go to completion without allowing for meaningful intervention. No local feedback mechanism may be present in such embodiments.

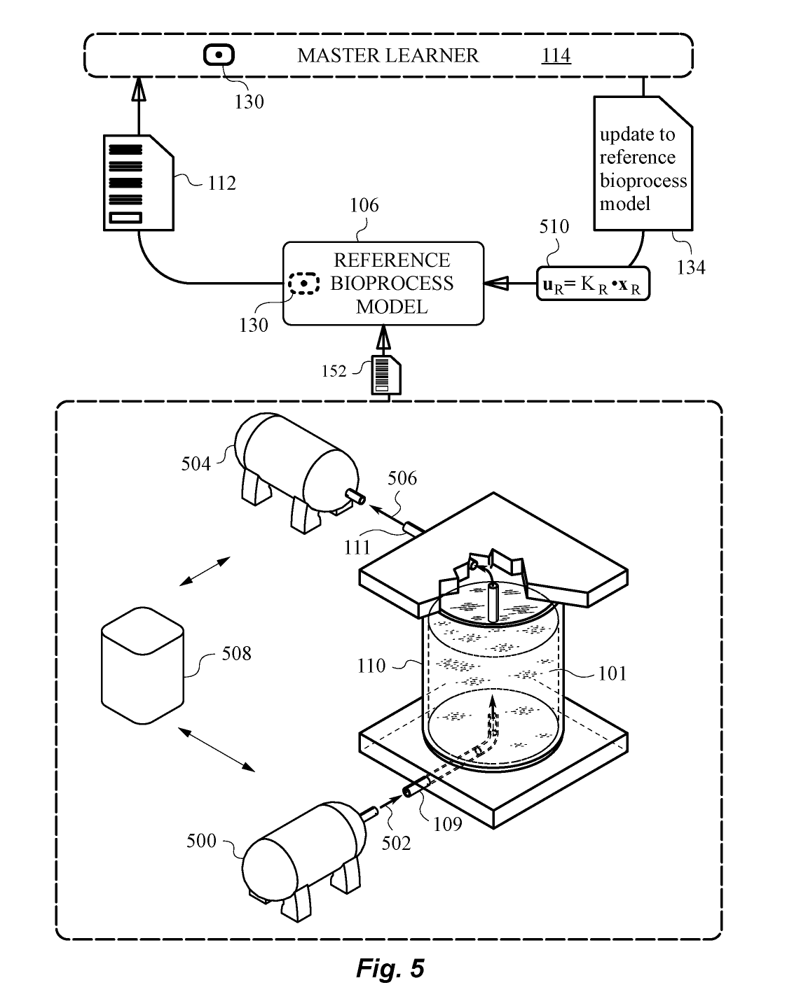

FIG. 5 is a diagram illustrating a reference bioprocess performed in a reference bioreactor with adjustments to reference control parameters. This is done when, as a result of the learning performed by learning system 100, it becomes necessary to change the operation of the reference biological entity undergoing the bioprocess on which the model is based. As an example, we take reference bioprocess model 106 derived from model redox data 152 collected from reference bioreactor 110 (see FIG. 1A).

Reference bioprocess is transpiring in biomass 101 within reference bioreactor 110. An input 109 to reference bioreactor 110 is provided for adjusting or altering reference bioprocess occurring inside it. Input 109 is to be understood generally as any mechanism, actuator, inlet or other type of mechanical or non-mechanical apparatus capable of acting on the bioprocess. Actuator systems or mechanisms 500 interface with input 109. Mechanisms 500 are capable of making input adjustments 502 to the conditions in reference bioreactor 110 as a result of learning that occurs during construction of reference bioprocess model 106.

Likewise, an output 111 is provided for drawing outputs or samples from the bioprocess unfolding within biomass 101 inside reference bioreactor 110. Sensing or measuring apparatus 504 interface with output 111. Measuring apparatus 504 is to be understood generally as any apparatus or device capable of drawing, collecting, inferring, sensing and measuring outputs 506 of the bioprocess. Measuring apparatus 504 can use outputs 506 in any direct in-line measures such as: off-gas, air, O₂, CO₂, pressure, viscosity, stirrer speed, temperature, pO₂, pH, photometrics, calorespirometric measures and other biomeasurables. Measuring apparatus 504 can also obtain indirect in-line measures by techniques such as: near-infrared spectroscopy, dielectric spectroscopy, fluorescence spectroscopy, Fourier-transform infrared spectroscopy, Raman spectroscopy. The sampling methods and measures that can be used include: high performance liquid chromatography, enzyme-linked immunosorbent assay, gas chromatography, electrophoresis microscopy, mass spectroscopy, proton transfer reaction MS, MALDI-TOF MS, nuclear magnetic resonance, flow injection analysis. In addition, measuring apparatus 504 can apply data or model-driven analysis to derive measures such as: levels or quantities of active biomass 101, glucose, lactate, amino acids, enzymes, antibodies, organic acids, vitamins, recombinant proteins, volatile organic compounds.

Actuator mechanisms 500 and measuring apparatus 504 are connected to a central reference coordinator unit 508. Unit 508 coordinates the regular operation of reference bioprocess and production of model redox data 152. In addition, reference coordinator unit 508 receives updates 134 sent from master learner 114 to reference bioprocess model 106 that is based on model redox data 152. In fact, central reference coordinator unit 508 can be in charge of running reference bioprocess model 106 on its own resources in some embodiments. In such embodiments, the inputs or outputs of reference bioprocess model 106 discussed above, will refer to inputs and outputs of the computer or computer system(s) of unit 508. Clearly, a module of distributed learning algorithm 130 will then run on unit 508 as well.

In order for unit 508 to implement the learning that algorithm 130 derived from the one or more local reactors 102 (see FIG. 1A) that perform the same bioprocess a reference feedback mechanism 510 is provided between master learner 114 and reference bioprocess model 106. In the event model 106 is running on unit 508, reference feedback mechanisms 510 is established between master learner 114 and unit 508. The fact that mechanism 510 refers to the reference bioprocess and its model is expressed by the subscripts “R” on the vectors and the matrix.

Given that mechanism 510 executes directly on reference biological entity, here biomass 101, the feedback is actually provided between master learner 114 and the reference biological entity. For the purposes of applying the feedback, unit 508 can simply use all of the already available affordances. Specifically, unit 508 uses actuator mechanisms 500 for making input adjustments 502.

In embodiments where there is no physical reference biological entity that provides model redox data 152, i.e., there is neither a reference bioreactor 110 or a reference biological entity or live organism including such as a human subject then it may become necessary to simply tune or adjust reference bioprocess model 106 on curated data 108 alone (see FIGS. 1A-B).

In some embodiments, the bioprocess will occur without supervision, while in other cases the bioprocess can be a tightly supervised process. In any case, the bioprocess in the local biological entity will typically occur under much less controlled conditions than those of the reference biological entity that was used in the reference bioprocess model.

In some embodiments, the elements of the learning system are directly coupled to each other as part of an integrated system. In other embodiments, the system elements may be in separate physical systems and coupled by one or more application program interfaces. In still other embodiments, the measurement systems are indirectly connected to the learning system by exporting data in formats that can be imported or scanned into the database accessed by the local or master learner. In still other embodiments, the system is directly connected to a control mechanism, while in other embodiments the control may be a recommendation to another system or operator, or may not be present at all. Also, there are embodiments in which the control mechanism provides an instruction to a third party system for formulation of a nutritional, supplement, vitamin, medication or combination.

The chemical reaction networks that underlie cellular processes are complex systems built upon non-deterministic and ultimately even quantum mechanical interactions that have an inherent level of random fluctuation or noise. This creates a level of unpredictable variation that may limit the contexts in which any deterministic or classical learning model may apply. This inherent noise indeed may be the basis for the evolution and diversity of life in the first place. While it is tempting to think that if all the parameters of a biological system were known, measurable, and tunable, that one could perfectly control health and disease in biological systems, this is unlikely. Consequently, this invention provides an alternative approach that assumes imperfect measurement, hidden states, and inherent limits to observability and controllability of the state of any biological entity under consideration. Despite these inherent limits, biological entities and larger biological systems strive for homeostasis, or stability. In such a stable state of “health” where the reduction and oxidation systems of energy production are in balance without causing damage over sustained periods of time. Living systems also can slip into states of “disease” when the reduction system begins to fail and the oxidation systems of energy production cumulate damage. Such accumulation increases the chance that the entire biological entity or system eventually enters a cascading failure resulting in death.

In other words, a healthy state of a biological entity or system is one in which it and its internal regulatory system can balance the disturbances and pressures of the internal and external environment. This healthy state is not a singular point within the space of possibilities but rather an attraction basin in which the system as a whole is stable despite the inherent random fluctuation or noise in a large number of component parts. A complex biological entity or system can be maintained over time in such a quasi-potential basin despite the inherent noise in its component parts and within a variety of environmental contexts and disturbances. This is largely because of its internal regulatory processes that continuously tune a large number of parameters. Such a complex system is stable when the quasi-potential basin is deep and the walls are high in comparison with the inherent noise. Under these conditions the system can continuously make small adjustments that keep moving the state toward the basin. A working reduction system that counteracts the damaging effects of oxidation in a metabolic process despite a wide range of environmental variation and stress is a regulatory process aiming to keep the biological entity or system in a stable state.

As life evolved over 4 billion years, nature's internal regulatory systems have been highly adapted after generations of natural selection to take advantage of any optimizations or efficiencies afforded by physics and chemistry. This includes the ability of quantum systems to take advantage of non-classical features such as coherence and quantum correlations (e.g., entanglement) to optimize processes and store information. As such, the evolved biological system has available to it a much larger set of tunable parameters within a broader set of paradigms than those designed for modern medicine and other life sciences. The regulatory approaches proposed by modern biotechnology are primarily attempts to fix or tune single inputs or very simple sets of tunable inputs to a classically described biological entity or system. These approaches have been successful in some contexts where a single or a very small set of tunable parameters can restore a balance or compensate for an imbalance in the biological entity or system.

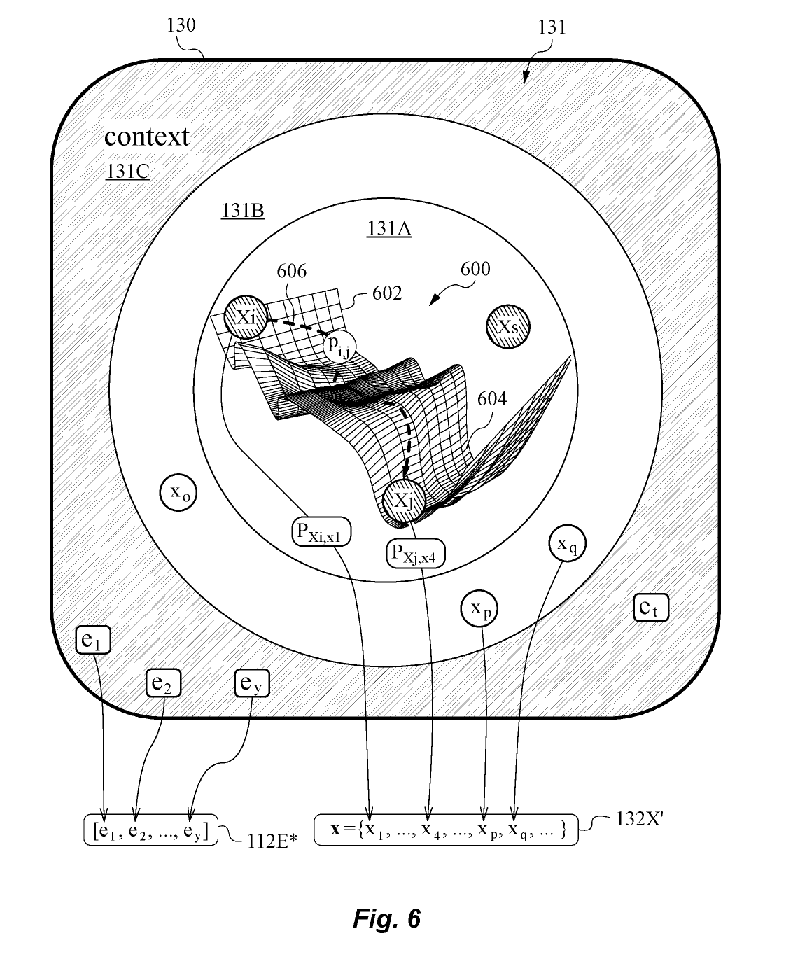

We turn to the diagram of FIG. 6 in light of the above to examine one of the reasons for explicit introduction of hidden states. Only three hidden states Xᵢ, Xⱼ and Xₛ (where capital letters designate hidden states) for reasons of clarity. In the example of FIG. 6 distributed learning algorithm 130 and preliminary learning model 131 are given an abstract representation different than a graph structure (e.g., FIG. 2E).

In FIG. 6 preliminary learning model 131 is broken up into three domains. At the very center is a hidden domain 131A delimited by the inner circle and containing hidden states Xᵢ, Xⱼ and Xₛ. Hidden domain 131A uses a representational space 600 within which is embedded a multi-well quasi-potential 602. Effectively, quasi-potential 602 is a landscape (sometimes also referred to as fitness landscape by those skilled in the art) that states Xᵢ, Xⱼ can be considered to inhabit. When using other classical models, representational space 600 may introduce a phase space spanned by certain conjugate variables or still another useful abstraction known in the art. When using quantum models, representational space 600 may introduce Hilbert space or even Fock space.

The topology of quasi-potential 602 dictates possible evolution between states (transitions or dynamics). It also graphically shows where meta-stable and stable states (wells) are to be found. In the present example, a transition between hidden state Xᵢ and hidden state Xⱼ may occur with a transition probability p_{i,j} (recall that lower case denotes transition probabilities between hidden states, as before). Clearly, given exemplary landscape 602, hidden state Xⱼ is quite stable. That is because it is in a deep potential well 604 with high potential barriers or walls. Hidden state Xᵢ is only meta-stable because it is not in a deep well.

Perturbations, inherent noise or even intended actions (e.g., introduced by actuator system 126) may aid the transition from hidden state Xᵢ to hidden state Xⱼ. The response to the unintended or intended action is indicated by dashed arrow 606. Arrow 606 illustrates the path in abstract representational space 600 along which the state transition Xᵢ to Xⱼ takes place.

Of course, appropriate actions can also change landscape 602 itself. As will be appreciated by those skilled in the art, such modifications to quasi-potential 602 should be accounted for by an adjustment or tuning of transition probabilities in transition matrix p (see Eq. 3). In the present case, it is especially important to adjust transition probability p_{i,j}.

A second non-hidden and measurable domain 131B of learning model 131 resides between inner hidden domain 131A and a third conditional or context domain 131C. Measurable domain 131B contains states indicated by lower case letters. In the present case, three such measurable states are shown, namely xₒ, xₚ and x_{q}. These states correspond to quantities that are directly measurable both in the lab and under local conditions (in the field). They are typically not associated with hidden aspects or transition probabilities that need to be tracked. Hence, they are not placed in a representational space. Other than being subject to well-known measurement errors, noise etc., states xₒ, xₚ and x_{q} inhabiting measurable domain 131B are directly measurable. Thus, there is no measurement probability associated with them. This is unlike hidden states Xᵢ, Xⱼ and Xₛ inhabiting hidden domain 131A. These, even during measurement, still exhibit a probabilistic aspect that translates into their associated measurement probabilities P_{Xi,x1}, P_{Xj,x4}, P_{Xs,xz} (see FIG. 2E and related description).

Redox indicators or features that correspond to states in either hidden or measurable domains 131A, 131B may belong to redox indicators in any one of the first four redox categories 112A-D. In fact, the careful reader will have noticed that by adopting the joint feature variable names X and x, we have collapsed the first four redox categories 112A-D into one joint category 112X and are using the joint feature vector representation.

Conditional or context domain 131C contains all other conditional redox data in the fifth redox category 112E. Of course, this data can contain candidates for either hidden or measurable states X and x to be placed into hidden or measurable domains 131A, 131B of preliminary learning model 131. In addition, it contains purely contextual data, e.g., the weather. In the present example four specific data entries e₁, e₂, eₜ and e_{y} are shown.

As shown in FIG. 6, preliminary learning model 131 already contains a preliminary contingency list 112E* and preliminary joint feature vector 112X′. These may be selected in reference bioprocess model 106 given the biological entity under study, the bioprocess of interest and the local conditions. Alternatively, this may already be a tuned learning model 131 prepared by distributed learning algorithm 130 after a few iterations of learning between master learner 114 and local learner 118.

In fact, as shown, hidden states Xᵢ, Xⱼ as well as measurable states xₚ, x_{q} corresponding to directly accessible redox indicators are selected from preliminary joint feature vector 112X′ for optimal joint feature vector 132X′. Hidden state X, and measurable state xₒ are not included in optimal joint feature vector 132X′. Also, states or data entries e₁, e₂, and e_{y} are selected for contingency list 112E*. State or data entry eₜ is not chosen. These choices are made given the local conditions and, possibly, preliminary knowledge of context under location conditions.

We can now see some of the reasons for the explicit introduction of hidden states and transitions between them into learning system 100 and the initial or preliminary learning model 131. Postulating hidden states, some of which are inaccessible in principle, provides us with an inherent ability to deal with unknown features. Specifically, the present invention can ascribe to them states and transitions that are hidden and not part of the observable basis of redox indicators 116. Thus, the invention teaches a way to expand the subset of parameters available to model the status of a hidden compartment. This also permits to introduce additional opportunities for tuning parameters or providing related control inputs, e.g., in the form of adjustment vectors. Using further control theory approaches, the inputs or adjustment vectors may aim to maintain or restore balance in the biological entity under the local conditions and within the context. The hidden states approach also sets up a framework in which non-classical features can be explored. Specifically, hidden states may be placed into a classical or even a non-classical state in representation space 600, such as a phase space or Hilbert space.

In terms of measurable redox indicators, in either structured or unstructured form (e.g., feature vectors 112A-D, joint feature vector 112X, or contingency list 112E*) they should include concentrations of compounds from a network of orphan enzymes and small molecules capable of encoding electrons to transfer information rapidly between proteins. This system is comprised of unique enzymes called oxidoreductases, already mentioned above, and unique small molecule redox signaling molecules. The dimensions of this network in biology may be 2,000 enzymes, including 584 human oxidoreductase enzymes, and over 10,000 redox small molecules. The preliminary learning model may initially focus on the subset of this matrix that is common to all biological systems and regulates energy generation. More specifically, the measured redox data includes Flavin-containing oxidoreductase quinones (believed to be critical and common to metabolic control and members of the network with biological functions and importance which has not yet been established).

There are a variety of measurements that could comprise an observable basis of redox indicators 116 for determining the redox status of the bioprocess or other hidden states of the biological entity. There are also variety of tunable inputs with the potential to balance or control the biological entity or complex living system. To account for these in reference bioprocess model 106 a measurement system such as a high-resolution mass spectrometer can be used in a controlled laboratory environment. There, specific enzymes and cofactors from the above-mentioned matrix of possibilities can be upregulated, downregulated or inhibited in a range of cell cultures from a reference biological entity or reference subject 150. These actions can be performed under a range of environmental disturbances or insults, with and without providing reference entity 150 any of a range of rescue compounds, and observed over a range of time slices. Examples of such cell cultures that may be used in the bioprocess reference model 106 can be found in Table 2A. Examples of stressors or insults that can be used in the bioprocess reference model can be found in Table 2B. The measurement time slices to observe the network of reactions following a disturbance or insult in the laboratory can have a frequency of at least once every hour, at least once every 30 minutes, at least once every 10 minutes, at least once every 5 minutes, at least once every minute, at least once every 30 seconds, at least once every 10 seconds, at least once every 5 seconds, at least once every second, at least twice every second, at least 5 times every second, at least 10 times every second, at least 20 times every second, at least 50 times every second, at least 100 times every second, or more.

TABLE 2A

| Cell Line | Description |
|---|---|
| SH-SY5Y | Human neuroblastoma |
| Hep G2 | Human Caucasian hepatocyte carcinoma |
| 293 (also known as | Human Embryo Kidney |
| HEK 293) | |
| RAW 264.7 | Mouse monocyte macrophage |
| HeLa | Human cervix epitheloid carcinoma |
| MRC-5 (PD 19) | Human foetal lung |
| A2780 | Human ovarian carcinoma |
| CACO-2 | Human Caucasian colon adenocarcinoma |
| THP 1 | Human monocytic leukaemia |
| A549 | Human Caucasian lung carcinoma |
| MRC-5 (PD 30) | Human foetal lung |
| MCF7 | Human Caucasian breast adenocarcinoma |
| SNL 76/7 | Mouse SIM strain embryonic fibroblast |
| C2C12 | Mouse C3H muscle myoblast |
| Jurkat E6.1 | Human leukaemic T cell lymphoblast |
| U937 | Human Caucasian histiocytic lymphoma |
| L929 | Mouse C3H/An connective tissue |
| 3T3 L1 | Mouse Embryo |
| HL60 | Human Caucasian promyelocytic leukaemia |
| PC-12 | Rat adrenal phaeochromocytoma |
| HT29 | Human Caucasian colon adenocarcinoma |
| OE33 | Human Caucasian oesophageal carcinoma |
| OE19 | Human Caucasian oesophageal carcinoma |
| NIH 3T3 | Mouse Swiss NIH embryo |
| MDA-MB-231 | Human Caucasian breast adenocarcinoma |
| K562 | Human Caucasian chronic myelogenous |
| | leukaemia |
| U-87 MG | Human glioblastoma astrocytoma |
| MRC-5 (PD 25) | Human foetal lung |
| A2780cis | Human ovarian carcinoma |
| B9 | Mouse B cell hybridoma |
| CHO-K1 | Hamster Chinese ovary |
| MDCK | Canine Cocker Spaniel kidney |
| 1321N1 | Human brain astrocytoma |
| A431 | Human squamous carcinoma |
| ATDC5 | Mouse 129 teratocarcinoma AT805 derived |
| RCC4 PLUS VECTOR | Renal cell carcinoma cell line RCC4 stably |
| ALONE | transfected with an empty expression |
| | vector, pcDNA3, conferring neomycin |
| | resistance. |
| HUVEC (S200-05n) | Human Pre-screened Umbilical Vein |
| | Endothelial Cells (HUVEC); neonatal |
| Vero | Monkey African Green kidney |
| RCC4 PLUS VHL | Renal cell carcinoma cell line RCC4 stably |
| | transfected with pcDNA3-VHL |
| Fao | Rat hepatoma |
| J774A.1 | Mouse BALB/c monocyte macrophage |
| MC3T3-E1 | Mouse C57BL/6 calvaria |
| J774.2 | Mouse BALB/c monocyte macrophage |
| PNT1A | Human post pubertal prostate normal, |
| | immortalised with SV40 |
| U-2 OS | Human Osteosarcoma |
| HCT 116 | Human colon carcinoma |
| MA104 | Monkey African Green kidney |
| BEAS-2B | Human bronchial epithelium, normal |
| NB2-11 | Rat lymphoma |
| BHK 21 (clone 13) | Hamster Syrian kidney |
| NS0 | Mouse myeloma |
| Neuro 2a | Mouse Albino neuroblastoma |
| SP2/0-Ag14 | Mouse × Mouse myeloma, non-producing |
| T47D | Human breast tumour |
| 1301 | Human T-cell leukaemia |
| MDCK-II | Canine Cocker Spaniel Kidney |
| PNT2 | Human prostate normal, immortalised with |
| | SV40 |
| PC-3 | Human Caucasian prostate adenocarcinoma |
| TF1 | Human erythroleukaemia |
| COS-7 | Monkey African green kidney, SV40 |
| | transformed |
| MDCK | Canine Cocker Spaniel kidney |
| HUVEC (200-05n) | Human Umbilical Vein Endothelial Cells |
| | (HUVEC); neonatal |
| NCI-H322 | Human Caucasian bronchioalveolar |
| | carcinoma |
| SK.N.SH | Human Caucasian neuroblastoma |
| LNCaP.FGC | Human Caucasian prostate carcinoma |
| OE21 | Human Caucasian oesophageal squamous |
| | cell carcinoma |
| PSN1 | Human pancreatic adenocarcinoma |
| ISHIKAWA | Human Asian endometrial adenocarcinoma |
| MFE-280 | Human caucasian endometrial |
| | adenocarcinoma |
| MG-63 | Human osteosarcoma |
| RK 13 | Rabbit kidney, BVDV negative |
| EoL-1 cell | Human eosinophilic leukaemia |
| VCaP | Human Prostate Cancer Metastasis |
| tsA201 | Human embryonal kidney, SV40 |
| | transformed |
| CHO | Hamster Chinese ovary |
| HT 1080 | Human fibrosarcoma |
| PANC-1 | Human Caucasian pancreas |
| Saos-2 | Human primary osteogenic sarcoma |
| Fibroblast Growth | Fibroblast Growth Medium Kit |
| Medium (116K-500) | |
| ND7/23 | Mouse neuroblastoma × Rat neurone hybrid |
| SK-OV-3 | Human Caucasian ovary adenocarcinoma |
| COV434 | Human ovarian granulosa tumour |
| Hep 3B | Human hepatocyte carcinoma |
| Vero (WHO) | Monkey African Green kidney |
| Nthy-ori 3-1 | Human thyroid follicular epithelial |
| U373 MG (Uppsala) | Human glioblastoma astrocytoma |
| A375 | Human malignant melanoma |
| AGS | Human Caucasian gastric adenocarcinoma |
| CAKI 2 | Human Caucasian kidney carcinoma |
| COLO 205 | Human Caucasian colon adenocarcinoma |
| COR-L23 | Human Caucasian lung large cell |
| | carcinoma |
| IMR 32 | Human Caucasian neuroblastoma |
| QT 35 | Quail Japanese fibrosarcoma |
| WI 38 | Human Caucasian foetal lung |
| HMVII | Human vaginal maligant melanoma |
| HT55 | Human colon carcinoma |
| TK6 | Human lymphoblast, thymidine kinase |
| | heterozygote |
| SP2/0-AG14 (AC- | Mouse × mouse hybridoma non-secreting, |
| FREE) | serum-free, animal component (AC) free |
| AR42J RAT | Rat exocrine pancreatic tumour |
| PANCREATIC TUMOUR | |

TABLE 2B

| Stressor | Type |
|---|---|
| Concussive force | Environmental |
| Electric shock | Environmental |
| Freezing | Environmental |
| Heat | Environmental |
| High-glucose | Environmental |
| Low-glucose | Environmental |
| Microwave radiation | Environmental |
| Particle radiation | Environmental |
| Ultrasound | Environmental |
| Ultraviolet Light | Environmental |
| X-Ray radition | Environmental |
| Arsenic (As) | Heavy/Transition |
| | metals |
| Cadmium (Cd) | Heavy/Transition |
| | metals |
| Chromium (Cr) | Heavy/Transition |
| | metals |
| Cobalt (Co) | Heavy/Transition |
| | metals |
| Copper (Cu) | Heavy/Transition |
| | metals |
| Iron (Fe) | Heavy/Transition |
| | metals |
| Lead (Pb) | Heavy/Transition |
| | metals |
| Mercury (Hg) | Heavy/Transition |
| | metals |
| Nickel (Ni) | Heavy/Transition |
| | metals |
| Acetic Acid | Industrial Solvent |
| Acetone | Industrial Solvent |
| Acrylonitrile | Industrial Solvent |
| Adipic Acid | Industrial Solvent |
| Aluminum Sulfate | Industrial Solvent |
| Ammonia | Industrial Solvent |
| Ammonium Nitrate | Industrial Solvent |
| Benzene | Industrial Solvent |
| Bisphenol-A | Industrial Solvent |
| Butadiene | Industrial Solvent |
| Butyraldehyde | Industrial Solvent |
| Carbon Black | Industrial Solvent |
| Chlorine | Industrial Solvent |
| Cumene | Industrial Solvent |
| Cyclohexane | Industrial Solvent |
| Ethylbenzene | Industrial Solvent |
| Ethylene | Industrial Solvent |
| Ethylene Dichloride | Industrial Solvent |
| Ethylene Gylcol | Industrial Solvent |
| Ethylene Oxide | Industrial Solvent |
| Formaldehyde | Industrial Solvent |
| Hydrochloric Acid | Industrial Solvent |
| Isobutylene | Industrial Solvent |
| Methanol | Industrial Solvent |
| Methyl tert-butyl ether | Industrial Solvent |
| Nitric Acid | Industrial Solvent |
| Nitrobenzene | Industrial Solvent |
| Nitrogen | Industrial Solvent |
| Oxygen | Industrial Solvent |
| Phenol | Industrial Solvent |
| Phosphoric Acid | Industrial Solvent |
| Potash | Industrial Solvent |
| Propylene | Industrial Solvent |
| Propylene Oxide | Industrial Solvent |
| Sodium Carbonate | Industrial Solvent |
| Sodium Hydroxide | Industrial Solvent |
| Sodium Silicate | Industrial Solvent |
| Styrene | Industrial Solvent |
| Sulfuric Acid | Industrial Solvent |
| Terephthalic Acid | Industrial Solvent |
| Titanium Dioxide | Industrial Solvent |
| Toluene | Industrial Solvent |
| Urea | Industrial Solvent |
| Vinyl Acetate | Industrial Solvent |
| Vinyl Chloride | Industrial Solvent |
| Xylene | Industrial Solvent |
| Bleomycin | Medication |
| Carbon tetrachloride (CCl4) | Medication |
| Doxorubicin | Medication |
| Halothane | Medication |
| Metronidazole | Medication |
| Paracetamol | Medication |
| Antimycin A from Streptomyces sp. | Mitochondrial |
| | inhibitor |
| BMS-199264 hydrochloride ≥98% (HPLC) | Mitochondrial |
| | inhibitor |
| BTB06584 ≥98% (HPLC) | Mitochondrial |
| | inhibitor |
| Carbonyl cyanide 3-chlorophenylhydrazone | Mitochondrial |
| ≥97% (TLC), powder | inhibitor |
| Carbonyl cyanide 4-(trifluoromethoxy) | Mitochondrial |
| phenylhydrazone ≥98% (TLC), powder | inhibitor |
| Lonidamine mitochondrial hexokinase inhibitor | Mitochondrial |
| | inhibitor |
| m-Iodobenzylguanidine hemisulfate salt ≥98% | Mitochondrial |
| (HPLC and TLC) | inhibitor |
| ML-3H2 | Mitochondrial |
| | inhibitor |
| Oligomycin from Streptomyces | Mitochondrial |
| diastatochromogenes ≥95% total oligomycins | inhibitor |
| basis (HPLC) | |
| Pyrrolnitrin from Pseudomonas cepacia | Mitochondrial |
| ≥98% (HPLC), solid | inhibitor |
| Rotenone ≥95% | Mitochondrial |
| | inhibitor |
| TT01001 ≥98% (HPLC) | Mitochondrial |
| | inhibitor |
| α-Cyano-4-hydroxycinnamic acid ≥98% (TLC), | Mitochondrial |
| powder | inhibitor |
| Arsenite | Other Chemical |
| Ethanol | Other Chemical |
| Methyl methanesulfonate | Other Chemical |
| Hydrogen peroxide | Oxidant |
| Hydroperoxyl radical | Oxidant |
| Hydroxyl radical | Oxidant |
| Hypochlorous acid | Oxidant |
| Peroxynitrite | Oxidant |
| Superoxide anion | Oxidant |
| Atrazine | Pesticide |
| Chlorpyrifos | Pesticide |
| Glyphosate | Pesticide |
| Metam sodium | Pesticide |
| Metolachlor | Pesticide |
| Neonicotinoids | Pesticide |
| Paraquat | Pesticide |
| Telone | Pesticide |
| Carbon Dioxide | Pollutant |
| Carbon Monoxide | Pollutant |
| Methane | Pollutant |
| Nitrogen Oxides | Pollutant |
| Ozone | Pollutant |
| Sulfur Dioxide | Pollutant |

The below examples indicate useful extensions and applications of many aspects of the present invention. Although they do not refer to any drawing figure(s) in particular, reference numbers to analogous elements that have previously been introduced in FIGS. 1-6 and described above will be used to aid in the explanations, whenever appropriate.

### Standardized Lab Test Systems

Learning system 100 and method can be applied to standardizing lab test systems for reference bioprocess model 106 when working with biological entities represented by cells or cell lines. Cell lines can be chosen for their ability to model specific conditions or diseases. They can then be subjected to a plurality of stressors that represent a variety of environmental conditions that correspond to various possible local conditions and/or contexts of interest. This matrix of scenarios can be explored in the laboratory by repeated stress and unstressed measurement at standardized intervals to build a more consistent database of time sequences 200 of redox data 112 to be made available to master learner 114. By standardizing the process in this way, a broader range of molecular masses can be measured in a less targeted manner in order to explore the matrix of oxidoreductases and co-factors for features with biological function that may be associated with the system, local conditions, context of interest, and candidate redox indicators that may form the observable basis for a redox status.

### Sensor Fusion Applications

Learning system 100 and method can be applied to the design of field measurement devices 122 by selecting a set of measurements that form an observable basis 116 for a redox status of a bioprocess of interest. One may then combine those measurements into field sensors or sensor fusion systems. Such sensor systems may be initialized with the weights trained by master or local learner 114, 118 and further trained in local contexts according to the method as a soft-sensor model for a sensor fusion device or a combination of stand-alone sensor devices or probes.

### Biological Aging Status Soft Sensor

Defects in the redox system fundamental to metabolism can be a persistent cause of oxidative stress to biological entities. Over time, the stress results in degrading many biological systems in different ways and is inherently related to biological aging status. While there are measures of systemic oxidative stress and chronic inflammation that are associated with aging and chronic disease, these measures look at downstream consequences and stable by products of oxidative stress. It would be advantageous to look instead for any underlying cause(s). One of these may be due to defects causing an imbalance in one or more parts of the redox system in the biological entity of interest.

For example, when misdirected electrons from the redox system form reactive oxygen species. If not reduced by antioxidant such as glutathione, these reactive oxygen species may end up oxidizing proteins, lipids, nucleic acids, and other compounds important to the biological entity and resulting in damage to its system. Oxidized protein products such as amyloid are associated with degenerative diseases such as Alzheimer's. Electrons that oxidize lipids can damage cell membranes and form isoprostanes, MDA and other toxic and carcinogenic compounds. Electrons that oxidize nucleic acids can damage DNA and cause changes to gene expression. Electrons that oxidize small molecules interfere with a wide range of biological processes. Oxidative stress is associated with failure in just about every organ system and disease, particularly chronic diseases and diseases of aging, including but not limited to the heart (CHD, cardiac fibrosis, hypertension, ischemia, myocardial infarction), skin (skin aging, sunburn, psoriasis, dermatitis, melanoma), kidney (chronic kidney disease, renal graft, nephritis), joint (rheumatoid arthritis, osteoarthritis, psoriatic arthritis), lung (asthma, COPD, allergies, ARDS, cancer), brain (Alzheimer's disease, Parkinson's disease, OCD, ADHD, autism, migraine, stroke, trauma, cancer), Immune System (chronic inflammations, autoimmune disorders, lupus, IBD, MS, cancer), blood vessels (restenosis, atherosclerosis, endothelial dysfunction, hypertension), Multi-Organ (diabetes, aging, chronic fatigue), eyes (macular degeneration, retinal degeneration, cataracts).

The use of chronological age as an anchor measure and using learning system 100 and method as described herein with biological samples, redox data and annotations from test subjects at a range of different ages can be advantageous. The data should include healthy subjects and subjects with specific diseases and conditions as listed above, and in combination with other measures that are the downstream consequences and byproducts of oxidative stress, such as chronic inflammation and systemic oxidative stress markers. Using data thus collected, master learner 114 may identify a subset of redox data and its indicators to form an observable basis 116 for chronological age in healthy subjects. To the extent that such a model can be trained by distributed learning algorithm 130 to predict age in healthy subjects, a difference between predicted age and chronological age may be calibrated to represent a “biological age” or “viability” metric in unhealthy or super-healthy subjects. This learning process can be repeated for unhealthy subjects with known diseases and conditions with epidemiologically projected impact on lifespan used as an offset to chronological age in order to calibrate such differences. For subsets of observable measures that also can be collected in the field and also can predict age, a soft-sensor or sensor fusion approach can be provided. In some cases, where field measurement does not yet exist or has not been sufficiently trained for a given context, a biological sample can be collected in the field and sent to the lab for high resolution testing. An initial intake of contextual data determines whether or not a field measurement exists and routes or recommends the sample to a lab that can measure features that quality for observable basis 116.

The system and method can be applied to searching for models that are patterns of measures that regress to an anchor measure of interest, including chronic inflammation and oxidative stress associated with age-related chronic diseases and biological aging in general. To the extent that an observable basis 116 can be identified and trained by distributed learning algorithm 130 to predict the anchor measure, the model becomes a “soft sensor” for that anchor measure. The system and method are first applied using master learner 114 to identify an observable basis 116 for the anchor measure. For example, the anchor measure may be for chronic inflammation and oxidative stress associated with a chronic disease or aging. Local learner 118 is then deployed to determine contexts in which a field-observable subset predicts the same anchor measure. The initial weights are determined by distributed learning algorithm 130 that in this embodiment combines known inflammation and oxidative-stress-related data and redox indicator candidate data into a vector of features for each subject in order to attempt to find weights that regress to the anchor measure.

In the example of an aging model, chronological age of a healthy subject can be used as the anchor measure. Data collected for healthy subjects at a range of ages using full data sets and samples is analyzed in a controlled laboratory environment. This analysis should cover a wide range of known inflammation and oxidative stress markers, sulfur-related redox couples such as ratio of reduced to oxidized glutathione, and a survey of clinical, environmental and behavioral factors believed to have an association with oxidative stress and inflammation such as diet, exercise, sleep, stress, disease diagnoses, injury, medications, environment, and subject history.

Based on this weighting, the aging model would be configured to predict chronological age in healthy subjects by using the weighted model of principal components that regress to natural chronological age in healthy subjects. The initial model would be restricted to the narrow context of the specific healthy test subjects recruited, and could be generalized to the extent that more healthy test subjects are added from broader contexts. For example, in addition to physical health as determined by medical records and recent blood panels, specific contexts include age ranges, gender, ethnic and demographic factors, environmental factors, living conditions, living situation and family, known stressors, psychosocial factors, behavioral factors, cognitive factors, employment, education, family history, DNA and other factors. Any factors known in the literature to be associated with inflammation, oxidative stress, chronic disease, cancer, morbidity or mortality may be exclusions so that the initial model training is on a healthy cohort with no known risk factors.

With unhealthy subjects, to the extent that the same model predicts a deviation from chronological age, this deviation can be used as a metric of “biological age”, “viability” or a combined inflammation and aging status depending on the anchor measures used and success in regressing to that anchor measure within a context. To calibrate this model, well known and well-studied risk factors of inflammation, oxidative stress and aging can be added as cohorts. For example, cohorts of subjects with specific diseases known to have specific links to aging such as diabetes, obesity, hypertension, or specific risk factors such as smoking, sedentary lifestyles, or unhealthy diets may be added. In the other direction, cohorts of subjects who are performance athletes, marathon runners, or other higher than norm performance individuals can be recruited to calibrate for biological age that is younger than chronological age.

Additional metrics such as the HeartAge test from the Centers for Disease Control based on the Framingham Heart Study can be used to calibrate the subject in each cohort. As an example, the Framingham Study Heart Age Calculator from the National Heart Lung and Blood Institute uses gender, chronological age, systolic blood pressure, hypertension treatment, smoking status, diabetes status, and body mass index to predict heart age for people between ages of 30 and 74 who have no history of cardiovascular disease (heart attack, stroke, peripheral artery disease, or heart failure). It is based on the observations that began with 5,209 subjects from Framingham Mass. in 1948 and is now in its third generation of participants.

Measured redox data may contain data associated with the downstream consequences or byproducts of a prolonged imbalance or defect in the redox system. For example, biomarkers of chronic inflammation and systemic oxidative stress, or data related to diseases and conditions that may have a relationship to oxidative stress or inflammation, can be important redox data in many contexts. Many of these biomarkers already have established measurement protocols and some have home or field tests. Examples of systemic oxidative stress measures that can be used with the invention include but are not limited to those found in Table 3A. Examples of chronic inflammation measures that can be used with the invention include but are not limited to those found in Table 3B.

TABLE 3A

| Marker and Type of Damage | Cells | Tissues | Blood | Urine | Other |
|---|---|---|---|---|---|
| DNA/RNA Damage | | | | | |
| 8-hydroxyguanosine (8-OHG) | X | X | X | X | Spinal |
| 8-hydroxydeoxyguanosine | X | X | X | X | |
| (8-OHdG) | | | | | |
| Abasic (AP) sites | X | X | | | |
| BPDE DNA Adduct | X | X | | | |
| Double-strand DNA breaks | X | | | | |
| Comet Assay (general DNA | X | | | | |
| damage) | | | | | |
| UV DNA Damage (CPD, | X | | | | |
| 6-4PP) | | | | | |
| Lipid Peroxidation | | | | | |
| 4-Hydroxynonenal (4-HNE) | X | X | X | | |
| 8-iso-Prostaglandin F2alpha | X | X | X | X | |
| (8-isoprostane) | | | | | |
| Malondialdehyde (MDA) | X | X | X | X | |
| TBARS | X | X | X | X | |
| Protein Oxidation/Nitration | | | | | |
| Protein Carbonyl Content | X | X | X | | |
| (PCC) | | | | | |
| 3-Nitrotyrosine | X | X | X | | |
| Advanced Glycation End | X | X | X | | |
| Products (AGE) | | | | | |
| Advanced Oxidation Protein | X | X | X | | |
| Products (AOPP) | | | | | |
| BPDE Protein Adduct | X | X | X | | |
| Reactive Oxygen Species | | | | | |
| Universal ROS/RNS | X | X | X | X | |
| Hydrogen Peroxide | X | X | X | X | |
| Nitric Oxide | X | X | X | X | |
| Antioxidants | | | | | |
| Catalase | X | X | X | | |
| Glutathione | X | X | X | X | |
| Superoxide Dismutase | X | X | X | | |
| Oxygen Radical Antioxidant | X | X | X | X | Food |
| Capacity (ORAC) | | | | | |
| Hydroxyl Radical Antioxidant | X | X | X | X | Food |
| Capacity (HORAC) | | | | | |
| Total Antioxidant Capacity | X | X | X | X | Food |
| (TAC) | | | | | |
| Cell-Based Exogenous | | | | | Food |
| Antioxidant Assay | | | | | |

TABLE 3B

| Measure | Other Names | Purpose | Sample |
|---|---|---|---|
| Blood Glucose | Blood Sugar; Fasting | To determine if blood glucose | Blood draw, fingerstick, |
| | Blood Sugar; FBS; | level is within a healthy range; | urine sample in some cases, |
| | Fasting Blood Glucose; | to screen for and diagnose | continuous or frequent |
| | FBG; Fasting Plasma | diabetes and prediabetes and to | glucose monitor with |
| | Glucose; FPG; Blood | monitor for high blood glucose | inserted or implanted |
| | Glucose; Oral Glucose | (hyperglycemia) or low blood | sensor some cases. |
| | Tolerance Test; OGTT; | glucose (hypoglycemia); to | |
| | GTT; Urine Glucose | check for glucose in your urine | |
| C-Reactive | CRP | To identify the presence of | Blood draw |
| Protein (CRP) | | inflammation and to monitor | |
| | | response to treatment for an | |
| | | inflammatory disorder | |
| Calprotectin | Fecal Calprotectin; | To detect inflammation in the | Stool sample |
| | Stool Calprotectin | intestines; to distinguish | |
| | | between inflammatory bowel | |
| | | disease (IBD) and non- | |
| | | inflammatory bowel conditions; | |
| | | to monitor IBD activity | |
| Erythrocyte | Sed Rate; | To detect the presence of | Blood draw |
| Sedimentation | Sedimentation Rate; | inflammation caused by one or | |
| Rate (ESR) | Westergren | more conditions such as | |
| | Sedimentation Rate | infections, tumors or | |
| | | autoimmune diseases; to help | |
| | | diagnose and monitor specific | |
| | | conditions such as temporal | |
| | | arteritis, systemic vasculitis, | |
| | | polymyalgia rheumatica, or | |
| | | rheumatoid arthritis | |
| Ferritin | Serum Ferritin | To determine the subject's total | Blood draw |
| | | iron storage capacity | |
| HDL | HDL; HDL-C; High- | Monitoring at regular intervals | Blood draw or from a |
| Cholesterol | density Lipoprotein | as part of a lipid profile when | fingerstick |
| | Cholesterol | risk factors for heart disease are | |
| | | present, when prior results | |
| | | showed high risk levels, and/or | |
| | | when undergoing treatment for | |
| | | unhealthy lipid levels | |
| High-sensitivity | hsCRP; High- | To help assess your risk of | Blood draw |
| C-reactive | sensitivity CRP; Ultra- | developing cardiovascular | |
| Protein | sensitive CRP; Cardiac | disease | |
| | CRP; CRP for heart | | |
| | disease | | |
| Homocysteine | Plasma Total | To help determine folate or | Blood draw, sometimes |
| | Homocysteine; Urine | vitamin B12-deficiency; to | urine sample |
| | Homocysteine; | determine increased risk of | |
| | Homocysteine Cardiac | heart attack or stroke; to help | |
| | Risk | diagnose a rare inherited | |
| | | disorder called homocystinuria | |
| Interleukin-6 | IL-6 | To help evaluate conditions | Blood draw |
| | | such as diabetes and | |
| | | cardiovascular disease or | |
| | | conditions associated with | |
| | | inflammation such as lupus and | |
| | | rheumatoid arthritis or with | |
| | | infection, such as sepsis | |
| Lactoferrin | Fecal Lactoferrin; | To detect inflammation in the | Stool sample |
| | Stool Lactoferrin; | intestines; to help identify | |
| | Fecal WBC Non- | active inflammatory bowel | |
| | microscopic | disease (IBD); to distinguish | |
| | | between IBD and non- | |
| | | inflammatory bowel conditions; | |
| | | to monitor IBD activity | |
| White Blood | WBC Count; Leukocyte | To screen for or diagnose a | Blood draw or by a |
| Cell Count | Count; White Count | variety of conditions that can | fingerstick or heelstick |
| | | affect white blood cells (WBC) | |
| | | such as an infection, | |
| | | inflammation or a disease that | |
| | | affects the production or | |
| | | survival of WBCs; to monitor | |
| | | treatment of a blood disorder or | |
| | | therapy that is known to affect | |
| | | WBCs | |

### Chronic Inflammation

Learning system 100 and present methods can be applied to the identification and calibration of patterns of measures for inflammation that include subjective and self-assessed measures and contextual cues. Inflammation is a normal immune response to injury, including trauma, bacterial or viral infection, burns including sunburn, chemical irritants, frostbite, cuts in the skin, and allergic reactions. Pain, swelling, redness, and warmth are all signs of inflammation arriving at the site of an injury and are the first step in the healing process. Acute inflammation is a brief inflammatory response to an injury or illness that only lasts a few days. Inflammation becomes chronic when the acute response is no longer necessary but a constant low-level physiological response remains. With chronic inflammation, the organism no longer has the ability to turn off the inflammatory response, and the inflammatory response designed to clear out damage starts to cause more damage to healthy tissues. Examples include damaging the intestinal lining of the gut and causing inflammatory bowel disease such as ulcerative colitis and Crohn's disease, damaging the lining of the stomach and causing chronic peptic ulcers, damaging the mucus membranes of the sinuses and causing chronic sinusitis, damaging the gums and causing chronic periodontitis, damaging arteries and causing coronary artery disease and atherosclerosis, damaging the tissues in the joints and causing rheumatoid arthritis, damaging structures in the skin and causing eczema, rosacea, seborrheic dermatitis, and psoriasis, damaging the lungs and causing asthma, chronic obstructive pulmonary disease (COPD), and pulmonary fibrosis, and many other systems. Chronic inflammation also is associated with chronic neurodegenerative diseases such as Alzheimer's disease and Parkinson's disease, and has been associated with the emergence of many cancers.

The five classic signs of acute inflammation from an injury or insult close to the skin and the peripheral nerves have been recognized in medicine for over 2,000 years, and can be remembered by the modern acronym PRISH:
Pain—the inflamed area is likely to be painful, especially when touched. Chemicals that stimulate nerve endings are released, making the area much more sensitive.
Redness—this is because the capillaries are filled up with more blood than usual
Immobility—there may be some loss of function
Swelling—caused by an accumulation of fluid
Heat—as with the reason for the redness, more blood in the affected area makes it feel hot to the touch.

In 1992, the American College of Chest Physicians (ACCP) and the Society of Critical Care Medicine (SCCM) introduced definitions for systemic inflammatory response syndrome (SIRS), sepsis, severe sepsis, septic shock, and multiple organ dysfunction syndrome (MODS). The idea behind defining SIRS was to define a clinical response to a nonspecific insult of either infectious or noninfectious origin. SIRS is defined as 2 or more of the following variables:
Fever of more than 38° C. (100.4° F.) or less than 36° C. (96.8° F.)
Heart rate of more than 90 beats per minute
Respiratory rate of more than 20 breaths per minute or arterial carbon dioxide tension (PaCO2) of less than 32 mm Hg
Abnormal white blood cell count (>12,000/μL or <4,000/μL or >10% immature [band] forms)

SIRS is nonspecific and can be caused by ischemia, inflammation, trauma, infection, or several insults combined. Thus, SIRS is not always related to infection, but it has the advantage that three of the four variables in the model can be readily and accurately measured by home monitoring devices.

When inflammation is chronic and especially when it is deeper in the body, the signs are less specific and can be harder to recognize. Many subjective markers associated with chronic inflammation can be assessed at home or monitored more directly by subjects themselves:
High blood pressure or blood sugar problems
Flare-up of autoimmune conditions: This includes sore joints, ongoing or irritating muscle pains, dry, patchy, and/or red skin, bloodshot eyes, allergies and asthma.
Water retention: Where acute inflammation is often characterized by swelling at the site of injury, systemic inflammation can result in a non-localized water retention.
Gastrointestinal problems and disturbances such as ulcers, constipation, diarrhea, including irritable bowel syndrome.
Stress load: While stress is highly individual and subjective, there are common indicators of stress such as rubbing your temples, face palming, frequent sighing, and pinching the space between your eyes.
Persistent unexplained nasal congestion: Could be related to allergies, hay fever and food allergy, which also may be exacerbated by other inflammation.
Overtraining: Exercise causes inflammation and if done in excess of what the body is ready for or without proper recovery time, this inflammation can become chronic.
Constant feeling of fatigue or lethargy, a subjective measure that can become an essential metric with consistent self-assessment over time. More specific questions can make this metric more concrete as a measurement.

Even if these metrics are subjective and not calibrated to a gold standard, as long as the subject is consistent, such inputs may be included in redox data according to the system and method for consideration as part of an overall pattern of data that could be part of an inflammation measurement. Taken alone, any subjective measure could be a non-specific or harmless artifact, but in combination with other measures they could become an important component of an overall soft-sensor indicator.

The most common way of measuring inflammation is the blood test for CRP or C-Reactive Protein. CRP is a protein produced in the liver that binds with phosphocholine on dead and dying cells and bacteria in order to clear them from the body. With the acute inflammation caused by infection, for example, CRP can spike by up to 50,000-fold. CRP spikes due to acute inflammation peak at around 48 hours and decline pretty quickly thereafter, with a half-life of about 18 hours after the acute phase inflammation peak. With an acute inflammation from an injury, trauma or pathogen, CRP goes back to normal a few days after the incident is resolved. If CRP persists, the injury, infection or trauma probably also persists.

CRP is highly sensitive to many different kinds of stressors, and elevates in response to anything that causes inflammation. It is a valuable marker determining that inflammation is occurring, but it is not specific, so it is difficult to impossible to determine why the inflammation is occurring. Still, CRP is considered an independent predictor of high risk for coronary artery disease. According to the American Heart Association and the Centers for Disease Control and Prevention, a CRP concentration of below 1.0 mg/L indicates low risk for heart problems; between 1.0 to 3.0 mg/L is an average risk for heart problems; above 3.0 mg/L as high risk for heart problems. Very high levels of CRP (more than 10 mg/L) can also indicate impaired immune response or inflammatory disease. If the measurement is over 1.0 mg/L in the absence of any acute stressors, chronic, other sources of systemic inflammation could be the cause. Note that exercise can be a stressor that causes a temporary rise in CRP, as can be pregnancy, so context is an important factor.

White blood cell (WBC) or leukocyte count also is a measure associated with inflammation. White blood cells are an essential agent of the body's immune system and the body produces more when body senses a foreign threat in the bloodstream. A high WBC count (considered to be 10,500 leukocytes per microliter of blood in most labs) can indicate an infection, stress, inflammation, trauma, allergy, or presence of certain diseases, while a count of 4,500-10,500 is within the normal range.

CRP is produced by the liver and increases following the interleukin-6 (IL-6) secretion by T Cells, a type of white blood cell that plays a huge role in the immune response, and macrophages, cells that engulf and digest stray tissue and pathogens. Because both T Cells and macrophages secrete IL-6 as part of the inflammatory response, an elevated IL-6 can indicate systemic inflammation. Other measurements of markers of inflammation are well established in medicine in addition to C-Reactive Protein, White Blood Cells, and Interleukin-6. Most are measured from a blood sample in a lab, but some are accessible with small samples from a finger-stick. Other measures also are found in the stool, urine and other fluids.

The above referenced biomarkers and subjective or self-assessed measures may be included in the redox data for test subjects providing data to learning system 100 for two purposes. First, this data may be used to define or narrow a context in which the learned model can apply. Second, to the extent that the above measures are related to redox status or are correlated with redox status measures, they may be selected as features of the model itself. This becomes more important when such a measure is available or easier to measure in the field than alternative features.

The full set of markers that forms an observable basis 116 for predicting biological age or combined inflammation-aging status available in a lab environment may not be available or practical in the home or field environment (i.e., under local conditions and context). The invention further adjusts the weights with a conditioning module that can further weight observable measures or exclude them based on the availability in the home or field environment, or based on practicality of home or field measurement. An observable basis 116 that also can be measured in the home or field environment may be restricted at first only to narrow contexts or may be very imprecise because of insufficient data to calibrate the home or field measurement. Noting the limitations, the objective is to provide a method to systematically improve the home or field prediction model through local learner 118 that is connected with master learner 114.

Master learner 114 provides local learner 118 with an initial set of weights based on reference bioprocess model 106 that also reflects the cohorts of subjects studied in the lab environment. Local learner 118 then calibrates or trains based on the contextual data and local field measurements. The greatest limitation to useful monitoring in the field is that precise measurement of factors known to be associated with oxidative stress, inflammation and aging are non-specific and difficult to measure in a consistent manner. The invention can be used in applications that address this issue by training both master learner 114 and local learner 118 with the inclusion of passive data sources that are indicators of lifestyle, exercise, diet, disease, and behavioral factors. This includes the direct measurement of activity from wearable devices, the measurement of psychosocial factors and behaviors from social media models, and the measurement of dietary factors from credit card and loyalty card data, and if available, from smart refrigerators or in-home smart assistants like Amazon Alexa and others.

As learning system 100 accumulates a pattern of data associated with oxidative stress, inflammation and aging, including inputs from consumer mobile and social media devices, the system also can be applied to recommending changes to these same behavioral inputs. This can be in the form of a recommendation to the user, or in the form of formulation of medical foods or nutritionals, vitamins or supplements, or inputs to the grocery basket of an online food ordering and delivery service such as Blue Apron.

The system and method described above can be applied in several areas with lab test systems and field measurement approaches that yield more specific data of interest to a therapeutic area or application. This includes but is not limited to the following:

### Improved Patient Monitoring Systems.

The system and method can be applied to identifying and training soft-sensor models of oxidative stress that are coupled to or incorporated in patient monitoring systems including oxygen concentration, oxygen consumption rate, glucose concentration, glucose consumption rate and combinations thereof. In vivo measurement at the beginning of energy metabolism process, blood glucose, and at the end of the metabolic process, blood oxygen, are standards of care for many conditions. However, many of the metabolic steps in between remain a “black box”. It is not completely a black box, because we know generally how the system works and we can measure some individual features of that system. 95% of the electrons from the glucose source flow through the electron transport chain to generate cellular energy before ending up in the oxygen sink. 4%-5% of the electrons flow to three systems that are pillars of the antioxidant system responsible for cleaning up the toxic byproducts of cellular respiration and regulating homeostasis in cells. These pillars are Glutathione, Thioredoxin, and Cysteine, all part of the sulfur metabolome. Although the individual components may vary and there may be many alternative pathways that can account for specific measures in the system, there are more specific and more predictive nodes in the network, and the overall balance of reduced sulfur to oxidized sulfur is related to the overall oxidative stress in the system.

Even if biomarkers of energy metabolism can be measured in vivo from blood, plasma, urine, breath, sweat, saliva or other fluids, these biomarkers may lose essential information about their source, such as a specific organ system, injury or infection. The accepted processes of developing clinically validated measurements are further complicated by the calibration of the result because of the fact that the cellular energy system is so adaptable and responsive to environmental variations and stresses. It has been difficult to measure many of the more specific nodes or redox indicators with precision and specificity in vivo because the measurements of interest are in hidden compartments and hard-to-reach systems, and are parts of a complex dynamic network that has evolved to adapt to a wide range of environmental variation, making any measurement highly context dependent. This leads to a Catch-22 situation where the only way to learn and validate surrogate measures may require us to deploy measurement at scale in real-world situations to observe patterns of measures in context, but clinical practice generally will not allow the deployment of such measurements until after they are validated.

The present system and method may be applied under conditions that combine validated measurements of blood oxygen concentration, oxygen consumption rate, and/or blood glucose with candidate redox indicator measurements and other patient data and clinical annotations. This combined vector of redox data would be provided to the learning system according to the invention to identify an observable basis for the internal state of hidden compartments and hard-to-reach systems by training the learning model with a large number of observed patient state vectors that include labeled data from more precise laboratory systems and clinical annotations that relate to the internal state.

The measurement of the electron source, glucose, and the electron sink, oxygen, constrain the possible states available to the system. Blood glucose and total glucose consumption rate, and blood oxygen and total oxygen consumption rate, can be directly measured. Because cellular respiration involves electron flow from glucose to oxygen, these measures can provide strong constraints on the overall metabolic model based on known chemistry, which can be calibrated for the whole person based on a known set of inputs including weight, nutrient intake, and other standard measures.

Clinical annotations and medical records and laboratory systems with more precise measurement capabilities serve as a source labeled data that correspond to specific diagnoses and organ systems. Based on this labeled data, collected from a large number of patient monitoring systems and labs, the system and method can be applied to identifying and training a set of redox indicators of the internal state that more closely associate with specific organ systems.

### Medical Foods and Vitamin E Application.

An inherently tunable part of nature's system to regulate balance and maintain a stable state of health in a living system—and a source of potential environmental variation and disturbance—is food. Other tunable inputs include lifestyle factors such as exercise, sleep, stress, living situation, relationship status, stress mitigation activities including meditation, and other behaviors. Biological entities in nature have evolved as part of food systems or networks that provide a complex cocktail of nutrients and behaviors that comprise many of the tunable inputs that maintain homeostasis. After billions of years of natural evolution of these complex networks, human activity has begun to disrupt these networks in unprecedented ways that are not well understood and have led to a rise in chronic diseases in humans and other biological entities, and the instability or even collapse of natural ecosystems.

Some of the simpler dietary inputs have been observed for decades, such as the observations that led to the discovery of Vitamin E in 1922: Rats given a simple diet of carbohydrates, fats and proteins with no vegetables became sterile. Fertility was restored once green leafy vegetables were reintroduced into the diet, leading to the hypothesis that there must be a mystery substance in such vegetables. Vitamin E turned out to be a more complex system of molecules with a number of different forms with different functional outcomes. Of course, food and nutrient balances include a far more complex set of inputs, and a method is needed to uncover and tune a much larger number of parameters to regulate a complex living system in a healthy state for a longer period of time, or to compensate for a growing number of environmental disturbances and insults. Even then, the regulation or control of a complex system still may be limited to specific contexts. The methods presented herein may be applied to learning which subsets parameters can form an observable basis for status of a hidden state in a complex biological entity, and for learning which sets of tunable parameters can be used to regulate a complex biological entity, and can be applied to further improve the measurement and regulation of complex biological entities by learning the contexts in which they apply.

Other subjective measures related to systemic oxidative stress, inflammation and aging which also are potential tunable inputs that in addition to specific diet inputs can be part of a control recommendation from the system include but are not limited to:
Avoiding processed foods that are high sugar, high carbohydrate, high fat, high gluten or high protein from animals that have been subject to concentrated artificial feeding.
Increasing omega-3 and reducing omega-6 intake: Omega-3 fats form the precursors for anti-inflammatory eicosanoids, while Omega-6 fats form the precursors of inflammatory eicosanoids, both of which are part of the inflammatory response. A high ratio of omega-6 to omega-3 fats can produce and imbalanced inflammatory response to normal stimuli.
Improving sleep: Poor or insufficient sleep is linked to elevated inflammatory markers and is a chronic problem especially in developed or urban environments.
Exercising more: In modern societies, many people tend to lead sedentary lives, and this lack of activity is linked to systemic, low-grade inflammation.
Allowing recovery time: Overtraining with too little rest and recovery can produce chronic inflammation.
Mitigating chronic stress: Modern life is stressful and emotional stress has a cumulative effect inflammatory response. This response is compounded by being “always on” without downtime or time in nature that allows the body to recharge.
Improving gut health: The gut houses the bulk of the human immune system which is regulates inflammation, and contains an entire microbiome of organisms that participate in the process.

The food-related inputs to a subject can be measured with a variety of self-reporting devices such as mobile or wearable food loggers. Automated or semi-automated reporting data can be gathered from smart refrigerators or food storage systems that report consumption data and may be accessed directly or via an application program interface. For institutional settings served by a food service operator, restaurant chain, or cafeteria, as well as most agricultural settings where nutrition is provided in an industrialized and planned manner, the meal or nutritional plan and ingredient data can be captured from meal or nutritional planning systems. In addition, generalized information about food consumption patterns can be obtained automatically through purchase behaviors including credit card and loyalty card behaviors. Depending on the precision and confidence in measurement, this food data may be binned based on detailed ingredients and cross-referenced with food databases, or based on more general classifications such as high versus low consumers of categories of food associated with health and redox status, such as fresh fruits and vegetables, red meat, or sugary drinks. Recommendations to changes in tunable inputs such as food choices, composition, vitamins or nutritional supplements be presented to the consumer, shopper or caregiver, or can be implemented automatically in food formulation systems, supplement formulation systems, medical foods, food delivery systems, meal kits and the like. These additional inputs of redox data and annotations can be applied to systems aiming to enhance regulation and control for a wide range of consumer and clinical use cases involving consumer food, medical food, nutritionals, vitamins and supplements.

### Skin Care Applications.

The system and method can be adapted for skin care applications that incorporate skin-specific forms of measurement of redox data and annotations that complement sensor or chemical measurement of redox data. One embodiment includes a self-reported skin assessment in combination with mobile imaging of skin regions such as face, blemishes, rashes or other areas of interest. These images may be classified and scored based on skin assessment data using automated machine learning methods to provide data with increasing structure related to skin conditions and skin care. In addition, control inputs related to skin can be measured through purchase behavior monitoring, self-reporting, and also through direct measurement, or measurement of subject location from a smartphone or other location measurement system and access to location-based databases with solar radiation or UV data by location.

### Neuro-Degenerative Diseases and Mental Health Applications.

The system and method can be applied to systems targeting redox balance and oxidative stress associated with many neuro-degenerative diseases including Parkinson's Disease, Alzheimer's Disease, depression, anxiety, attention deficit and other conditions that have been difficult to measure especially in their earlier days. In addition to measuring dietary and lifestyle inputs through self-reporting, mobile or wearable devices, and monitoring of purchase behaviors, important metrics of neuro-degenerative diseases and mental health conditions can be yielded from social media behaviors and communications data alone or in combination with other activity data, including sentiment analysis and classification of communications.

### Diabetes and Metabolic Syndrome Monitoring and Management Systems.

The system and method can be applied to systems for the management of diabetes and metabolic syndrome. In combination with blood glucose monitoring, insulin delivery systems, including implantable insulin delivery devices, continuous blood glucose monitors, and closed-loop systems, and other regiments aimed at improving the monitoring and management of blood glucose in diabetes and metabolic syndrome or pre-diabetes, the above teachings can be combined with existing glucose monitoring regimens to improve the management and care of subjects. Blood glucose and related analytes can be an important redox-related field measurement especially in combination with measurement and control of diet and lifestyle inputs which may be supplemented by the diet and behavioral measurement described above.

### Industrial Biology Applications.

The systems and methods herein may be implemented in a system designed as a bioreactor monitoring and control agent in which existing data on bioreactor operational status accessed via a direct connection or application program interface. Control signals to the bioreactor with respect to a specific ingredient or combination of ingredients or controls can be transmitted to the operating control system for the bioreactor via a direct connection or application program interface. Examples include increasing or decreasing a specific enzyme used in bioreactor production to extend the productive stationary phase based on monitoring redox status signals.

### Agriculture Applications.

The systems and methods herein may be applied to agriculture management applications designed to improve the feeding, nutrition and management of livestock and other animals used for food, food production, and other purposes. They may be used for crop nutrition, fertilization, and management in the same manner. The methods also can be deployed to identify an observable basis for systemic health status of agricultural land, ecosystems and food webs when an anchor measure of such systems can be described, such as productive yield or other measures of health and productivity.

### Redox-Related Context Adjustments to Bioprocess

Computer implemented learning methods, systems and their various applications described above or deployed in accordance with the teachings of the invention can further benefit form contextual information. Specifically, discovering or learning about redox-related context adjustments to biological processes as experienced under local conditions is very advantageous. In discussing systems and methods for context discovery we will refer to previously introduced parts and their analogues by using the same reference numbers whenever practicable.

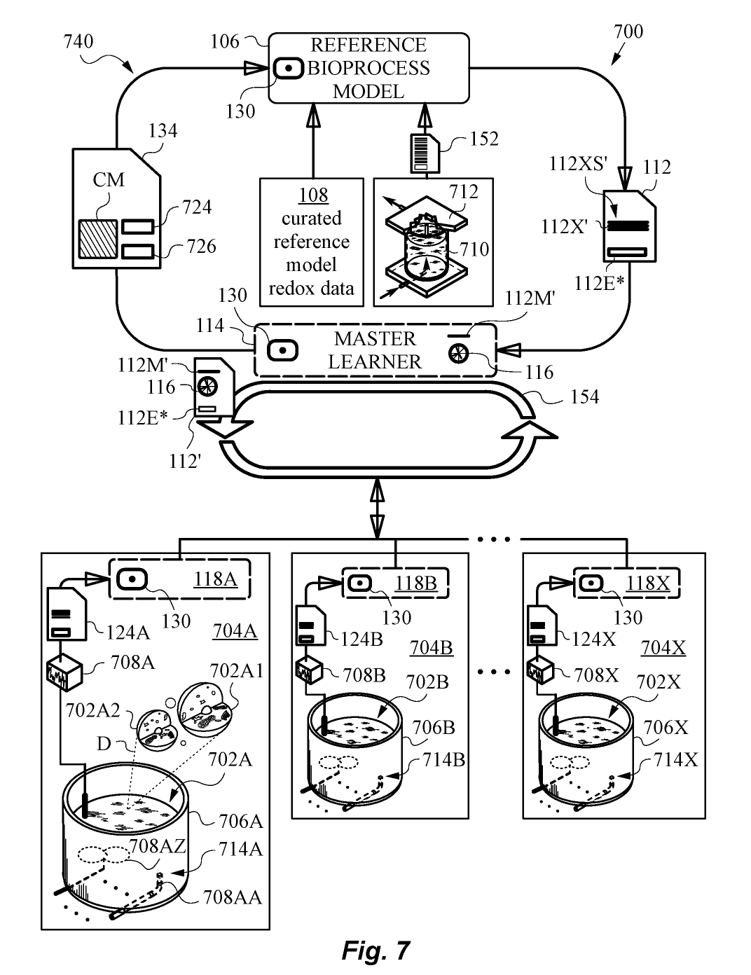

FIG. 7 is a diagram illustrating a learning system 700 configured to learn a redox-related context adjustment to a bioprocess experienced by one or more biological entities under local conditions. In the present example, a number of biological entities 702A, 702B, . . . , 702X are embodied by cell lines. A couple of individual cells 702A1, 702A2 of cell line 702A are shown in an enlarged or magnified section D. Each cell line 702A, 702B, . . . , 702X is in a different physical location and under its own local conditions 704A, 704B, . . . , 704X inside its own bioreactor 706A, 706B, . . . , 706X. It is understood that appropriate media, matrices, additives and other materials are typically provided inside bioreactors 706A, 706B, . . . , 706X to support cell lines 702A, 702B, . . . , 702X.

While inside their bioreactor 706A, 706B, . . . , 706X, each cell line 702A, 702B, . . . , 702X experiences the bioprocess that involves redox reactions. The bioprocess transpiring in each bioreactor 706A, 706B, . . . , 706X is sensed, monitored and/or measured by a corresponding sensor system 708A, 708B, . . . , 708X. Although not explicitly shown, each sensor system 708A, 708B, . . . , 708X may include one or more individual measurement devices, sensors and/or monitors as well as any requisite interfaces, hardware and software. Sensor systems 708A, 708B, . . . , 708X gather or collect measured redox data 124A, 124B, . . . , 124X generated by biological entities 702A, 702B, . . . , 702X as they experience the bioprocess under their own local conditions 704A, 704B, . . . , 704X inside their own bioreactors 706A, 706B, . . . , 706X.

Learning system 700 uses a highly distributed learning architecture in which each sensor system 708A, 708B, . . . , 708X communicates directly with its local learner 108A, 108B, . . . , 108X. Specifically, each sensor system 708A, 708B, . . . , 708X provides measured redox data 124A, 124B, . . . , 124X that it has collected to its local learner 108A, 108B, . . . , 108X. In turn, each local learner 108A, 108B, . . . , 108X runs distributed learning algorithm 130 on its local resources. Local learners 108A, 108B, . . . , 108X are connected to master leaner 114 via primary feedback loop 154.

In learning system 700 of the present example, reference bioprocess model 106 is built from both curated reference model redox data 108 and model redox data 152 measured or collected from a reference biological entity 710. Curated reference model redox data 108 resides in an annotated, classified and labeled database built up form past tests. It is connected directly to reference bioprocess model 106 and, in the absence of other data, can serve as the sole source of curated data for constructing reference bioprocess model 106.

In the present example, reference biological entity 710 is a model or reference cell line that is undergoing the bioprocess under model conditions in a reference bioreactor 712. Cell line 710 resides in an appropriate medium within reference bioreactor 712 for undergoing the bioprocess that involves redox reactions under model conditions. To maintain model conditions, the environment both outside and inside reference bioreactor 712 is preferably well controlled. Specifically, bioreactor 712 is housed within a controlled facility such as a laboratory (not shown).

Model cell line 710 is also provided with nutrients and inputs necessary to undergo the bioprocess in vitro. Note that cell line 710 can be chosen for its ability to model specific conditions or diseases. Cell line 710 may be chosen from among immortalized cell lines or cell lines specific to certain biological entities of interest.

The present computer implemented learning system 700 learns about redox-related context adjustments to the bioprocess with the aid of reference bioprocess model 106. Reference bioprocess model 106 is used to describe the bioprocess as experienced by reference biological entity 710, in which it is considered as the reference bioprocess. Of course, the bioprocess is also experienced by local biological entities 702A, 702B, . . . , 702X that undergo the bioprocess under their own field or local conditions 704A, 704B, . . . , 704X.

As in the previous embodiments, redox status even under model conditions, is considered as indirect, inferred or otherwise derived knowledge. Correspondingly, reference bioprocess that reference biological entity 710 undergoes is postulated to have hidden states that are not directly observable. The hidden states may, and in typical embodiments of the present invention will, include unknown states beyond those of just the redox status of the bioprocess that the reference biological entity or local biological entity is experiencing.

The bioprocess from which learning system 700 learns or on which it can be trained is reference bioprocess model 106. The hidden states are a part of reference bioprocess model 106. Reference bioprocess model 106 is designed to provide, output or yield model redox data 112 along with a preliminary, initial or reference learning model.

In the present example, model redox data 112 contains the first four redox categories 112A-D already collapsed into one joint redox category 112X. Joint redox category 112X corresponds to joint redox category already introduced in above embodiments. However, joint redox category 112X for reference bioprocess model 106 typically contains all available data. In previous embodiments, on the other hand, joint redox category 112X may have been downscaled or pruned in light of their relevancy to local biological entities 702A, 702B, . . . , 702X and local conditions 704A, 704B, . . . , 704X within their bioreactors 706A, 706B, . . . , 706X where they experience the bioprocess.

All redox indicators are organized in the single or joint feature vector 112X′. One joint feature vector 112X′ in the time series 112XS' is specifically called out in FIG. 7. Contextual data contained in model redox data 112 is presented in the form of contingency list 112E*.

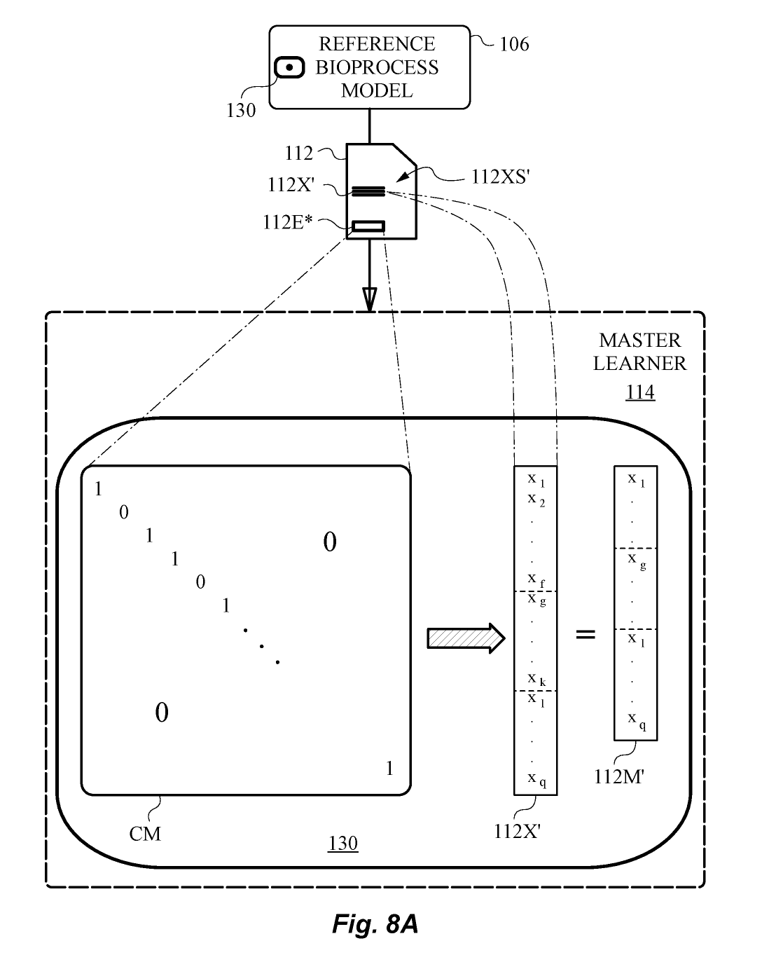

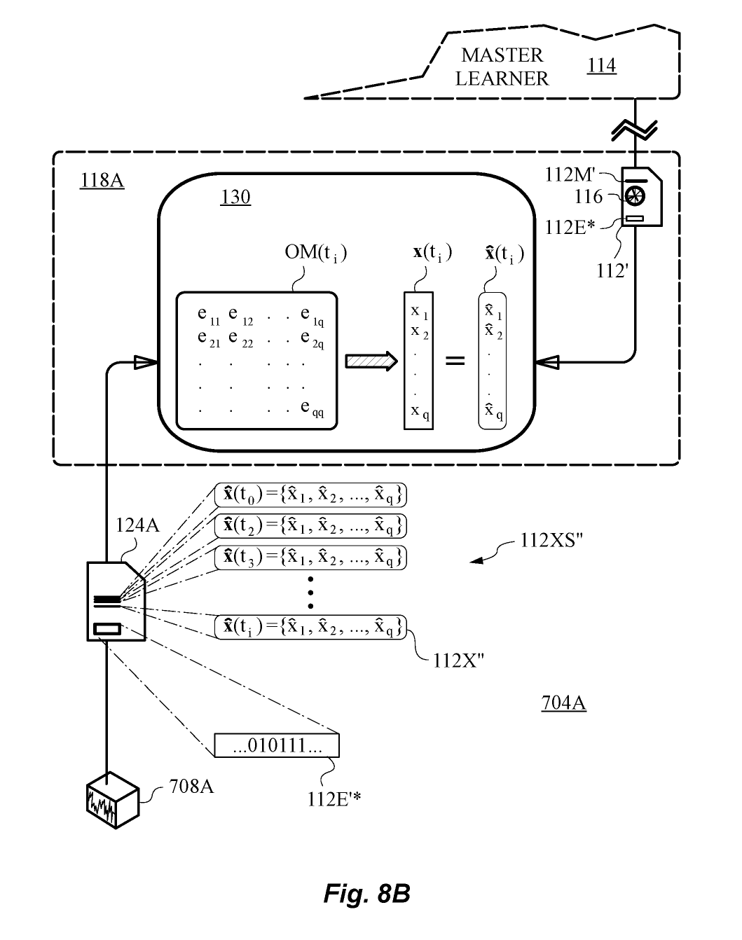

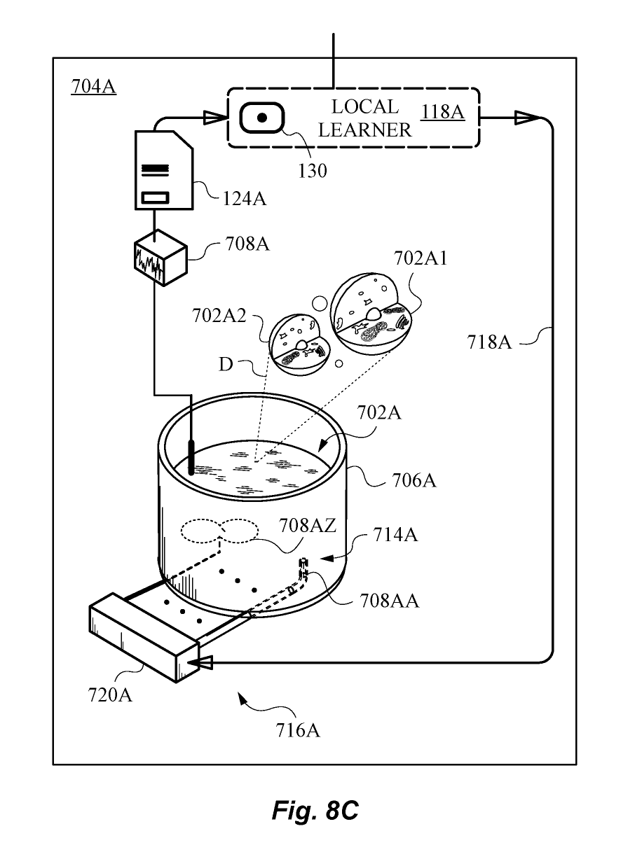

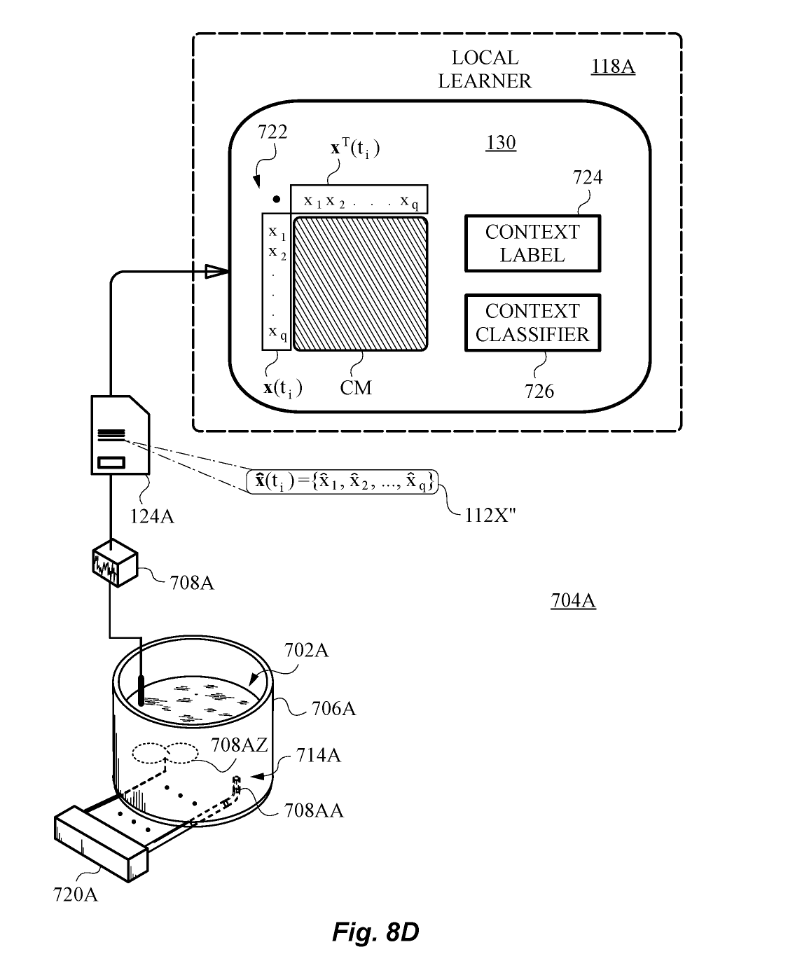

FIG. 8A is a diagram that illustrates the entries of joint feature vector 112X′ and the contextual data in contingency list 112E* in more detail. Specifically, contingency list 112E* contains a context matrix CM that is judged appropriate based on reference bioprocess model 106 for the known or expected local conditions 704A, 704B, . . . , 704X under which local biological entities 702A, 702B, . . . , 702X are undergoing the bioprocess. In some embodiments, contextual data in contingency list 112E* may actually contain a selection of context matrices appropriately labeled and ordered according to estimated, expected or known local conditions 704A, 704B, . . . , 704X. In those embodiments master learner 114 may select and event test for the most appropriate context matrix CM. Further, master learner 114 could be aided in making this selection based on information received from any one or more local learners 108A, 108B, . . . , 108X.

Distributed learning algorithm 130 running in master learner 114 uses the most appropriate context matrix CM by applying it to joint feature vector 112X′. This operation transforms joint feature vector 112X′ to a model feature vector 112M′. In the present example, context matrix CM is a simple diagonal matrix that either keeps or drops entries of joint feature vector 112X′ during the transformation. In other words, context matrix CM belongs to the family of projection matrices. Specifically, we note that second entry x₂ corresponding to the second redox indicator as well as the ones associated with entries x_{f}, xₖ and their redox indicators are dropped in model feature vector 112M′. A person skilled in the art will recognize that any number of more complicated transformations can also be encoded by context matrix CM. For example, the function of conditioning module 210 already introduced above, could be incorporated into context matrix CM by including re-scaling of certain entries, applying weighting factors or functions as well as other types of data conditioning.

Thus, from joint feature vector 112X′ and contingency list 112E* as delivered, master learner 114 obtains model feature vector 112M′. Model feature vector 112M′ has the appropriate form given the local conditions 704A, 704B, . . . , 704X under which local biological entities 702A, 702B, . . . , 702X are undergoing the bioprocess. Of course, if the local conditions for a specific local biological entity among cell lines 702A, 702B, . . . , 702X are different from the other ones, the form of model feature vector 112M′ for those local conditions may be obtained with a different context matrix CM and may thus present a still different form.

In addition, master learner 114 is configured to establish from the information received in model redox data 112 an observable basis of redox indicators 116. In the present case, given that a number of entries were dropped by context matrix CM from joint feature vector 112X′ to yield model feature vector 112M′, finding observable basis of redox indicators 116 will involve additional steps. These may involve renormalization and additional operations required due to the reduction in the dimensionality of model feature vector 112M′ from the full dimensionality of joint feature vector 112X′. Persons skilled in the art will be familiar with these types of operations and adjustments. Corresponding instructions are preferably included as part of data in contingency list 112E* or elsewhere within model redox data 112 (not expressly shown).

Observable basis 116 excludes any hidden states or otherwise hidden or inaccessible data. Thus, any vector spaces established using observable basis of redox indicators 116 are real-valued and measurable. Any candidate redox indicators in such vector spaces can be assigned real values and measured. The process for establishing observable basis 116 has already been taught above.

As expressed in observable basis 116, we will continue to refer to the context-adjusted joint feature vector 112X′ as model feature vector 112M′. Thus expressed, model feature vector 112M′ can be considered to be in canonical form. When model feature vector 112M′ is expressed in canonical form and is also obtained in baseline redox-related context that has not been disrupted or adjusted we consider model feature vector 112M′ to be in the initial state. Those skilled in the art may also refer to this situation as vector representation under initial model conditions or under ideal conditions.

It is important to obtain the canonical form of model feature vector 112M′ in observable basis 116 while reference bioprocess model 106 is in baseline redox-related context. Perturbations will cause model feature vector 112M′ to depart from its canonical form. The ways in which model feature vector 112M′ changes from its canonical form can then be associated with the perturbation or change in model conditions within reference bioreactor 712. In the present case, however, we are concerned with local biological entities 702A, 702B, . . . , 702X and how they experience the bioprocess. Hence, reference bioprocess model 106 does not involve applying intentional perturbations to model conditions or redox-related context adjustments to model 106.

Referring back to FIG. 7, we see that a portion of model redox data 112′ contains model feature vector 112M′ in observable basis 116 and data specific to local conditions 704A, 704B, . . . , 704X, namely contingency list 112E*. In other words, in the present embodiment master learner 114 does not send all model redox data 112 to local learners 118A, 118B, . . . , 118X via primary feedback loop 154. Instead, primary feedback loop 154 transmits just portion 112′ of model redox data 112 that contains model feature vector 112M′ obtained with the aid of context matrix CM. Of course, portion 112′ can include the full or almost full set of model redox data 112 when local learners 118A, 118B, . . . , 118X are deployed with ample computing resources and dispose of significant communication bandwidth for receiving data.

Local learners 118A, 118B, . . . , 118X also receive the full set of measured redox data 124A, 124B, . . . , 124X generated by biological entities 702A, 702B, . . . , 702X inside their bioreactors 706A, 706B, . . . , 706X. Portion 112′ of model redox data 112 and local measured redox data 124A, 124B, . . . , 124X are used by each local learner 108A, 108B, . . . , 108X to learn. Specifically, local learners 108A, 108B, . . . , 108X deploy their local distributed learning algorithm 130 to learn the relationship between model feature vector 112M′ that has been adjusted for local conditions 704A, 704B, . . . , 704X by the application of context matrix CM in master learner 114 and the locally obtained measured redox data 124A, 124B, . . . , 124X. Note that as in the previous embodiments, model redox data 112 and/or its portion 112′ sent to local learners 118A, 118B, . . . , 118X may contain an initial reference learning model and any initial weights or starting points specifically intended for local learners 118A, 118B, . . . , 118X. Of course, some of these can be accounted for by context matrix CM, as appropriate.

FIG. 8B is diagram illustrating in more detail the learning performed by one of local learners 118A, 118B, . . . , 118X, namely learner 118A that is tracking the bioprocess under local conditions 704A. Sensor system 708A is shown here sending to local learner 118A measured redox data 124A. The latter contains measured contextual or list redox data 112E′* and a measured time series 112XS″ of individual measured joint feature vectors 112X″. The actual vectors indicated in FIG. 8A use the hat notation as another reminder that their entries are actual measured rather than model values.

Upon receipt of each one of joint feature vectors 112X″ of measured time series 112XS″, local learner 118A ensures that it is expressed in observable basis 116 established by master learner 114. In fact, although in the present example measured redox data 124A is already collected in the appropriate vector and list form by sensor system 708A, in the event it not, it is the job of local learner 118A to make the necessary reformatting and conversion steps. These steps may be performed by distributed learning algorithm 130 or by other resources of local learner 118A prior to passing vectors 112X″ to distributed learning algorithm 130.

Learning algorithm 130 learns an operator matrix OM that will transform between model feature vector 112M′ and measured feature vector 112X″. More precisely, for each time increment learning algorithm 130 learns the form of operator matrix OM that will transform between model feature vector 112M′ valid at that time increment to measured feature vector 112X″ valid at that time increment. FIG. 8B illustrates operator matrix OM at time tᵢ, hence referred to as OM(tᵢ).

In the present example, operator matrix OM(tᵢ) applied to model feature vector 112M′ valid at time tᵢ, referred to as x(tᵢ), will yield measured feature vector 112X″ valid at time tᵢ, referred to as \hat{x}(t). Of course, the inverse or a related transformation can be encoded in operator matrix OM as long as the changes between the measured and model vectors are captured. Those skilled in the art will recognize that many alternatives exist. Furthermore, the techniques for deriving, calculating or estimating operator matrix OM(tᵢ) can take advantage of techniques beyond typical deep learning processes and including perturbative approaches as well as directed annealing.

To estimate operator matrix OM(tᵢ) for each time increment in the time series it is helpful to work with a high temporal resolution or many time slices. It is thus preferred to provide a high-resolution time series 112XS' in model redox data 112 from reference bioprocess model 106 (see FIG. 7). Thus, model cell line 710 in reference bioreactor 712 should be monitored often throughout its life to produce such a high-resolution time series 112XS′. This period typically includes the lag phase, the growth phase, the stationary phase and the death phase of model cell line 710. The samples throughout these phases can be taken at regular time intervals or even at shorter time intervals during periods of high activity. As mentioned above, the actual measurements of redox indicators and any other features can take advantage of precision measurement instruments such as a high-resolution mass spectrometer, to measure the concentration of a range of different compounds at different masses for each time slice. The results of the measurements can be represented as a heat-map, one image per sample.

Learning algorithm 130 is applied to estimating operator matrix OM in high-resolution temporal steps. The operator matrix OM for each time slice transforms between model feature vectors 112M′ and measured feature vectors 112X″. As a result, each operator matrix OM encodes a redox-related context adjustment during the given time interval. In other words, the context which includes local conditions and any other contingencies and factors that are redox-related is at least partly represented by operator matrix OM.

By obtaining a temporal succession of operator matrices OM learning algorithm 130 effectively encodes step-wise changes in the bioprocess occurring to cell line 702A under local conditions 704A within local bioreactor 706A. As encoded by each operator matrix OM, the succession of changes is taken to represent redox related context adjustments. Preferably, the series of operator matrices OM that encode the progression are stored by system 700 for later use when the same or sufficiently similar local conditions 704A are encountered.

By tracking a succession of changes in redox status and correspondent redox-related context adjustments to cell line 702A under local conditions 704A learning algorithm 130 effectively learns the entire local redox-related bioprocess. As stated, the redox-related context adjustments are encoded in the corresponding operator matrices OM acting of the canonical form of model feature vector 112M′ at each step.

Once learned, operator matrices OM corresponding to specific steps and are stored by learning system 700 for later use. Additional labels may be attached to them for convenience and to simplify any searches that system 700 may need to undertake to find them when required. For example, previously learned operator matrices OM can be provided directly in reference bioprocess model 106 to learning algorithm 130 in master learner 114 to avoid having to re-learn them. Thus, the appropriate operator matrix OM may be included in contingency list 112E*.

It is important to note that a change in redox-related context at any step may lead to an irreversible change. Such change in local biological entity 702A and its locally experienced bioprocess may not be reversible. Those skilled in the art will recognize that one way to express irreversibility is with operator matrices OM that are non-invertible (e.g., projection matrices). Thus, for example, if the change leads to an irreversible process, e.g., apoptosis in all cells in cell line 702A, then this change can be recorded by an operator matrix OM that sends model feature vector 112M′ to zero.

Referring back to FIG. 7, we note that each local bioreactor 706A, 706B, . . . , 706X is equipped with its own actuator system 714A, 714B, . . . , 714X. Each one of actuator systems 714A, 714B, . . . , 714X deploys one or more individual actuators or input mechanisms to control, provide inputs or, in any other way, alter or adjust the bioprocess transpiring in its local cell line 702A, 702B, . . . , 702X housed in corresponding local bioreactor 706A, 706B, . . . , 706X. Individual actuators 714AA and 714AZ of actuator system 714A are specifically designated in FIG. 7 for clarity. In the present example, actuators 714AA and 714AZ are an input or inlet pipe and a stirrer. It is understood that other devices, control mechanism or actuators, especially those affecting redox status are included in actuator system 714A. In prior embodiments, actuators were deployed to adjust the bioprocess.

In the present example, as shown in more detail in the diagram of FIG. 8C, actuator system 714A is also used to adjust the bioprocess as a part of a local feedback mechanism 716A. Local feedback mechanism 716A is provided between local learner 118A and local biological entity 702A in local bioreactor 706A. In the present example, local feedback mechanism 716A includes a connection 718A to local learner 118A and a control unit 720A. Control unit 720A can control individual actuators such as actuators 714AA, 714AZ of actuator system 714A to adjust the conditions in local bioreactor 706A.

In particular, local feedback mechanism 716A can apply the redox related context adjustment discovered by learning algorithm 130 and expressed in the form of operator matrix OM to local biological entity 702A. By context we understand any and all parameters, conditions and circumstances that may affect the redox status of the bioprocess being experienced in bioreactor 706A by local biological entity 702A. The actuators or devices of actuator system 714A may be configured to operate on at least one control parameter that affects local conditions 704A and hence the conditions under which local biological entity 702A experiences the bioprocess. The control parameter or parameters may relate directly to the redox state of the bioprocess.

Actuators or devices 714A-Z of local feedback mechanism 716A are preferably configured to operate more than just one control parameter, condition or circumstance that affects local conditions 704A. The one or more control parameters, conditions and circumstances will typically relate directly to the redox state of the bioprocess experienced by biological entity 702A. Thus, in general, a useful control parameter can be a redox active compound or an electron balance influencer, or still other input that can act upon the bioprocess transpiring in biological entity 702A under local conditions 704A. In general, and independent of the selection of control parameters, and observable redox indicators redox data collected from biological entity 702A should contain at least one known and reliable redox indicator and at least one well known electron balance influencer.

Well established and commonly accepted redox indicators may also be referred to as electron balance indicators. Particularly useful and established electron balance indicators include indicators consisting of an oxidoreductase, an oxidoreductase co-factor, an electron balance influencer compound, an electron balance influencer composition, a redox-active compound, a pK value, a pH value, a threshold value, a context measure and a soft indicator. As already discussed in previous embodiments, local feedback mechanism 716A is capable of acting on any of these redox indicators.

Furthermore, it is known that useful redox indicators or electron balance indicators should be measured or acted upon on short time scales in comparison to GPR times. Hence, in advantageous embodiments, the at least one electron balance indicator is measured or acted upon with a frequency of at least once every hour, at least once every 30 minutes, at least once every 10 minutes, at least once every 5 minutes, at least once every minute, at least once every 30 seconds, at least once every 10 seconds, at least once every 5 seconds, at least once every second, at least twice every second, at least 5 times every second, at least 10 times every second, at least 20 times every second, at least 50 times every second, at least 100 times every second, or more.

In an advantageous embodiment, learning system 700 is set up to capture the bioprocess experienced by local biological entity 702A in a progression of operator matrices OM and associate redox-related context adjustments to be executed by local feedback mechanism 716A with each one of them. More precisely, learning algorithm 130 learns redox-related context adjustments associated with any given operator matrix OM by determining what actions performed by actuators 714A-Z on the one or more control parameters, conditions and circumstances will compensate or reverse the redox state of the bioprocess experienced by biological entity 702A. Still differently put, learning algorithm 130 attempts to learn which control parameters, conditions and circumstances to alter and by how much in order to recover initial conditions of the redox state of the bioprocess at the start of every time increment. It is those redox-related context adjustments that are encoded by the corresponding operator matrices OM acting on the canonical form of model feature vector 112M′. In other words, redox-related context adjustments are taken as being at least partly represented by operator matrices OM.

Once learned, operator matrices OM corresponding to redox-related context adjustments represented by specific alterations in local conditions 704A or are stored by learning system 700 for later use. Preferably, they are stored with the reference bioprocess model 106 for later recall when similar local conditions and local biological entities are being tracked. Additional labels may be attached to operator matrices OM for convenience and to simplify any searches that system 700 may need to undertake to find them when required. For example, previously learned operator matrices OM can be provided directly from portion of learning algorithm 130 residing in reference bioprocess model 106 to learning algorithm 130 in master learner 114 to avoid having to re-learn them. Thus, the appropriate operator matrix OM may be included in contingency list 112E* sent to local learner 118A and any other local learners 118B-X as seen in the present embodiment.

It is important to note that redox-related context adjustments applied by actuators 714A-Z of local feedback mechanism 716A to local conditions 704A may lead to an irreversible change in the bioprocess. Such change in the bioprocess in local biological entity 702A is represented by an operator matrix OM that captures an irreversible step in the bioprocess. Hence, the above approach of attempting to learn the redox-related context adjustments by determining how to reverse the action of this type of operator matrix OM will not work. Those skilled in the art will recognize that one way to express irreversibility is with operator matrices OM that are non-invertible (e.g., projection matrices). Thus, for example, if the step in the bioprocess is irreversible process, e.g., apoptosis in all cells in cell line 702A, then one may conveniently record this change by an operator matrix OM that sends model feature vector 112M′ to zero in its final form 112M′*.

In some cases, any redox-related context adjustments in local conditions 704A under which biological entity 702A undergoes the bioprocess will affect the bioprocess in a way that is not simply reversible. Again, no reversible operator matrix OM may be able to encode for such situations. Persons skilled in the art sometimes refer to this type of process as path dependent. Perturbations that are path dependent are typically expressed with operator matrices OM that are not commutative. Some persons skilled in the art will associate such path dependence with the order of perturbations (order effect) and even specific types of order effects, such as hysteresis.

When the step in the bioprocess is reversible, then the redox-related context adjustments can restore the bioprocess to initial conditions at the start of the time increment. In such cases, mechanism 716A will be able to apply the inverse of the redox-related context adjustment to local conditions 704A and bring the local biological entity 702A back to initial state. More simply put, by making mechanism 716A reverse the redox-related context adjustments the initial redox-related context can be re-established. In the more complicated cases that are irreversible or not simply reversible, the application of the inverse of the redox-related context adjustment may not be possible or may not bring the model conditions back to baseline redox-related context. In any event, persons skilled in the art will be familiar with a host of other types of processes that can be encoded in corresponding operator matrices OM, their inverses, and compositions.

In certain cases, local feedback mechanism 716A will be a secondary feedback loop established between local learner 118A and local biological entity 702A. Of course, local feedback mechanism 716A should be appropriately provisioned to perform any local conditions adjustment represented by operator matrix OM and encoding the redox-related context adjustment.

Returning to FIG. 7, we note that learning system 700, in addition to or instead of using labels, may be equipped with a context classifier for associating operator matrices OM discovered by learning algorithm 130 with local conditions 704A-X given the context matrix CM previously applied to derive model feature vector 112M′. In other words, system 700 may use context classifiers that associate a specific operator matrix OM that transforms from model feature vector 112M′ projected to be appropriate under lab or model conditions to specific local conditions 704A-X in which the given local biological entity 702A-X is embedded. Such context classifiers may further associate any given operator matrix OM with a diagnosis of the corresponding local biological entity 702A-X. For convenience, the context classifier may further associate operator matrices OM with context labels for easier accessing, sharing and searching.

In general, the local biological entity undergoing or experiencing the bioprocess can cover many types of entities. These range from cells, cell lines, cell cultures to biomasses. Any of these may experience the bioprocess in a bioreactor or in another appropriate vessel or in vivo. Local biological entities may also be embodied by living entities, such as plants, organisms, animals, and human subjects. Many of these will typically experience the bioprocess under their standard local conditions, e.g., in their natural habitats.

FIG. 8D illustrates an embodiment of learning system 700 as introduced in FIG. 7 that uses a particularly simple context matrix CM. The relevant portions of system 700 shown for reasons of clarity include just the portion operating under local conditions 704A where biological entity 702A is undergoing the bioprocess it its local bioreactor 706A.

In the present embodiment, rather than relying on context matrix CM being provided from reference bioprocess model 106 as a part of model redox data 112, measured redox data 124A collected by sensor system 708A from local biological entity 702A is used to derive the context. Measured redox data 124A is already collected in the appropriate vector and list form by sensor system 708A. One of the measured redox feature vectors 112X″ is shown explicitly for the measurement collected at the i-th time interval and thus valid at time tᵢ. This vector is referred to as \hat{x}(tᵢ) according to the notation already introduced above.

Once delivered to local learner 118A, vector 112X″ valid at time tᵢ, i.e., \hat{x}(t), is compared with all of its previous forms obtained at other times by learning algorithm 130. From these, learning algorithm 130 verifies that all entries x₁, x₂, . . . , X_{q} of vector \hat{x}(tᵢ) vary in redox-related ways or in the ways that redox indicators in those entries are expected to vary. Furthermore, learning algorithm 130 ensures that all vectors 112X″ being compared are in observable basis 116. After performing these steps and any other typical data conditioning on vectors 112X″, algorithm 130 derives an estimated optimal feature vector 722. Optimal feature vector 722 is a local estimate by learning algorithm 130 of optimal redox data based only on measurements that can actually be performed locally.

To define the corresponding context matrix CM, learning algorithm 130 expands optimal feature vector 722. Specifically, algorithm 130 multiplies the column form of vector 722, referred to as x(t), and the row form of vector 722, referred to as x^{T}(tᵢ) or the transpose, to obtain context matrix CM. Those skilled in the art will recognize that this operation, often called taking the outer product, corresponds to derivation of a projection matrix or projection operator. Thus, context matrix CM is a matrix whose operation on vectors will only pick out their projection into the subspace occupied by vectors 112X″ and also by optimal feature vectors 722.

Locally defined context matrix CM thus picks out only redox-related features and parameters that can be measured by sensor system 708A and drops any other entries. It is useful to label such locally defined context matrix CM for future use by learning system 700. A context label 724 assigned to context matrix CM by local learner 118A can express the most important components of optimal feature vectors 722 associated with context matrix CM. In addition, context label 724 may be associated to all operator matrices OM that are learned within the context defined by context matrix CM.

For example, when only redox data from local cell line 702A is picked out by context matrix CM, then the most important redox indicators from cell line 702A may be used. When the bioprocess being studied involves aging of cell line 702A two appropriate components or entries of optimal feature vector 722 to choose could be GSH and GSSH. The ratio of reduced to oxidized glutathione, namely GSH/GSSH, is well conserved throughout biology and can be thus used in context label 724.

In addition to providing context label 724, it will often be advantageous to associate operator matrices OM discovered or learned by learning algorithm 130 with context classifiers, diagnoses, and other useful annotations. In the example shown in FIG. 8D, a context classifier 726 is attached to context matrix CM. Context classifier 726 includes a list of all operator matrices OM for the full or complete time series (all time intervals or time slices) during which the bioprocess is experienced by local biological entity 702A. Also, a diagnosis can be included either with classifier 726 or separately.

We return now to FIG. 7. Here we see that it is also advantageous to send the context label 724 and context classifier 726 along with any of the additional information back to reference bioprocess model 106. This can be done by transmitting context matrix CM, label 724 and classifier 726 via primary feedback loop 154 to master learner 114, and from there and from there to reference bioprocess model 106. In the present example, this is accomplished by including context matrix CM, label 724 and classifier 726 in the adjustment or update 134 sent to bioprocess reference model 106. This is effectively done when a corresponding reference feedback mechanism 740 is established between master learner 114, reference bioprocess model 106 and reference biological entity 710.

When using redox indicators in label 724 there exists the option of their removal. Specifically, any redox indicator that is stable or unchanging under all redox-related context adjustments encoded by corresponding operator matrices OM in a given context may be removed. The learning required to determine whether a redox indicator can be removed can be performed by any well-known deep learning technique known to those skilled in the art. Preferably, the learning is based on many instances of the bioprocess transpiring in the same local biological entity under varying local conditions and perturbations.

In fact, learning system 700 may implement a single cell line and focus its learning by slightly varying local conditions 704A-X while all local biological entities 702A-X are selected from the same cell line. Under these circumstances, learning system 700 could study different local conditions 704A-X and application of redox-related context adjustments to cell lines 702A-X in an attempt to learn redox-related status of the bioprocess even under conditions not yet known to reference biological model 106. Such learning could be used to improve the reference biological model 106. Furthermore, using the same approach differences between cell lines that are in the same subspace as defined by context matrix CM can be studied and compared with each other.

It should be noted that context matrix CM can be constructed using any number of redox indicators as well as other data entries that may contain environmental parameters with no clear relationship to redox status of the bioprocess. The learning process applied by learning algorithm 130 may be analogous in any of these cases. It is also important to check that during the learning process the vectors being operated on remain in the observable basis 116. When studying cell lines 702A-X as well as any control parameters, factors or conditions that may affect redox status of the bioprocess it is important to maintain observable basis 116 whenever possible. Stability of observable basis 116 is important because it will typically improve later comparisons and further learning.

Learning system 700 can employ many general methods that extend beyond the method used by learning algorithm 130. In other words, learning algorithm 130 that engages in learning the optimal composition of measured redox data, optimal feature vector, observable redox indicators, context matrices CM and operator matrices OM, which may start from a general set of redox indicators and perturbation models, need not be implemented within any one particular learning paradigm. In fact, learning system 700 can employ one or more learning methods. Some particularly useful methods in the embodiments of the present invention include Artificial Intelligence (AI) methods, Hidden Markov methods and Deep Learning (multi-layered neural network) methods. Any of these methods can be implemented in the recursive feedback structure presented by learning systems of the invention.

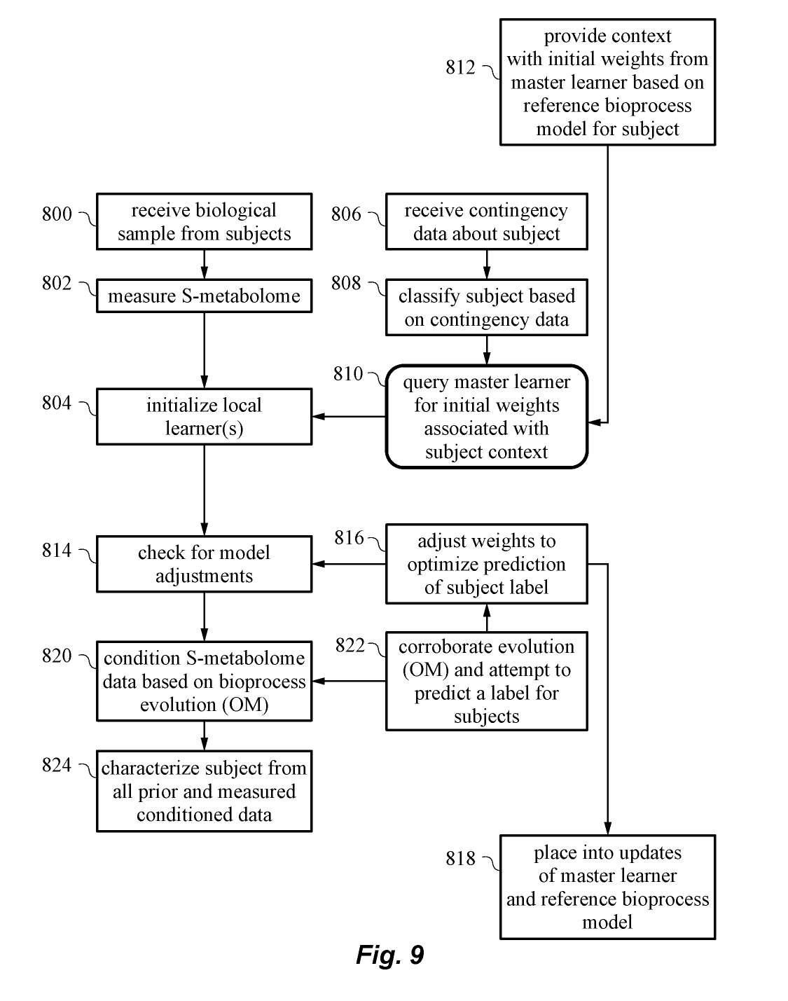

FIG. 9 is a general flow diagram that indicates how learning system 700 of FIG. 7 can be deployed when local biological entities 702A-X are biological samples, e.g., cell lines, from a live subject or multiple live subjects. The subject or subjects can be human.

In a first step 800, subjects provide their biological samples 702A-X to be studied and characterized by a lab. In a second step 802, samples 702A-X are measured by a suitable apparatus available at the lab to obtain their S-metabolome. The S-metabolome will contain the redox indicators that will be used in the present example as entries in optimal feature vector 722.

Once the S-metabolome is known, the samples are placed under local conditions 704A-X. These could be at the same location and in the same facility or at different locations and at different facilities. Furthermore, local conditions 704A-X may be all the same or they may be different.

In step 804 the one or more local learners are initialized with the best estimate of the context matrix CM and optimal feature vector 722. These may be the ones obtained in steps 800 and 802 in studying and analyzing present samples 702A-X.

Alternatively, contingency data including context matrix CM and optimal feature vector 722 for the subject or subjects can be received from reference bioprocess model 106 from a previous test of the same subject(s) or similar subjects studied in the past. For example, contingency data from tests can be contained in curated reference model redox data 108. This data from past tests is obtained from reference bioprocess model 106 in step 806. Furthermore, this contingency data can be used to classify the subject or subjects in step 808 without the need for another measurement of their S-metabolome if the past contingency data is reliable.

As shown in the flow diagram of FIG. 7, step 804 preferably queries master learner 114 for such past contingency data in step 810. Master learner 114, in turn, is in communication with reference bioprocess model 106 for such past contingency data that may be available in step 810.

Alternatively, as shown in step 812, master learner 114 may provide the contingency data to initialize local learners 118A-X in step 804 based on model reference bioprocess 106 for the subject or subjects that is not based on curated reference model redox data 108. This could happen, for example, if a similar subject or subjects is/are presently being tested in the lab under model conditions and is/are providing model redox data 152. Preferably, context matrix CM obtained from reference bioprocess model 106 includes weighting, normalization and other data conditioning functions. These can be applied by a proper composition of corresponding matrices, as is well-known in the art.

The actual tracking of local bioprocesses under local conditions 704A-X commences at step 814. At that step, a check for any model adjustments is conducted. This includes confirmation of proper normalization, weighting, scaling and other data conditioning of context matrix CM and optimal feature vector 722. Any such adjustments, if required, are applied in step 816. These may take place in local learners 118A-X. The adjustments should be helpful in the prediction of subject label 724, if possible. Furthermore, the adjustments should also be communicated back to master learner 114 and also to reference bioprocess model 106 for any updates, as shown in step 818.

Step 820 follows the model adjustments of step 814 are complete. In step 820 S-metabolome data as represented by a time series of measured redox data in vector form according to model redox vector 722 is collected. The temporal progress of the bioprocess in local samples 702A-X is encoded in corresponding series of operator matrices OM.

The results found in step 820 can be corroborated with any previous predictions based on alternative learning approaches used by learning algorithm 130. This corroboration is performed in step 822. The corroboration step preferably also attempts to predict label 724.

The last step 824 involves the characterization of subjects from all prior tests, corroborations, and presently measured redox data. It is noted, that one can undertake an assessment of the efficacies and accuracy of alternative learning methods at step 824.

The above teachings are provided as reference to those skilled in the art in order to explain the salient aspects of the invention. It will be appreciated from the above disclosure that a range of variations on the above-described examples and embodiments may be practiced by the skilled artisan without departing from the scope of the invention(s) herein described. The scope of the invention should therefore be judged by the appended claims and their equivalents.

1. A learning system for learning a redox-related context adjustment to a bioprocess having hidden states, said learning system comprising:
a) a reference bioprocess model configured to yield model redox data for said bioprocess;
b) a master learner configured to receive said model redox data and to establish therefrom:
i) an observable basis of redox indicators; and
ii) a model feature vector comprising said model redox data expressed in said observable basis;
c) at least one local biological entity undergoing said bioprocess under local conditions and generating measured redox data for said bioprocess;
d) a local learner configured to:
i) receive said measured redox data and at least a portion of said model redox data; and
ii) express said measured redox data by a measured feature vector in said observable basis;
wherein said learning system deploys a learning algorithm to learn an operator matrix for transforming between said model feature vector and said measured feature vector, said redox-related context adjustment comprising said operator matrix.

2. The learning system of claim 1, wherein said reference bioprocess model is obtained from a reference biological entity undergoing said bioprocess under model conditions.

3. The learning system of claim 1, wherein said at least one local biological entity undergoing said bioprocess comprises a live subject.

4. The learning system of claim 1, wherein said at least one local biological entity undergoing said bioprocess is in a reference bioreactor.

5. The learning system of claim 1, further comprising a context classifier for associating said operator matrix with said local conditions.

6. The learning system of claim 5, wherein said context classifier further associates said operator matrix with a diagnosis of said local biological entity.

7. The learning system of claim 5, wherein said context classifier further associates said operator matrix with a context label.

8. The learning system of claim 1, further comprising a local feedback mechanism between said local learner and said at least one local biological entity for applying said redox-related context adjustment to said local biological entity.

9. The learning system of claim 8, wherein said local feedback mechanism comprises at least one actuator configured to operate on at least one control parameter, said at least one control parameter being selected from the group consisting of redox active compounds and electron balance influencers.

10. The learning system of claim 9, wherein said electron balance indicator is selected from a group of indicators consisting of an oxidoreductase, an oxidoreductase co-factor, an electron balance influencer compound, an electron balance influencer composition, a redox-active compound, a pK value, a pH value, a threshold value, a context measure and a soft indicator.

11. The learning system of claim 9, wherein said electron balance indicator is measured at least once every 5 minutes, at least once every minute, at least once every 30 seconds, at least once every 10 seconds, at least once every 5 seconds, at least once every second, at least twice every second, at least 5 times every second, at least 10 times every second, at least 20 times every second, at least 50 times every second, at least times every second, or more.

12. The learning system of claim 8, wherein said local feedback mechanism is in a secondary feedback loop between said local learner and said at least one local biological entity.

13. The learning system of claim 8, wherein said local feedback mechanism performs a local conditions adjustment based on said operator matrix.

14. The learning system of claim 1, wherein said learning system employs at least one learning method selected from the group consisting of an Artificial Intelligence method, a hidden Markov method, a Deep Learning method.

15. The learning system of claim 1, wherein said model redox data and said measured redox data comprises at least one electron balance influencer.

16. A method for learning a redox-related context adjustment to a bioprocess having hidden states, said method comprising:
a) obtaining model redox data for said bioprocess from a reference bioprocess model;
b) transmitting said model redox data to a master learner configured to establish therefrom:
i) an observable basis of redox indicators;
ii) a model feature vector comprising said model redox data expressed in said observable basis;
c) placing at least one local biological entity under local conditions for undergoing said bioprocess and for generating measured redox data for said bioprocess;
d) configuring a local learner to:
i) receive said measured redox data and at least a portion of said model redox data; and
ii) express said measured redox data by a measured feature vector in said observable basis;
e) deploying a learning algorithm to learn an operator matrix for transforming between said model feature vector and said measured feature vector, said redox-related context adjustment comprising said operator matrix.

17. The method of claim 16, further comprising the step of associating said operator matrix with said local conditions by a context classifier.

18. The method of claim 17, wherein said context classifier further associates said operator matrix with a diagnosis of said local biological entity.

19. The method of claim 17, wherein said context classifier further associates said operator matrix with a context label.

20. The method of claim 16, further comprising the step of applying said redox-related context adjustment to said local biological entity by a local feedback mechanism.

21. The method of claim 20, wherein said step of applying said redox-related context adjustment comprises operating on at least one control parameter, said at least one control parameter being selected from the group consisting of redox active compounds and electron balance influencers.

22. The method of claim 21, wherein said electron balance indicator is selected from a group of indicators consisting of an oxidoreductase, an oxidoreductase co-factor, an electron balance influencer compound, an electron balance influencer composition, a redox-active compound, a pK value, a pH value, a threshold value, a context measure and a soft indicator.

23. The method of claim 21, wherein said electron balance indicator is measured at least once every minutes, at least once every minute, at least once every 30 seconds, at least once every 10 seconds, at least once every 5 seconds, at least once every second, at least twice every second, at least 5 times every second, at least 10 times every second, at least 20 times every second, at least 50 times every second, at least 100 times every second, or more.

24. The method of claim 16, wherein said learning employs at least one learning method selected from the group consisting of an Artificial Intelligence method, a hidden Markov method, a Deep Learning method.
